(19) United States
(12) Patent Application Publication
(10) Pub. No.: US 2024/0035055 A1
(43) Pub. Date: Feb. 1, 2024

(54) PROCESS FOR PURIFICATION OF AN OLIGOSACCHARIDE SOLUTION PRODUCED BY CELL CULTIVATION OR MICROBIAL FERMENTATION

(71) Applicant: INBIOSE N.V., Zwijnaarde (BE)

(72) Inventors: Joeri Beauprez, Zwijnaarde (BE); Maarten Delforche, Zwijnaarde (BE); Gaspard Lequeux, Zwijnaarde (BE); Wim Soetaert, Zwijnaarde (BE); Thomas Verbrugghen, Zwijnaarde (BE)

(21) Appl. No.: 18/041,054

(22) PCT Filed: Aug. 10, 2021
(86) PCT No.: PCT/EP2021/072273

§ 371 Date: Feb. 8, 2023

(30) Foreign Application Priority Data
Aug. 10, 2020 (EP) 20190210.3
Aug. 10, 2021 (EP) 20190210.3

(51) Int. Cl. C12P 19/04; B01D 15/36; C07H 1/08; B01D 61/42; B01D 61/14; B01D 61/02

(52) U.S. Cl. CPC C12P 19/04; B01D 15/362; C07H 1/08; B01D 61/422; B01D 61/146; B01D 61/027

(57) ABSTRACT

This disclosure is in the technical field of cell cultivation or fermentation for the production of oligosaccharides. The present application discloses a process for purification of an oligosaccharide solution produced by microbial fermentation.

### CROSS-REFERENCE TO RELATED APPLICATIONS

This application is a national phase entry under 35 U.S.C. § 371 of International Patent Application PCT/EP2021/072273, filed Aug. 10, 2021, designating the United States of America and published as International Patent Publication WO 2022/034079 A1 on Feb. 17, 2022, which claims the benefit under Article 8 of the Patent Cooperation Treaty to European Patent Application Serial No. 20190210.3, filed Aug. 10, 2020.

### TECHNICAL FIELD

This disclosure is in the technical field of cell cultivation or fermentation for the production of oligosaccharides. The present application discloses a process for purification of an oligosaccharide solution produced by a cell cultivation or microbial fermentation.

### BACKGROUND

Oligosaccharides, often present as glyco-conjugated forms to proteins and lipids, are involved in many vital phenomena such as differentiation, development and biological recognition processes related to the development and progress of fertilization, embryogenesis, inflammation, metastasis, and host pathogen adhesion. Oligosaccharides can also be present as unconjugated glycans in body fluids and human milk wherein they also modulate important developmental and immunological processes (Bode, Early Hum. Dev. 1-4 (2015); Reily et al., Nat. Rev. Nephrol. 15, 346-366 (2019); Varki, Glycobiology 27, 3-49 (2017)). For example, several oligosaccharides have proven to act as decoys to reduce the risk of infections by bacterial and viral pathogens adhering to mammal cells by binding to these cells' surface glycoproteins. Nowadays, oligosaccharides are produced on an industrial scale either chemically, by chemo-enzymatic synthesis or by cultivation or fermentation of (metabolically engineered) cells or micro-organisms. After production, the oligosaccharide preferably is purified to be added in the respective application.

To take advantage of the positive effects of specific oligosaccharides, individual oligosaccharides are being added to nutritional compositions, cosmetics, pharmaceutical compositions and plant protection products. In some instances, supplementing with a combination of different oligosaccharides is more convenient, as such compositions e.g., more closely resemble the natural source of the oligosaccharides in case the oligosaccharide mixture is a mixture of mammalian milk oligosaccharides. In other cases a mix of specific oligosaccharides is produced more efficiently in a simpler manner by producing the mixture of oligosaccharides in one fermentation and purifying the mixture of oligosaccharides all together from the biomass, medium components and contaminants, without separating the different oligosaccharides from each other.

### SUMMARY OF THE DISCLOSURE

Provided is a purified oligosaccharide solution, whether comprising only one oligosaccharide to be purified or a mixture of different oligosaccharides to be purified, wherein the process is applicable in industrial scale production and provides a simplified purification process.

Provided is a process for purification of an oligosaccharide solution produced by a cell cultivation or microbial fermentation.

In a first aspect, a process for purification of an oligosaccharide solution produced by a cell cultivation or microbial fermentation is provided.

In a second aspect, a process purification of an oligosaccharide solution produced by a cell cultivation or microbial fermentation is provided, wherein the oligosaccharide solution has an ash content equal to or lower than 10.

In a third aspect, a process to provide a syrup of an oligosaccharide solution is provided with a Brix between 8 and 75% is provided.

In a fourth aspect, a dried powder essentially comprising or containing one oligosaccharide or a mixture of structurally distinct oligosaccharides is provided, preferably for the production of a nutritional composition, a dietary supplement, a pharmaceutical ingredient, and/or a cosmetics ingredient.

In a fifth aspect, a nutritional composition comprising a dried powder that essentially comprises or contains one oligosaccharide or a mixture of structurally distinct oligosaccharides is provided.

### Definitions

The words used in this specification to describe this disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of this disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, purification steps are performed according to the manufacturer's specifications.

In the specification, there have been disclosed embodiments of this disclosure, and although specific terms are employed, the terms are used in a descriptive sense only and not for purposes of limitation, the scope of this disclosure being set forth in the following claims. It must be understood that the illustrated embodiments have been set forth only for the purposes of example and that it should not be taken as limiting this disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

In this document and in its claims, the verb “to comprise” and its conjugations is used in its non-limiting sense to mean that items following the word are included, but items not specifically mentioned are not excluded. Throughout the application, the verb “to comprise” may be replaced by “to consist” or “to consist essentially of” and vice versa. In addition the verb “to consist” may be replaced by “to consist essentially of” meaning that a composition as defined herein may comprise additional component(s) than the ones specifically identified, the additional component(s) not altering the unique characteristic of this disclosure. In addition, reference to an element by the indefinite article “a” or “an” does not exclude the possibility that more than one of the element is present, unless the context clearly requires that there be one and only one of the elements. The indefinite article “a” or “an” thus usually means “at least one.” Throughout the application, unless explicitly stated otherwise, the articles “a” and “an” are preferably replaced by “at least two,” more preferably by “at least three,” even more preferably by “at least four,” even more preferably by “at least live,” even more preferably by “at least six,” most preferably by “at least seven.”

Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference. The full content of the priority application EP20190210 is also incorporated by reference to the same extent as if the priority application was specifically and individually indicated to be incorporated by reference.

The terms “recombinant” or “transgenic” or “metabolically engineered” or “genetically modified,” as used herein with reference to a cell or host cell are used interchangeably and indicates that the cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene, or are described to be transformed by the introduction of at least one heterologous or exogenous gene. Metabolically engineered or recombinant or transgenic cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one that has been produced by a recombinant cell. A “heterologous sequence” or a “heterologous nucleic acid,” as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host microorganism cell, wherein techniques may be applied that will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” cell or microorganism as used within the context of this disclosure refers to a cell or microorganism that is genetically modified.

The term “endogenous,” within the context of this disclosure refers to any polynucleotide, polypeptide or protein sequence that is a natural part of a cell and is occurring at its natural location in the cell chromosome and of which the control of expression has not been altered compared to the natural control mechanism acting on its expression. The term “exogenous” refers to any polynucleotide, polypeptide or protein sequence that originates from outside the cell under study and not a natural part of the cell or that is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “wild type” refers to the commonly known genetic or phenotypical situation as it occurs in nature.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, Sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, glucose, galactose, N-acetylglucosamine, glucosamine, mannose, xylose, N-acetylmannosamine, N-acetylneuraminic acid, N-glycolylneuraminic acid, a sialic acid, N-acetylgalactosamine, galactosamine, fucose, rhamnose, glucuronic acid, gluconic acid, fructose and polyols.

With the term polyol is meant an alcohol containing multiple hydroxyl groups. For example, glycerol, sorbitol, or mannitol.

The term “disaccharide” as used herein refers to a saccharide composed of two monosaccharide units. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal and fucopyranosyl-(1-4)-N-glycolylneuraminic acid (Fuc-(1-4)-Neu5Gc).

“Oligosaccharides” are glycan structures that are composed of three or more monosaccharide subunits that are linked to each other via glycosidic bonds in a linear or in a branched structure. “Oligosaccharide” as the term is used herein refers to a saccharide polymer containing a small number, typically three to twenty, of simple sugars, i.e., monosaccharides. Preferably, the oligosaccharide as described herein contains monosaccharides selected from the list as used herein above. Examples of oligosaccharides include but are not limited to Lewis-type antigen oligosaccharides, sialylated oligosaccharides, fucosylated oligosaccharides, chitosan, chitosan oligosaccharide, sulphated chitosan, acetylated chitosan, heparosan, chondroitin sulphate, glycosaminoglycan oligosaccharide, heparin, heparan sulphate, chondroitin sulphate, dermatan sulphate, hyaluronan or hyaluronic acid, keratan sulphate mammalian milk oligosaccharides and human milk oligosaccharides.

As used herein, “mammalian milk oligosaccharide” (MMO) refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-neotetraose d, sialyllacto-N-neotetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-neotetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides. Mammalian milk oligosaccharides (MMOs) comprise oligosaccharides present in milk found in any phase during lactation including colostrum milk from humans (i.e., human milk oligosaccharides or HMOs) and mammals including but not limited to cows (Bos Taurus), sheep (Ovis aries), goats (Capra aegagrus hircus), bactrian camels (Camelus bactrianus), horses (Equus ferns caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus Vulpecula), koalas (Phascolarctos cinereus), eastern quolls (Dasyurus viverrinus), platypus (Ornithorhynchus anatinus). Human milk oligosaccharides (HMOs) are also known as human identical milk oligosaccharides that are chemically identical to the human milk oligosaccharides found in human breast milk but that are biotechnologically-produced (e.g., using cell free systems or cells and organisms comprising a bacterium, a fungus, a yeast, a plant, animal, or protozoan cell, preferably genetically engineered cells and organisms). Human identical milk oligosaccharides are marketed under the name HiMO.

As used herein, “lactose-based mammalian milk oligosaccharide (MMO)” refers to a MMO as defined herein that contains a lactose at its reducing end.

As used herein the term “Lewis-type antigens” comprise the following oligosaccharides: H1 antigen, which is Fucα1-2Galβ1-3GlcNAc, or in short 2′FLNB; Lewisa, which is the trisaccharide Galβ1-3[Fucα1-4]GlcNAc, or in short 4-FLNB; Lewisb, which is the tetrasaccharide Fucα1-2Galβ1-3[Fucα1-4]GlcNAc, or in short DiF-LNB; sialyl Lewisa, which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewisx, which is the trisaccharide Galβ1-4[Fucα1-3]GlcNAc, or otherwise known as 3-Fucosyl-N-acetyl-lactosamine, in short 3-FLacNAc, Lewisy, which is the tetrasaccharide Fucα1-2Galβ1-4[Fucα1-3]GlcNAc and sialyl Lewisx, which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

As used herein, a ‘sialylated oligosaccharide’ is to be understood as a charged sialic acid containing oligosaccharide, i.e., an oligosaccharide having a sialic acid residue. It has an acidic nature. A sialylated oligosaccharide contains at least one sialic acid monosaccharide subunit, like e.g., but not limited to Neu5Ac, and Neu5Gc. The sialylated oligosaccharide is a saccharide structure comprising at least three monosaccharide subunits linked to each other via glycosidic bonds, wherein at least one of the monosaccharide subunit is a sialic acid. The sialylated oligosaccharide can contain more than one sialic acid residue, e.g., two, three or more. The sialic acid can be linked to other monosaccharide subunits comprising galactose, GlcNAc, sialic acid, via alpha-glycosidic bonds comprising alpha-2,3, alpha-2,6 linkages. Some examples are 3-SL (3′-sialyllactose), 3′-sialyllactosamine, 6-SL (6′-sialyllactose), 6′-sialyllactosamine, oligosaccharides comprising 6′-sialyllactose, 8,3-disialyllactose (Neu5Ac-a2,8-Neu5Ac-a2,3-Gal-b1,4-Glc), SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, sialyllacto-N-neotetraose c, sialyllacto-N-neotetraose d, 3′-sialyl-3-fucosyllactose, di sialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3 Galβ-1,3 GalNAcβ-1,4(Neu5Acα-2,3)Galβ-1,4Glc, GT1a Neu5Acα-2,8Neu5Acα-2,3 Gal 0-1,3 GalNAcβ-1,4(Neu5Acα-2,3)Galβ-1,4Glc, GD2 GalNAc 0-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3 GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3 Galβ-1,3 GalNAcβ-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAcβ-1,4(Neu5Acα-2,8Neu5Acα-2,8Neu5Acα2,3)Galβ-1,4Glc, GP1c Neu5Acα-2,8Neu5Acα-2,3Galβ-1,3GalNAcβ-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fucα-1,2Galβ-1,3GalNAcβ-1,4(Neu5Acα-2,3)Galβ-1,4Glc; all of which may be extended to the production of the corresponding gangliosides by reacting the above oligosaccharide moieties with ceramide or synthetizing the above oligosaccharides on a ceramide.

The terms “alpha-2,3-sialyltransferase,” “alpha 2,3 sialyltransferase,” “3-sialyltransferase, “α-2,3-sialyltransferase,” “α 2,3 sialyltransferase,” “3 sialyltransferase, “3-ST” or “3 ST” as used in this disclosure, are used interchangeably and refer to a glycosyltransferase that catalyzes the transfer of sialic acid from the donor CMP-Neu5Ac, to the acceptor molecule in an alpha-2,3-linkage. The terms “3′ sialyllactose,” “3′-sialyllactose,” “alpha-2,3-sialyllactose,” “alpha 2,3 sialyllactose,” “α-2,3-sialyllactose,” “α 2,3 sialyllactose,” 3SL” or “3′ SL” as used in this disclosure, are used interchangeably and refer to the product obtained by the catalysis of the alpha-2,3-fucosyltransferase transferring the sialic acid group from CMP-Neu5Ac to lactose in an alpha-2,3-linkage. The terms “alpha-2,6-sialyltransferase,” “alpha 2,6 sialyltransferase,” “6-sialyltransferase, “α-2,6-sialyltransferase,” “α 2,6 sialyltransferase,” “6 sialyltransferase, “6-ST” or “6ST” as used in this disclosure, are used interchangeably and refer to a glycosyltransferase that catalyzes the transfer of sialic acid from the donor CMP-Neu5Ac, to the acceptor molecule in an alpha-2,6-linkage. The terms “6′ sialyllactose,” “6′-sialyllactose,” “alpha-2,6-sialyllactose,” “alpha 2,6 sialyllactose,” “α-2,6-sialyllactose,” “α 2,6 sialyllactose,” 6SL″ or “6′ SL” as used in this disclosure, are used interchangeably and refer to the product obtained by the catalysis of the alpha-2,6-fucosyltransferase transferring the sialic acid group from CMP-Neu5Ac to lactose in an alpha-2,6-linkage. The terms “alpha-2,8-sialyltransferase,” “alpha 2,8 sialyltransferase,” “8-sialyltransferase, “α-2,8-sialyltransferase,” “α 2,8 sialyltransferase,” “8 sialyltransferase, “8-ST” or “8ST” as used in this disclosure, are used interchangeably and refer to a glycosyltransferase that catalyzes the transfer of sialic acid from the donor CMP-Neu5Ac, to the acceptor in an alpha-2,8-linkage.

A ‘fucosylated oligosaccharide’ as used herein and as generally understood in the state of the art is an oligosaccharide that is carrying a fucose-residue. Such fucosylated oligosaccharide is a saccharide structure comprising at least three monosaccharide subunits linked to each other via glycosidic bonds, wherein at least one of the monosaccharide subunit is a fucose. A fucosylated oligosaccharide can contain more than one fucose residue, e.g., two, three or more. A fucosylated oligosaccharide can be a neutral oligosaccharide or a charged oligosaccharide e.g., also comprising sialic acid structures. Fucose can be linked to other monosaccharide subunits comprising glucose, galactose, GlcNAc via alpha-glycosidic bonds comprising alpha-1,2 alpha-1,3, alpha-1,4, alpha-1,6 linkages.

Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNFP I), Lacto-N-fucopentaose II (LNFP II), Lacto-N-fucopentaose III (LNFP III), lacto-N-fucopentaose V (LNFP V), lacto-N-fucopentaose VI (LNFP VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose, 3′-sialyl-3-fucosyllactose, di sialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose.

The terms “alpha-1,2-fucosyltransferase,” “alpha 1,2 fucosyltransferase,” “2-fucosyltransferase, “α-1,2-fucosyltransferase,” “α 1,2 fucosyltransferase,” “2 fucosyltransferase, “2-FT” or “2FT” as used in this disclosure, are used interchangeably and refer to a glycosyltransferase that catalyzes the transfer of fucose from the donor GDP-L-fucose, to the acceptor molecule in an alpha-1,2-linkage. The terms “2′ fucosyllactose,” “2′-fucosyllactose,” “alpha-1,2-fucosyllactose,” “alpha 1,2 fucosyllactose,” “α-1,2-fucosyllactose,” “α 1,2 fucosyllactose,” “Galβ-4(Fucα1-2)Glc,” 2FL” or “2′FL” as used in this disclosure, are used interchangeably and refer to the product obtained by the catalysis of the alpha-1,2-fucosyltransferase transferring the fucose residue from GDP-L-fucose to lactose in an alpha-1,2-linkage. The terms “difucosyllactose,” “di-fucosyllactose,” “lactodifucotetraose,” “2′,3-difucosyllactose,” “2′,3 difucosyllactose,” “α-2′,3-fucosyllactose,” “α 2′,3 fucosyllactose, “Fucα1-2Galβ 1-4(Fucα1-3)Glc,” “DFLac,” 2′,3 diFL,” “DFL,” “DiFL” or “diFL” as used in this disclosure, are used interchangeably.

The terms “alpha-1,3-fucosyltransferase,” “alpha 1,3 fucosyltransferase,” “3-fucosyltransferase, “α-1,3-fucosyltransferase,” “α 1,3 fucosyltransferase,” “3 fucosyltransferase, “3-FT” or “3FT” as used in this disclosure, are used interchangeably and refer to a glycosyltransferase that catalyzes the transfer of fucose from the donor GDP-L-fucose, to the acceptor molecule in an alpha-1,3-linkage. The terms “3-fucosyllactose,” “alpha-1,3-fucosyllactose,” “alpha 1,3 fucosyllactose,” “α-1,3-fucosyllactose,” “α 1,3 fucosyllactose,” “Galβ-4(Fucα1-3)Glc,” 3FL” or “3-FL” as used in this disclosure, are used interchangeably and refer to the product obtained by the catalysis of the alpha-1,3-fucosyltransferase transferring the fucose residue from GDP-L-fucose to lactose in an alpha-1,3-linkage.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′, 3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

The terms “LNB” and “Lacto-N-biose” are used interchangeably and refer to the disaccharide Gal-b1,3-GlcNAc.

The terms “LacNAc” and “N-acetyllactosamine” are used interchangeably and refer to the disaccharide Gal-b1,4-GlcNAc.

The terms “LNT II,” “LNT-II,” “LN3,” “lacto-N-triose II,” “lacto-N-triose II,” “lacto-N-triose,” “lacto-N-triose” or “GlcNAcβ1-3Galβ1-4Glc” as used in this disclosure, are used interchangeably.

The terms “LNT,” “lacto-N-tetraose,” “lacto-N-tetraose” or “Galβ1-3GlcNAcβ1-3Galβ1-4Glc” as used in this disclosure, are used interchangeably.

The terms “LNnT,” “lacto-N-neotetraose,” “lacto-N-neotetraose,” “neo-LNT” or “Galβ1-4GlcNAcβ1-3Galβ1-4Glc” as used in this disclosure, are used interchangeably.

The terms “LSTa,” “LS-Tetrasaccharide a,” “Sialyl-lacto-N-tetraose a,” “sialyllacto-N-tetraose a” or “Neu5Ac-a2,3-Gal-b1,3-GlcNAc-b1,3-Gal-b1,4-Glc” as used in this disclosure, are used interchangeably.

The terms “LSTb,” “LS-Tetrasaccharide b,” “Sialyl-lacto-N-tetraose b,” “sialyllacto-N-tetraose b” or “Gal-b1,3-(Neu5Ac-a2,6)-GlcNAc-b1,3-Gal-b1,4-Glc” as used in this disclosure, are used interchangeably.

The terms “LSTc,” “LS-Tetrasaccharide c,” “Sialyl-lacto-N-tetraose c,” “sialyllacto-N-tetraose c,” “sialyllacto-N-neotetraose c” or “Neu5Ac-a2,6-Gal-b1,4-GlcNAc-b1,3-Gal-b1,4-Glc” as used in this disclosure, are used interchangeably.

The terms “LSTd,” “LS-Tetrasaccharide d,” “Sialyl-lacto-N-tetraose d,” “sialyllacto-N-tetraose d,” “sialyllacto-N-neotetraose d” or “Neu5Ac-a2,3-Gal-b1,4-GlcNAc-b1,3-Gal-b1,4-Glc” as used in this disclosure, are used interchangeably.

The terms “DSLNnT” and “Disialyllacto-N-neotetraose” are used interchangeably and refer to Neu5Ac-a2,6-[Neu5Ac-a2,6-Gal-b1,4-GlcNAc-b1,3]-Gal-b1,4-Glc.

The terms “DSLNT” and “Disialyllacto-N-tetraose” are used interchangeably and refer to Neu5Ac-a2,6-[Neu5Ac-a2,3-Gal-b1,3-GlcNAc-b1,3]-Gal-b1,4-Glc. The terms “LNFP-I,” “lacto-N-fucopentaose I,” “LNFP I,” “LNF I OH type I determinant,” “LNF I,” “LNF1,” “LNF 1” and “Blood group H antigen pentaose type 1” are used interchangeably and refer to Fuc-a1,2-Gal-b1,3-GlcNAc-b1,3-Gal-b1,4-Glc.

The terms “GalNAc-LNFP-I” and “blood group A antigen hexaose type I” are used interchangeably and refer to GalNAc-a1,3-(Fuc-a1,2)-Gal-b1,3-GlcNAc-b1,3-Gal-b1,4-Glc.

The terms “LNFP-II” and “lacto-N-fucopentaose II” are used interchangeably and refer to Gal-b1,3-(Fuc-a1,4)-GlcNAc-b1,3-Gal-b1,4-Glc.

The terms “LNFP-III” and “lacto-N-fucopentaose III” are used interchangeably and refer to Gal-b1,4-(Fuc-a1,3)-GlcNAc-b1,3-Gal-b1,4-Glc.

The terms “LNFP-V” and “lacto-N-fucopentaose V” are used interchangeably and refer to Gal-b1,3-GlcNAc-b1,3-Gal-b1,4-(Fuc-a1,3)-Glc.

The terms “LNFP-VI,” “LNnFP V” and “lacto-N-neofucopentaose V” are used interchangeably and refer to Gal-b1,4-GlcNAc-b1,3-Gal-b1,4-(Fuc-a1,3)-Glc.

The terms “LNnFP I” and “Lacto-N-neofucopentaose I” are used interchangeably and refer to Fuc-a1,2-Gal-b1,4-GlcNAc-b1,3-Gal-b1,4-Glc.

The terms “LNDFH I,” “Lacto-N-difucohexaose I,” “LNDFH-I,” “LDFH I,” “Leb-lactose” and “Lewis-b hexasaccharide” are used interchangeably and refer to Fuc-a1,2-Gal-b1,3-[Fuc-a1,4]-GlcNAc-b1,3-Gal-b1,4-Glc.

The terms “LNDFH II,” “Lacto-N-difucohexaose II,” “Lewis a-Lewis x” and “LDFH II” are used interchangeably and refer to Fuc-a1,4-(Gal-b1,3)-GlcNAc-b1,3-Gal-b1,4-(Fuc-a 1,3)-Glc.

The terms “LNnDFH,” “Lacto-N-neoDiFucohexaose” and “Lewis x hexaose” are used interchangeably and refer to Gal-b1,4-(Fuc-a1,3)-GlcNAc-b1,3-Gal-b1,4-(Fuc-a1,3)-Glc.

The terms “alpha-tetrasaccharide” and “A-tetrasaccharide” are used interchangeably and refer to GalNAc-a1,3-(Fuc-a1,2)-Gal-b1,4-Glc.

“Charged oligosaccharides” are oligosaccharide structures that contain one or more negatively charged monosaccharide subunits including N-acetylneuraminic acid (Neu5Ac), commonly known as sialic acid, N-glycolylneuraminic acid (Neu5Gc), glucuronate and galacturonate. Charged oligosaccharides are also referred to as acid oligosaccharides or acidic oligosaccharides. Sialic acid belongs to the family of derivatives of neuraminic acid (5-amino-3,5-dideoxy-D-glycero-D-galacto-non-2-ulosonic acid). Neu5Gc is a derivative of sialic acid, which is formed by hydroxylation of the N-acetyl group at C5 of Neu5Ac. In contrast, neutral oligosaccharides are non-sialylated oligosaccharides, and thus do not contain an acidic monosaccharide subunit. Neutral oligosaccharides comprise non-charged fucosylated oligosaccharides that contain one or more fucose subunits in their glycan structure as well as non-charged non-fucosylated oligosaccharides that lack any fucose subunit. Other examples of charged oligosaccharides are sulphated chitosans and deacetylated chitosans.

As used herein, an antigen of the human ABO blood group system is an oligosaccharide. Such antigens of the human ABO blood group system are not restricted to human structures. The structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

A ‘fucosylation pathway’ as used herein is a biochemical pathway comprising the enzymes and their respective genes, mannose-6-phosphate isomerase, phosphomannomutase, mannose-1-phosphate guanylyltransferase, GDP-mannose 4,6-dehydratase, GDP-L-fucose synthase and/or the salvage pathway L-fucokinase/GDP-fucose pyrophosphorylase, combined with a fucosyltransferase leading to α 1,2; α 1,3 α 1,4 and/or α 1,6 fucosylated oligosaccharides.

A ‘sialylation pathway’ is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, Glucosamine 6-phosphate N-acetyltransferase, N-AcetylGlucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate-9-phosphate phosphatase, and/or CMP-sialic acid synthase, combined with a sialyltransferase leading to α 2,3; α 2,6 and/or α 2,8 sialylated oligosaccharides.

A ‘galactosylation pathway’ as used herein is a biochemical pathway comprising the enzymes and their respective genes, galactose-1-epimerase, galactokinase, glucokinase, galactose-1-phosphate uridylyltransferase, UDP-glucose 4-epimerase, glucose-1-phosphate uridylyltransferase, and/or glucophosphomutase, combined with a galactosyltransferase leading to an alpha or beta bound galactose on the 2, 3, 4, and/or 6 hydroxyl group of an oligosaccharide.

An ‘N-acetylglucosamine carbohydrate pathway’ as used herein is a biochemical pathway comprising the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridylyltransferase, glucosamine-1-phosphate acetyltransferase, and/or glucosamine-1-phosphate acetyltransferase, combined with a glycosyltransferase leading to an alpha or beta bound N-acetylglucosamine on the 3, 4, and/or 6 hydroxylgroup of an oligosaccharide.

An ‘N-acetylgalactosaminylation pathway’ as used herein is a biochemical pathway comprising at least one of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and/or UDP-N-acetylgalactosamine pyrophosphorylase combined with a glycosyltransferase leading to a GalNAc-modified compound comprising a mono-, di- or oligosaccharide having an alpha or beta bound N-acetylgalactosamine on the mono-, di- or oligosaccharide.

A ‘mannosylation pathway’ as used herein is a biochemical pathway comprising at least one of the enzymes and their respective genes chosen from the list comprising mannose-6-phosphate isomerase, phosphomannomutase and/or mannose-1-phosphate guanylyltransferase combined with a glycosyltransferase leading to a mannosylated compound comprising a mono-, di- or oligosaccharide having an alpha or beta bound mannose on the mono-, di- or oligosaccharide.

An ‘N-acetylmannosaminylation pathway’ as used herein is a biochemical pathway comprising at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to a ManNAc-modified compound comprising a mono-, di- or oligosaccharide having an alpha or beta bound N-acetylmannosamine on the mono-, di- or oligosaccharide.

The term “consisting essentially of” as used herein and as used in the art, refers to compositions comprising the compound(s) specified after the term, and—optionally—inevitable by-products. The inevitable by-products include—for example—compounds that were generated during a cell cultivation or microbial fermentation for the production of the oligosaccharide solution comprising only one or a mixture of oligosaccharides, as well as compounds that were introduced into a process stream from which the oligosaccharide solution is recovered, but that could not have been removed therefrom.

The term “consisting essentially of” with respect to spray-dried powders includes spray-dried powders containing with respect to the dry matter of the spray-dried powder at least 80%-wt., at least 85%-wt., at least 90%-wt., at least 93%-wt., at least 95%-wt. or at least 98%-wt. the oligosaccharide mixture. The term “consisting essentially of” is used likewise with respect to spray-dried powders, process streams and solutions containing the oligosaccharide mixture.

Further herein, the terms “contaminants” and “impurities” preferably mean particulates, cells, cell components, metabolites, cell debris, proteins, peptides, amino acids, nucleic acids, glycolipids and endotoxins that can be present in an aqueous medium from a fermentation process.

The term “clarifying” as used herein refers to the act of treating an aqueous medium or fermentation broth to remove suspended particulates and contaminants from the fermentation process, particularly cells, cell components, insoluble metabolites and debris, that could interfere with the eventual purification of the oligosaccharide solution, oligosaccharide or oligosaccharide mixture. Such treatment can be carried out in a conventional manner by centrifugation, flocculation, flocculation with optional ultrasonic treatment, gravity filtration, microfiltration, foam separation or vacuum filtration (e.g., through a ceramic filter that can include a Celite™ filter aid).

The terms “protein-free oligosaccharide solution” as used herein means an oligosaccharide solution from an aqueous medium or broth from a fermentation process, which medium has been treated to remove substantially all the proteins, as well as any related impurities, such as amino acids, peptides, endotoxins, glycolipids, RNA and DNA, from the process that could interfere with the eventual purification of the oligosaccharide solution from the process. Such removal of proteins, preferably substantially all proteins, can be accomplished in a conventional manner by ion exchange chromatography, affinity chromatography, ultrafiltration, and size exclusion chromatography. Preferably, a protein-free oligosaccharide solution is a clarified oligosaccharide solution or clarified fermentation broth.

The terms “purification of an oligosaccharide solution from the cultivation or fermentation broth” according to this disclosure mean harvesting, collecting or retrieving the oligosaccharide solution from the cells and/or the medium of its growth. The term “cultivation” refers to the culture medium wherein the cell is cultivated or cultured or fermented, the cell itself, and the oligosaccharides that are produced by the cell in whole broth, i.e., inside (intracellularly) as well as outside (extracellularly) of the cell.

In case the oligosaccharide solution is still present or partly present in the cells producing the oligosaccharide solution, conventional manners to free or to extract the oligosaccharide solution out of the cells can be used, such as cell destruction using high pH, heat shock, sonication, French press, homogenization, enzymatic hydrolysis, chemical hydrolysis, solvent hydrolysis, detergent, hydrolysis. The culture medium and/or cell extract together and/or separately can then be further used for purifying the oligosaccharide solution from the fermentation broth.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of this disclosure are at least about 50.0%, 55.0%, 60.0%, 65.0%, 70.0%, 75.0%, 80.0% or 85.0% pure, usually at least about 90.0%, 91.0%, 92.0%, 93.0%, 94.0%, 95.0%, 96.0%, 97.0%, 98.0%, or 99.0% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, purity can be determined using methods such as but not limited to thin layer chromatography, gas chromatography, NMR, HPLC, capillary electrophoresis or mass spectroscopy.

A “purified oligosaccharide solution” comprises one oligosaccharide or a mixture of oligosaccharides dissolved in an aqueous medium. An aqueous medium is a solvent comprising water. In some embodiments, the aqueous medium is pure water. In other embodiments, the medium comprises water with a trace amount of one or more organic solvents. In some such embodiments, the medium comprises less than 1%-wt. organic solvent. In some embodiments, the medium comprises less than 0.1%-wt. organic solvent. In some embodiments, the medium comprises less than 0.01%-wt. organic solvent. In some embodiments, the medium comprises less than 0.001%-wt. organic solvent. In some embodiments, the medium comprises less than 0.0001%-wt. organic solvent.

In some embodiments, the oligosaccharide solution comprises a trace amount of one or more organic solvents. In some such embodiments, the purified oligosaccharide solution comprises less than 1%-wt. organic solvent. In some embodiments, the purified oligosaccharide solution comprises less than 0.1%-wt. organic solvent. In some embodiments, the purified oligosaccharide solution comprises less than 0.01%-wt. (percent by weight) organic solvent. In some embodiments, the purified oligosaccharide solution comprises less than 0.001%-wt. organic solvent. In some embodiments, the purified oligosaccharide solution comprises less than 0.0001%-wt. organic solvent.

As used herein a “Brix value” indicates the sugar content of an aqueous solution. A Brix value can be expressed as a percentage (percent Brix) or as “degrees Brix” (degrees Brix). Strictly, a Brix value is the percentage by weight of sucrose in a pure water solution, and so does not apply to solutions comprising other solutes and/or solvents. However, a Brix value is simple to measure, and, therefore, is commonly used in the art as an approximation of the total saccharide content of sugar solutions other than pure sucrose solutions. As used herein, the “Brix value” indicates the combined sugar content of the aqueous solution, when the purified oligosaccharide solution comprises two or more different oligosaccharide.

Techniques for measuring a Brix value are well known in the art. Dissolution of sugar in an aqueous solution changes the refractive index of the solution. Accordingly, an appropriately calibrated refractometer can be used to measure a Brix value of a solution.

Alternatively, the density of a solution may be measured and converted to a Brix value. A digital density meter can perform this measurement and conversion automatically, or a hydrometer or pycnometer may be used.

As used herein, the term “bulk density” is the weight of the particles of a particulate solid (such as a powder) in a given volume, and is expressed in grams per liter (g/L). The total volume that the particles of a particulate solid occupy depends on the size of the particles themselves and the volume of the spaces between the particles. Entrapped air between and inside the particles also can affect the bulk density. Thus, a particulate solid comprising large, porous particles with large inter-particulate spaces will have a lower bulk density than a particulate solid comprising small, non-porous particles that compact closely together. Bulk density can be expressed in two forms: “loose bulk density” and “tapped bulk density.” Loose bulk density (also known in the art as “freely settled” or “poured” bulk density) is the weight of a particulate solid divided by its volume where the particulate solid has been allowed to settle into that volume of its own accord (e.g., a powder poured into a container).

Closer compaction of a particulate solid within a container may be achieved by tapping the container and allowing the particles to settle more closely together, thereby reducing volume while weight remains the same. Tapping therefore increases bulk density. Tapped bulk density (also known in the art as “tamped” bulk density) is the weight of a particulate solid divided by its volume where the particulate solid has been tapped and allowed to settle into the volume a precise number of times. The number of times the particulate solid has been tapped is typically when stating the tapped bulk density. For example, “100× tapped bulk density” refers to the bulk density of the particulate solid after it has been tapped 100 times.

Techniques for measuring bulk density are well known in the art. Loose bulk density may be measured using a measuring cylinder and weighing scales: the particulate solid is poured into the measuring cylinder and the weight and volume of the particulate solid; weight divided by volume gives the loose bulk density. Tapped bulk density can be measured using the same technique, with the addition of tapping the measuring cylinder a set number of times before measuring weight and volume. Automation of tapping ensures the number, timing and pressure of individual taps is accurate and consistent. Automatic tapping devices are readily available, an example being the Jolting Stampfvolumeter (STAV 203) from J. Englesmann AG.

The term “different oligosaccharides” as used herein refers to oligosaccharides that are structurally distinct.

The terms “dry solid” and “dry matter” as used herein are used interchangeably and are further described in Example 1.

Throughout the application, unless explicitly stated otherwise, a “genetically modified micro-organism” or “metabolically engineered micro-organism” or “genetically modified cell” or “metabolically engineered cell” preferably means respectively, a microorganism or a cell that is genetically modified or metabolically engineered, respectively, for the production of the mixture comprising different oligosaccharides according to this disclosure. In the context of this disclosure, the different oligosaccharides of the mixture as disclosed herein preferably do not occur in the wild type progenitor of the metabolically engineered micro-organism or cell, respectively.

Throughout the application, unless explicitly stated otherwise, the features “synthesize,” “synthesized” and “synthesis” are interchangeably used with the features “produce,” “produced” and “production,” respectively.

### DETAILED DESCRIPTION

According to a first aspect, a process for purification of an oligosaccharide solution in a batch manner or in a continuous manner from a fermentation broth obtained by cell cultivation or microbial fermentation is provided. The cultivation or fermentation broth further comprises biomass, medium components and contaminants. Preferably, the purity of the oligosaccharide solution in the cultivation or fermentation broth is <80% on total dry solid. The cultivation or fermentation broth is applied to the following purification steps: i) clarifying the cultivation or fermentation broth, ii) removing salts and/or medium components from the clarified cultivation or fermentation broth, and iii) preferably concentrating the oligosaccharide solution, thereby obtaining a purified oligosaccharide solution with a purity of ≥80% on total dry solid. The process is characterized in that step ii) of removing salts and/or medium components from the clarified cultivation or fermentation broth comprises an ion exchanger treatment comprising cationic ion exchanger treatment for the removal of positively charged material and that does not comprise an anionic ion exchange treatment for the removal of negatively charged material.

The purification comprises a combination of clarification of the cultivation or fermentation broth and removing salts and/or medium components from the clarified cultivation or fermentation broth and preferably concentrating the oligosaccharide solution. In an embodiment, the clarification is combined with the removal of salts and/or medium components. In an embodiment, the clarification is combined with the step of concentrating the oligosaccharide solution in the clarified cultivation or fermentation broth and thereafter performing step ii). In an embodiment, the clarification is combined with the removal of salts and/or medium components and further combined with the step of concentrating the oligosaccharide solution resulting from the step of removal of salts and/or medium components. In an embodiment, the clarification is combined with the step of concentrating the oligosaccharide solution and further combined with the removal of salts and/or medium components of the oligosaccharide solution resulting from the step of concentrating and further combined with a step of concentration after step ii).

The process of this disclosure allows efficient purification of large quantities of oligosaccharide solution, comprising only one oligosaccharide or a mix of oligosaccharides, at high purity.

Contrary to the purification currently used in cell cultivation or fermentation for the production of oligosaccharides that provide for the separation of oligosaccharides from the cultivation or fermentation broth using ion exchange treatments with both anion and cation exchange, the present method allows the provision of a simplified purification of oligosaccharide solutions by omitting the use anion exchanger treatment. The so purified oligosaccharide solution may be obtained in solid form by drying, spray drying, lyophilization or concentrated to a syrup of at least 40% dry matter. The provided oligosaccharide(s) are free of proteins and recombinant material originating from the used recombinant cell or microbial strains and thus very well-suited for use in food, medical food and feed (e.g., pet food) applications.

In an embodiment, the purification involves clarifying the oligosaccharide solution containing cultivation or fermentation broth to remove suspended particulates and contaminants, particularly cells, cell components, insoluble metabolites and debris produced by culturing the genetically modified cell. In this step, the cultivation or fermentation broth containing the produced oligosaccharide solution can be clarified in a conventional manner. Preferably, the cultivation or fermentation broth is clarified by centrifugation, flocculation, decantation, ultrafiltration and/or filtration. A second step of purifying the oligosaccharide solution from the cultivation or fermentation broth involves removing salts and/or medium components, comprising proteins, as well as peptides, amino acids, RNA and DNA and any endotoxins and glycolipids that could influence purity, from the cultivation or fermentation broth containing the oligosaccharide solution, after it has been clarified. This step comprises an ion exchanger treatment comprising cationic ion exchanger treatment for removal of positively charged material and the ion exchanger treatment does not comprise an anionic ion exchange treatment for the removal of negatively charged material. In this step also proteins, salts, by-products, color and other related impurities are removed from the oligosaccharide solution containing cultivation or fermentation broth by further, nanofiltration, reverse osmosis, microfiltration, activated charcoal or carbon treatment, tangential flow high-performance filtration, tangential flow ultrafiltration, affinity chromatography, further cation exchange, hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while the oligosaccharide solution remains in clarified, and possibly concentrated, cultivation or fermentation broth. A third step of purifying the oligosaccharide solution from the cultivation or fermentation broth preferably involves concentrating the cultivation or fermentation broth. In an embodiment, the third step precedes the second step. In an embodiment, the step of concentrating precedes the second step and is once more applied after the second step as described above.

In an embodiment, the purified oligosaccharide solution has an ash content of ≤10% on total dry solid, preferably ≤9% on total dry solid, more preferably ≤8% on total dry solid, even more preferably ≤7% on total dry solid, even more preferably ≤6% on total dry solid, even more preferably ≤5% on total dry solid, even more preferably ≤4% on total dry solid, even more preferably ≤3% on total dry solid, even more preferably ≤2% on total dry solid, even more preferably ≤1% on total dry solid, most preferably ≤0.5% on total dry solid.

In an embodiment, the oligosaccharide solution is purified from a cultivation or fermentation broth obtained by cell cultivation using at least one cell or a microbial fermentation using at least one micro-organism, respectively. The cell can be a fungal, yeast, bacterial, insect, animal, and plant and protozoan cell. Preferably, the cells used are cells of a micro-organism. The micro-organism is preferably bacteria or yeast.

In an embodiment, the cell is a recombinant cell grown in a chemically defined medium, wherein biomass separated in step i) is optionally recycled to the cell cultivation.

In an embodiment, the cell cultivation is using a recombinant cell and comprises at least one cell that has been genetically modified to produce oligosaccharide, preferably the at least one cell has been genetically modified to produce at least two different oligosaccharides.

In an embodiment, the oligosaccharide solution in the cultivation broth is obtained by cell cultivation using at least one genetically modified cell capable of producing the oligosaccharide solution, preferably from an internalized carbohydrate precursor.

In an embodiment, the micro-organism is a recombinant micro-organism grown in a chemically defined medium, wherein biomass separated in step i) is optionally recycled to the microbial fermentation.

In an embodiment, the microbial fermentation is using a recombinant micro-organism and comprises at least one micro-organism that has been genetically modified to produce oligosaccharide, preferably the at least one micro-organism has been genetically modified to produce at least two different oligosaccharides.

In an embodiment, the oligosaccharide solution in the fermentation broth is obtained by microbial fermentation using at least one genetically modified micro-organism capable of producing the oligosaccharide solution, preferably from an internalized carbohydrate precursor.

According to an embodiment of this disclosure, the cell cultivation or microbial fermentation is cultured in a minimal salt medium with a carbon source on which the at least one cell or micro-organism, respectively, grows. Preferably, the minimal salt medium contains sulphate, phosphate, chloride, ammonium, calcium ion, magnesium ion, sodium ion, potassium ion, iron ion, copper ion, zinc ion, manganese ion, cobalt ion, and/or selenium ion.

The at least one cell or micro-organism as used herein grows on a monosaccharide, disaccharide, oligosaccharide, polysaccharide, polyol, a complex medium or a mixture thereof as the main carbon source. With the term main is meant the most important carbon source for the bioproducts of interest, biomass formation, carbon dioxide and/or by-products formation (such as acids and/or alcohols, such as acetate, lactate, and/or ethanol), i.e., 20, 30, 40, 50, 60, 70, 75, 80, 85, 90, 95, 98, 99% of all the required carbon is derived from the above-indicated carbon source. In an embodiment of this disclosure, the carbon source is the sole carbon source for the organism, i.e., 100% of all the required carbon is derived from the above-indicated carbon source. Common main carbon sources comprise but are not limited to glucose, glycerol, fructose, maltose, lactose, arabinose, malto-oligosaccharides, maltotriose, sorbitol, xylose, rhamnose, sucrose, galactose, mannose, methanol, ethanol, trehalose, starch, cellulose, hemi-cellulose, corn-steep liquor, high-fructose syrup, acetate, citrate, lactate and pyruvate. With the term complex medium is meant a medium for which the exact constitution is not determined. Examples are molasses, corn steep liquor, peptone, tryptone or yeast extract.

Alternatively or preferably, the carbon source comprises one or more of glucose, fructose, mannose, sucrose, maltose, corn steep liquor, lactose, galactose, high fructose syrup, starch, cellulose, hemi-cellulose, malto-oligosaccharides, trehalose, glycerol, acetate, citrate, lactate and pyruvate.

In an embodiment, the purity of the oligosaccharide solution in the cultivation or fermentation broth is <70%, <60%, <50%, <40%, <30%, <20%, <10% on total dry solid, before the purification and/or the purity of the purified oligosaccharide solution is >80%, preferably of >85%, more preferably >90%, even more preferably >95%, most preferably >97% on total dry solid after the purification.

In an embodiment, the yield of the purification of the oligosaccharide solution is >60%, preferably >65%, more preferably >70%, even more preferably >75%. The yield being calculated on the basis of the total mass of oligosaccharide or oligosaccharides in the final syrup or powder divided by the total mass of oligosaccharide or oligosaccharides in the broth after clarification in percentage.

In an embodiment, the oligosaccharide solution comprises only one oligosaccharide to be purified.

In an embodiment, the oligosaccharide solution comprises at least 2 different oligosaccharides, preferably at least 3 different oligosaccharides, more preferably at least 4 different oligosaccharides, even more preferably at least 5 different oligosaccharides, most preferably at least 6 different oligosaccharides.

Such an oligosaccharide solution comprising different oligosaccharides can comprise, for example, 5 structurally different oligosaccharides such as e.g., 2′-FL, 3-FL, LNT, 3′-SL and 6′-SL; another example comprises 7 structurally different oligosaccharides such as e.g., 2′-FL, 3-FL, LNT, LNnT, LNFPI, 3′-SL and 6′-SL.

In another preferred embodiment, the oligosaccharide solution comprises at least 2 different oligosaccharides that differ in degree of polymerization (DP), preferably the oligosaccharide solution comprises at least 3 different oligosaccharides that differ in degree of polymerization, more preferably the oligosaccharide solution comprises at least 4 different oligosaccharides that differ in degree of polymerization. The degree of polymerization of an oligosaccharide refers to the number of monosaccharide units present in the oligosaccharide structure. As used herein, the degree of polymerization of an oligosaccharide is three (DP3) or more, the latter comprising any one of 4 (DP4), 5 (DP5), 6 (DP6) or longer. The oligosaccharide solution as described herein preferably comprises at least three different oligosaccharides wherein all oligosaccharides present in the solution have a different degree of polymerization from each other. For example, the oligosaccharide solution can comprise three oligosaccharides, wherein the first oligosaccharide is a trisaccharide with a degree of polymerization of 3 (DP3), the second oligosaccharide is a tetrasaccharide with a degree of polymerization of 4 (DP4) and the third oligosaccharide is a pentasaccharide with a degree of polymerization of 5 (DP5).

In an embodiment, an oligosaccharide solution comprising two different oligosaccharides that differ in degree of polymerization is a mixture of 2′FL and LNT; mix of 2′FL and DiFL or a mixture of 2′FL and LNFPI. In an embodiment an oligosaccharide mixture comprising three different oligosaccharides that differ in DP is a mixture of 2′FL, DiFL and LNFPI.

In an embodiment, the at least one cell produces an oligosaccharide solution comprising four different oligosaccharides or more than four different oligosaccharides. Such solution can comprise at least four different oligosaccharides wherein three of the oligosaccharides have a different degree of polymerization. Alternatively, all of the oligosaccharides in the solution can have a different degree of polymerization as described herein.

Alternatively, or preferably, the oligosaccharide solution comprises at least one neutral and at least one charged oligosaccharide.

According to an aspect of this disclosure, the step i) of clarifying the cultivation or fermentation broth comprises one or more of clarification, clearing, filtration, microfiltration, centrifugation, decantation and ultrafiltration, preferably the step i) further comprising use of a filter aid and/or flocculant. Alternatively, or preferably, step i) comprises subjecting the cultivation or fermentation broth to two membrane filtration steps using different membranes. Further alternatively or preferably, step i) of clarifying the cultivation or fermentation broth further comprises use of a filtration aid, preferably an adsorbing agent, more preferably active carbon.

In an aspect of this disclosure, step ii) of removing salts and/or medium components from the clarified cultivation or fermentation broth comprises an ion exchanger treatment comprising cationic ion exchanger treatment for removal of positively charged material and the ion exchanger treatment does not comprise an anionic ion exchange treatment for the removal of negatively charged material. Step ii) preferably further comprises at least one or more of nanofiltration, dialysis, electrodialysis, use of activated charcoal or carbon, use of solvents, use of alcohols, and use of aqueous alcohol mixtures, use of charcoal, tangential flow high-performance filtration, tangential flow ultrafiltration, affinity chromatography, cation exchange, simulated moving bed chromatography, hydrophobic interaction chromatography, gel filtration, ligand exchange chromatography, column chromatography, cation exchange adsorbent resin, and use of cation exchange resin.

In another aspect, step iii) of concentrating comprises one or more of nanofiltration, diafiltration, reverse osmosis, evaporation, wiped film evaporation, and falling film evaporation.

In an embodiment, the oligosaccharide solution comprises at least one of a fucosylated oligosaccharide, sialylated oligosaccharide, Lewis type antigen, an N-acetylglucosamine containing neutral oligosaccharide, N-acetyllactosamine containing oligosaccharide, lacto-N-biose containing oligosaccharide, non-fucosylated neutral oligosaccharide, chitosan, chitosan oligosaccharide, heparosan, chondroitin sulphate, glycosaminoglycan oligosaccharide, heparin, heparan sulphate, chondroitin sulphate, dermatan sulphate, hyaluronan or hyaluronic acid and/or keratan sulphate. Alternatively or preferably, the oligosaccharide solution comprises a mammalian milk oligosaccharide, preferably a human milk oligosaccharide (HMO).

In an embodiment, the oligosaccharide solution comprises a neutral HMO as defined herein, preferably selected from the group comprising 2′-fucosyl lactose, 3-fucosyl lactose, 2′,3-difucosyllactose, lacto-N-triose II, lacto-N-tetraose, lacto-N-neotetraose, lacto-N-fucopentaose I, lacto-N neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, step i) comprises a first step of clarification by microfiltration. Alternatively, step i) comprises a first step of clarification by centrifugation, a first step of clarification by flocculation, or a first step of clarification by ultrafiltration.

In some embodiments, step i) comprises ultrafiltration.

Preferably, the ultrafiltration in step i) has a molecular weight cut-off equal to or higher than 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa. Alternatively or preferably, step i) comprises two consecutive ultrafiltrations, and wherein the membrane molecular weight cut-off of the first ultrafiltration is higher than that of the second ultrafiltration.

In a preferred embodiment, step ii) comprises nanofiltration and/or electrodialysis. Preferably, the nanofiltration and/or electrodialysis is performed twice. More preferably, the nanofiltration and/or electrodialysis steps are performed consecutively.

In some embodiments, the ultrafiltration permeate of step i) is nanofiltered and/or electrodialysed in step ii).

In some embodiments, step i) is ultrafiltration, and step ii) is nanofiltration and/or electrodialysis treatment combined with a cation exchange treatment.

In an embodiment, step i) is ultrafiltration, the step ii) is nanofiltration and/or electrodialysis treatment combined with a cation exchange treatment, wherein the cation exchange treatment is preceded by ultrafiltration followed by nanofiltration and/or electrodialysis.

In some embodiments using ultrafiltration in step i) and nanofiltration in step ii), preferably the nanofiltration membrane has a molecular weight cut-off that is lower than that of the ultrafiltration membrane in step i).

As used herein, the molecular weight cut-off of the nanofiltration membrane in step ii) is preferably equal to or higher than 200 Da. Such as 200 Da, 300 Da, 400 Da, 500 Da, 600 Da, 700 Da, 800 Da, 900 Da, or 1000 Da. Preferably between 300 and 500 Da and/or between 600 and 800 Da.

In an embodiment, the cationic ion exchanger treatment is a strongly acidic cation exchanger treatment, preferably treatment with a strong cation exchange resin in H+ form, K+ or Na+ form.

In an embodiment, the pH of the eluent of the cation exchange treatment is controlled to keep the pH between 4 and 7, preferably by means of phosphoric acid, sulphuric acid, acetic acid, lactic acid, citric acid, tartric acid, malic acid, succinic acid, fumaric acid.

In some embodiments, the process does not comprise electrodialysis.

In some embodiments, the process does comprise electrodialysis.

In a specific embodiment of this disclosure, one or more steps i) to iii) are performed more than once.

In an embodiment, the cation exchange treatment is a cation exchange resin, preferably on a neutral solid phase.

In an embodiment, after at least one of the purification steps i) or ii); the oligosaccharide solution is diafiltered and/or concentrated, preferably with a nanofiltration membrane, more preferably with a nanofiltration membrane having a size exclusion limit of less than or equal to 20 A, wherein most preferably the solution is diafiltered until a conductivity of less than or equal to 15 mS/cm, preferably less than or equal to 10 mS/cm, more preferably less than or equal to 5 mS/cm, is reached.

The method of this disclosure provides preferably a solution comprising the purified oligosaccharide solution with a Brix value of from about 8 to about 75%, preferably the solution comprising the purified oligosaccharide solution has a Brix value of from about 30 to about 65%.

In an embodiment, the purified oligosaccharide solution contains at least a saccharide amount of at least 20.0% (w/v), 30.0% (w/v), 35.0% (w/v), and up to 45.0% (w/v), 50.0% (w/v), 60.0% (w/v).

In an embodiment, the purified oligosaccharide solution is sterile filtered and/or subjected to endotoxin removal, preferably by filtration of the purified oligosaccharide solution through a 3 kDa filter.

Further according to a preferred embodiment of this disclosure, step i) is preceded by an enzymatic treatment. Preferably, the enzymatic treatment comprises incubation of the cultivation or fermentation broth with one or more enzymes selected from the group comprising: glycosidase, lactase, b-galactosidase, fucosidase, sialidase, maltase, amylase, hexaminidase, glucuronidase, trehalase, and invertase. In an embodiment, the enzymatic treatment converts lactose and/or sucrose to monosaccharides.

According to preferred embodiment, the method further comprises decolorization.

In an embodiment, the purified oligosaccharide solution has an ash content below 10% (on total dry solid) with a Lead content lower than 0.1 mg/kg dry solid, more preferably lower than 0.05 mg/kg dry solid, even more preferably below 0.02 mg/kg dry solid; Arsenic content lower than 0.2 mg/kg dry solid, more preferably lower than 0.1 mg/kg, even more preferably lower than 0.05 mg/kg dry solid, Cadmium content lower than 0.1 mg/kg dry solid, more preferably lower than 0.05 mg/kg dry solid, even more preferably below 0.02 mg/kg dry solid; and/or Mercury content lower than 0.5 mg/kg dry solid, more preferably lower than 0.2 mg/kg dry solid, even more preferably below 0.1 mg/kg.

In an embodiment, the purified oligosaccharide solution has a protein content below 100 mg per kg dry solid, DNA content below 10 ng per gram dry solid and/or endotoxin content below 10000 EU per gram dry solid. A protein content below 100 mg per kg dry solid is preferably below 100 mg, below 90 mg, below 80 mg, below 70 mg, below 60 mg, below 50 mg, below 40 mg, below 30 mg, below 20 mg, below 10 mg, below 5 mg per kg dry solid. A DNA content below 10 ng per gram dry solid is preferably below 10 ng, below 9 ng, below 8 ng, below 7 ng, below 6 ng, below 5 ng, below 4 ng, below 3 ng, below 2 ng, below 1 ng per gram dry solid. An endotoxin content below 10000 EU per gram dry solid is preferably below 7500 EU, below 5000 EU, below 2500 EU, below 1000 EU, below 750 EU, below 500 EU, below 250 EU, below 100 EU, below 50 EU per gram dry solid.

In an embodiment, the purified oligosaccharide solution is free of DNA, proteins, and/or recombinant genetic material.

Another aspect of this disclosure provides for a method wherein the at least one cell is a fungal, yeast, bacterial, insect, animal, and plant and protozoan cell. Preferably, the cells used are cells of a micro-organism. Another aspect of this disclosure provides for a method wherein the at least one micro-organism is a fungal, yeast or bacterial cell as described herein. The at least one micro-organism is chosen from the list comprising a bacterium, a yeast, or a fungus. The latter bacterium preferably belongs to the phylum of the Proteobacteria or the phylum of the Firmicutes or the phylum of the Cyanobacteria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, this disclosure specifically relates to a mutated and/or transformed Escherichia coli cell or strain as indicated above wherein the E. coli strain is a K12 strain. More preferably, the Escherichia coli K12 strain is E. coli MG1655.

The latter bacterium belonging to the phylum Firmicutes belongs preferably to the Bacilli, preferably Lactobacilliales, with members such as Lactobacillus lactis, Leuconostoc mesenteroides, or Bacillales with members such as from the genus Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae.

The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Candida, Hansenula, Kluyveromyces, Pichia, Schizosaccharomyces, Schwanniomyces, Torulaspora, Yarrowia, and Zygosaccharomyces; preferably selected from the group comprising: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii.

The latter fungus belongs preferably to the genus Rhizopus, Dictyostelium, Penicillium, Mucor or Aspergillus.

In an embodiment, the at least one micro-organism is an E. coli or yeast of lactose permease positive phenotype wherein the lactose permease is coded by the gene LacY or LAC12, respectively.

In an embodiment, the purified oligosaccharide solution is further concentrated to a syrup of at least 40% dry matter, the purified oligosaccharide solution is crystalized or the purified oligosaccharide solution is dried to a powder.

In an embodiment, step iii) comprises using vacuum evaporation or reverse osmosis or nanofiltration a) to an oligosaccharide concentration of >100 g/L, preferably >200 g/L, more preferably >300 g/L, more preferably >400 g/L, more preferably >500 g/L, more preferably >600 g/L, most preferably between 300 g/L and 650 g/L; and/or b) at a temperature of <60° C., preferably <50° C., more preferably 20° C. to 50° C., even more preferably 30° C. to 45° C., during vacuum evaporation or reverse osmosis; and/or c) at a temperature of <80° C., preferably <50° C., more preferably 20° C. to 50° C.

In an embodiment, the purified oligosaccharide solution comprises one oligosaccharide and is concentrated to a concentration of >1.5 M and cooled to a temperature <25° C., more preferable <8° C., to obtain crystalline material of the oligosaccharide.

In an embodiment, the purified oligosaccharide solution is dried.

In an embodiment, the step of drying comprises any one or more of spray drying, lyophilization, evaporation, precipitation, spray freeze drying, freeze spray drying, band drying, belt drying, vacuum band drying, vacuum belt drying, drum drying, vacuum drum drying, roller drying, vacuum roller drying and other types of drying. In an embodiment, the purified oligosaccharide solution is spray-dried.

In an embodiment, drying is spray-drying or freeze-drying the purified oligosaccharide solution, preferably at a pH of the solution lower than 5.0.

In an embodiment, the purified solution is spray-dried, particularly spray-dried at an oligosaccharide solution concentration of 20-60 (w/v), preferably 30-50 (w/v), more preferably 35-45 (w/v), a nozzle temperature of 110-150° C., preferably 120-140° C., more preferably 125-135° C. and/or an outlet temperature of 60-80° C., preferably 65-70° C.

In some embodiments, the purified oligosaccharide solution has a Brix value of from about 8 to about 75 percent Brix before drying, e.g., spray drying or lyophilization. In some embodiments, the purified oligosaccharide solution has a Brix value of from about 30 to about 65 percent Brix before drying. In some embodiments, the purified oligosaccharide solution has a Brix value of from about 50 to about 60 percent Brix before drying, preferably spray drying. In some embodiments, the purified oligosaccharide solution has a Brix value of about 50 percent Brix before drying.

In some embodiments, the purified oligosaccharide solution is spray dried. In some embodiments, the purified oligosaccharide solution feed into the spray dryer has a Brix value of from about 8 to about 75 percent Brix. In some embodiments, purified oligosaccharide solution feed into the spray dryer has a Brix value from about 30 to about 65 percent Brix. In some embodiments, purified oligosaccharide solution feed into the spray dryer has a Brix value from about 50 to about 60 percent Brix.

In some embodiments, the feed into the spray dryer is at a temperature of from about 2 to about 70 degrees centigrade immediately before being dispersed into droplets in the spray dryer. In some embodiments, the feed into the spray dryer is at a temperature of from about 30 to about 60 degrees centigrade immediately before being dispersed into droplets in the spray dryer. In some embodiments, the feed into the spray dryer is at a temperature of from about 2 to about 30 degrees centigrade immediately before being dispersed into droplets in the spray dryer. In some embodiments, the spray drying uses air having an air inlet temperature of from 120 to 280 degrees centigrade In some embodiments, the air inlet temperature is from 120 to 210 degrees centigrade In some embodiments, the air inlet temperature is from about 130 to about 190 degrees centigrade In some embodiments, the air inlet temperature is from about 135 to about 160 degrees centigrade In some embodiments, the spray drying uses air having an air outlet temperature of from about 80 to about 110 degrees centigrade In some embodiments, the air outlet temperature is from about 100 to about 110 degrees centigrade In some embodiments, the spray drying is carried out at a temperature of from about 20 to about 90 degrees centigrade In some embodiments, the spray dryer is a co-current spray dryer. In some embodiments, the spray dryer is attached to an external fluid bed. In some embodiments, the spray dryer comprises a rotary disk, a high pressure nozzle or a two-fluid nozzle. In some embodiments, the spray dryer comprises an atomizer wheel. In some embodiments, spray-drying is the final purification step for the purified oligosaccharide solution.

In an embodiment, the final purification step for the purified oligosaccharide solution is lyophilization.

In an embodiment, the final purification step for the purified oligosaccharide solution is concentration to a syrup.

A further aspect provides a purified oligosaccharide solution, oligosaccharide or oligosaccharide mixture obtained according to the process described herein.

An embodiment provides an oligosaccharide producible with the process described herein, wherein the oligosaccharide solution is preferably spray-dried, lyophilized or crystallized.

An embodiment provides an oligosaccharide mixture producible with the process described herein, wherein the oligosaccharide solution is preferably spray-dried, lyophilized or concentrated to a syrup of at least 40% dry matter.

An embodiment provides an oligosaccharide solution produced through cultivation or fermentation wherein the oligosaccharide solution is purified without anion exchange and wherein the oligosaccharide solution contains less than 10% ash.

An embodiment provides a spray dried oligosaccharide or oligosaccharide mixture produced through cultivation or fermentation wherein the oligosaccharide or oligosaccharide mixture is purified without anion exchange and wherein the spray dried oligosaccharide or oligosaccharide mixture contains less than 10% ash.

In an embodiment, the dried powder contains a low amount of water.

Techniques for measuring moisture content of a material are well known in the art. Examples include Karl-Fischer titration, wherein the quantity of Karl-Fischer solution absorbed by a sample indicates the amount of water in the sample, and gravimetric methods, wherein a sample is dried and weight loss due to evaporation of solvent is measured at intervals.

An embodiment provides a dried powder obtained according to any one of the process of embodiment 56 to 60, wherein the dried powder contains ≤15%-wt. of water, preferably ≤10%-wt. of water, more preferably ≤7%-wt. of water, most preferably ≤5%-wt. of water. In an additional and/or alternative embodiment, the dried powder is free of genetically-engineered microorganisms and free of nucleic acid molecules derived from genetically-engineered microorganisms.

In a specific embodiment, this disclosure provides the purified oligosaccharide solution that is spray-dried to powder, wherein the spray-dried powder contains ≤15%-wt. of water, preferably ≤10%-wt. of water, more preferably ≤7%-wt. of water, most preferably ≤5%-wt. of water. In some embodiments, the spray dryer is operated to achieve a moisture content of from about 3.0 to 5.0%-wt. of water. In some embodiments, the spray-dried oligosaccharide solution has a moisture content of less than 5%-wt. of water. In some embodiments, the spray-dried oligosaccharide solution has a moisture content of less than about 2.3 percent (by weight) water.

This specification provides spray dried powder obtainable according to the process described herein, wherein the powder has a mean particle size of 50 to 250 μm determined by laser diffraction, preferably the powder has a mean particle size of 95 to 120 μm determined by laser diffraction; more preferably the powder has a mean particle size of 110 to 120 μm. Such particle sizes are dependent on the dryer's specifications, configuration, performance and design. Commercial driers available are, for example, the Büchi mini spray dryer, Procept spray dryers, Gea spray dryers, optionally with either rotary atomizer, two-fluid nozzle, pressure nozzle, combi-nozzle, optionally in open-mode design, multi-stage drying design, closed-cycle design. The above particle sizes where obtained using Buchi spray dryer (Buchi Mini Spray Dryer B-290) (Büchi, Essen, Germany), applying the following parameters: Inlet—temperature: 130° C., Outlet temperature 67° C.-71° C., gasflow 670 L/h, aspirator 100%.

This specification also provides dried powder obtainable according to the process described herein, wherein the dried powder is having a loose bulk density of from about 500 to about 700 g/L, a 100× tapped bulk density of from about 600 to about 850 g/L, a 625× tapped bulk density of from about 600 to about 900 g/L and/or a 1250× tapped bulk density of from about 650 to about 900 g/L.

In some embodiments, the dried powder has a loose bulk density of from about 600 to about 700 g/L. In some embodiments, the dried powder has a loose bulk density of from about 500 to about 600 g/L.

In some embodiments, the dried powder has a 100× tapped bulk density of from about 750 to about 850 g/L. In some embodiments, the dried powder has 100× tapped bulk density of from about 600 to about 700 g/L.

In some embodiments, the dried powder has a 625× tapped bulk density of from about 750 to about 900 g/L. In some embodiments, the dried powder has a 625× tapped bulk density of from about 700 to about 800 g/L.

In some embodiments, the dried powder has a 1250× tapped bulk density of from about 850 to about 900 g/L. In some embodiments, the dried powder has a 1250× tapped bulk density of from about 750 to about 800 g/L.

In some embodiments, the dried powder has a loose bulk density of from about 600 to about 700 g/L, a 100× tapped bulk density of from about 750 to about 850 g/L, a 625× tapped bulk density of from about 750 to about 850 g/L and/or a 1250× tapped bulk density of from about 850 to about 900 g/L. In some embodiments, the dried powder has a loose bulk density of from about 500 to about 600 g/L, a 100× tapped bulk density of from about 600 to about 700 g/L, a 625× tapped bulk density of from about 700 to about 800 g/L and/or a 1250× tapped bulk density of from about 750 to about 800 g/L.

In an embodiment, the dried oligosaccharide solution when redissolved in water at a concentration of 10% (mass on volume) provides a solution with a pH between 4 and 7, preferably with a pH between 4 and 6.

An embodiment provides an oligosaccharide as described herein and as produced herein, wherein the oligosaccharide is a mammalian milk oligosaccharide, preferably a human milk oligosaccharide (HMO). An embodiment provides an oligosaccharide mixture as described herein, wherein the mixture comprises a mammalian milk oligosaccharide, preferably a human milk oligosaccharide (HMO).

An embodiment provides human milk oligosaccharide (HMO) as described herein and as produced herein, wherein the HMO is a neutral HMO selected from the group comprising 2′-fucosyl lactose, 3-fucosyl lactose, 2′,3-difucosyllactose, lacto-N-triose II, lacto-N-tetraose, lacto-N-neotetraose, lacto-N-fucopentaose I, lacto-N neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

An embodiment provides oligosaccharide or oligosaccharide mixture as described herein, wherein the oligosaccharide or oligosaccharide mixture a) has a conductivity of less than 1 mS/cm at a 300 g/l solution; b) is free of recombinant DNA material, optionally free of any DNA; and/or c) is free of proteins derived from the recombinant micro-organism, optionally free of any proteins.

An embodiment provides oligosaccharide or oligosaccharide mixture as described herein for use in medicine, preferably for use in prophylaxis or therapy of a gastrointestinal disorder.

Oligosaccharide Mix Compositions

In an embodiment, the oligosaccharide mixture contains 2′FL, 3FL, and LDFT wherein the relative percentage of 2′FL to the sum of the masses of 2′FL, 3FL and LDFT is between 65% and 79%, the relative percentage of 3FL to the sum of the masses of 2′FL, 3FL and LDFT is between 17% and 21% and the relative percentage of LDFT to the sum of the masses of 2′FL, 3FL and LDFT is between 9% and 10%.

In an embodiment, the oligosaccharide mixture contains 2′FL, 3FL, and LDFT wherein the relative percentage of 2′FL to the sum of the masses of 2′FL, 3FL and LDFT is between 10% and 12%, the relative percentage of 3FL to the sum of the masses of 2′FL, 3FL and LDFT is between 79% and 96% and the relative percentage of LDFT to the sum of the masses of 2′FL, 3FL and LDFT is between 1% and 2%.

In an embodiment, the oligosaccharide mixture contains 2′FL, 3FL, LDFT, 3′SL and 6′SL wherein the relative percentage of 2′FL to the sum of the masses of 2′FL, 3FL, LDFT, 3′SL and 6′SL is between 53% and 64%, the relative percentage of 3FL to the sum of the masses of 2′FL, 3FL, LDFT, 3′SL and 6′SL is between 14% and 17% and the relative percentage of LDFT to the sum of the masses of 2′FL, 3FL, LDFT, 3′SL and 6′SL is between 7% and 8% and the relative percentage of 3′SL to the sum of the masses of 2′FL, 3FL, LDFT, 3′SL and 6′SL is between 5% and 6% and the relative percentage of 6′SL to the sum of the masses of 2′FL, 3FL, LDFT, 3′SL and 6′SL is between 12% and 15%.

In an embodiment, the oligosaccharide mixture contains LNT, LNnT, 3′SL and 6′SL wherein the relative percentage of LNT to the sum of the masses of LNT, LNnT, 3′SL and 6′SL is between 48% and 59%, the relative percentage of LNnT to the sum of the masses of LNT, LNnT, 3′SL and 6′SL is between 13% and 16% and the relative percentage of 3′SL to the sum of the masses of LNT, LNnT, 3′SL and 6′SL is between 8% and 10% and the relative percentage of 6′SL to the sum of the masses of LNT, LNnT, 3′SL and 6′SL is between 20% and 25%.

In an embodiment, the oligosaccharide mixture contains 3′SL and 6′SL wherein the relative percentage of 3′SL to the sum of the masses of 3′SL and 6′SL is between 26% and 32% and the relative percentage of 6′SL to the sum of the masses of 3′SL and 6′SL is between 64% and 78%.

In an embodiment, the oligosaccharide mixture contains 3′SL and 6′SL wherein the relative percentage of 6′SL to the sum of the masses of 3′SL and 6′SL is between 26% and 32% and the relative percentage of 3′SL to the sum of the masses of 3′SL and 6′SL is between 64% and 78%.

In an embodiment, the oligosaccharide mixture contains 2′FL, 3FL, LDFT, LNFP I, LNFP II, LNFP III and LNFP V wherein the relative percentage of 2′FL to the sum of the masses 2′FL, 3FL, LDFT, LNFP I, LNFP II, LNFP III and LNFP V is between 38% and 46%, the relative percentage of 3FL to the sum of the masses of 2′FL, 3FL, LDFT, LNFP I, LNFP II, LNFP III and LNFP V is between 10% and 12% and the relative percentage of LDFT to the sum of the masses of 2′FL, 3FL, LDFT, LNFP I, LNFP II, LNFP III and LNFP V is between 5% and 6% and the relative percentage of LNFP I to the sum of the masses of 2′FL, 3FL, LDFT, LNFP I, LNFP II, LNFP III and LNFP V is between 21% and 25% and the relative percentage of LNFP II to the sum of the masses of 2′FL, 3FL, LDFT, LNFP I, LNFP II, LNFP III and LNFP V is between 10% and 13%, and the relative percentage of LNFP III to the sum of the masses of 2′FL, 3FL, LDFT, LNFP I, LNFP II, LNFP III and LNFP V is between 5% and 6% and the relative percentage of LNFP V to the sum of the masses of 2′FL, 3FL, LDFT, LNFP I, LNFP II, LNFP III and LNFP V is between 1% and 2%.

In an embodiment, the oligosaccharide mixture contains 2′FL, 3FL, LDFT, LNFP I, LNFP II, LNFP III and LNFP V wherein the relative percentage of 2′FL to the sum of the masses 2′FL, 3FL, LDFT, LNFP I, LNFP II, LNFP III and LNFP V is between 6% and 8%, the relative percentage of 3FL to the sum of the masses of 2′FL, 3FL, LDFT, LNFP I, LNFP II, LNFP III and LNFP V is between 51% and 63% and the relative percentage of LDFT to the sum of the masses of 2′FL, 3FL, LDFT, LNFP I, LNFP II, LNFP III and LNFP V is between 0.5% and 2% and the relative percentage of LNFP I to the sum of the masses of 2′FL, 3FL, LDFT, LNFP I, LNFP II, LNFP III and LNFP V is between 3% and 5% and the relative percentage of LNFP II to the sum of the masses of 2′FL, 3FL, LDFT, LNFP I, LNFP II, LNFP III and LNFP V is between 17% and 21%, and the relative percentage of LNFP III to the sum of the masses of 2′FL, 3FL, LDFT, LNFP I, LNFP II, LNFP III and LNFP V is between 8% and 10% and the relative percentage of LNFP V to the sum of the masses of 2′FL, 3FL, LDFT, LNFP I, LNFP II, LNFP III and LNFP V is between 2% and 3%.

In an embodiment, the oligosaccharide mixture contains LSTa, LSTb, and LSTc wherein the relative percentage of LSTa to the sum of the masses of LSTa, LSTb, and LSTc is between 15% and 18% and the relative percentage of LSTb to the sum of the masses of LSTa, LSTb, and LSTc is between 13% and 16%, and the relative percentage of LSTc to the sum of the masses of LSTa, LSTb, and LSTc is between 62% and 75%

In an embodiment, the oligosaccharide mixture contains 3′SL, 6′SL, LSTa, LSTb, and LSTc wherein the relative percentage of 3′SL to the sum of the masses of 3′SL, 6′SL, LSTa, LSTb, and LSTc is between 14% and 17% and the relative percentage of 6′SL to the sum of the masses of 3′SL, 6′SL, LSTa, LSTb, and LSTc between 35% and 43% and the relative percentage of LSTa to the sum of the masses of 3′SL, 6′SL, LSTa, LSTb, and LSTc between 7% and 9% and the relative percentage of LSTb to the sum of the masses of 3′SL, 6′SL, LSTa, LSTb, and LSTc between 6% and 8% and the relative percentage of LSTc to the sum of the masses of 3′SL, 6′SL, LSTa, LSTb, and LSTc between 28% and 34%.

In an embodiment, the oligosaccharide mixture contains 2′FL, 3FL, LDFT, LNFP I, LNFP II, LNFP III, LNFP V, 3′SL, 6′SL, LSTa, LSTb, and LSTc wherein the relative percentage of 2′FL to the sum of the masses 2′FL, 3FL, LDFT, LNFP I, LNFP II, LNFP III, LNFP V, 3′SL, 6′SL, LSTa, LSTb, and LSTc is between 20% and 30%, and the relative percentage of 3FL to the sum of the masses 2′FL, 3FL, LDFT, LNFP I, LNFP II, LNFP III, LNFP V, 3′SL, 6′SL, LSTa, LSTb, and LSTc is between 5% and 10%, and the relative percentage of LDFT to the sum of the masses 2′FL, 3FL, LDFT, LNFP I, LNFP II, LNFP III, LNFP V, 3′SL, 6′SL, LSTa, LSTb, and LSTc is between 3% and 6%, and the relative percentage of 3′SL to the sum of the masses 2′FL, 3FL, LDFT, LNFP I, LNFP II, LNFP III, LNFP V, 3′SL, 6′SL, LSTa, LSTb, and LSTc is between 2% and 4%, and the relative percentage of 6′SL to the sum of the masses 2′FL, 3FL, LDFT, LNFP I, LNFP II, LNFP III, LNFP V, 3′SL, 6′SL, LSTa, LSTb, and LSTc is between 5% and 10%, and the relative percentage of LNT to the sum of the masses 2′FL, 3FL, LDFT, LNFP I, LNFP II, LNFP III, LNFP V, 3′SL, 6′SL, LSTa, LSTb, and LSTc is between 11% and 20%, and the relative percentage of LNnT to the sum of the masses 2′FL, 3FL, LDFT, LNFP I, LNFP II, LNFP III, LNFP V, 3′SL, 6′SL, LSTa, LSTb, and LSTc is between 2% and 4%, and the relative percentage of LNFP I to the sum of the masses 2′FL, 3FL, LDFT, LNFP I, LNFP II, LNFP III, LNFP V, 3′SL, 6′SL, LSTa, LSTb, and LSTc is between 12% and 20%, %, and the relative percentage of LNFP II to the sum of the masses 2′FL, 3FL, LDFT, LNFP I, LNFP II, LNFP III, LNFP V, 3′SL, 6′SL, LSTa, LSTb, and LSTc is between 5% and 10%, and the relative percentage of LNFP III to the sum of the masses 2′FL, 3FL, LDFT, LNFP I, LNFP II, LNFP III, LNFP V, 3′SL, 6′SL, LSTa, LSTb, and LSTc is between 3% and 6%, and the relative percentage of LNFP V to the sum of the masses 2′FL, 3FL, LDFT, LNFP I, LNFP II, LNFP III, LNFP V, 3′SL, 6′SL, LSTa, LSTb, and LSTc is between 0.5% and 2%, and the relative percentage of LSTa to the sum of the masses 2′FL, 3FL, LDFT, LNFP I, LNFP II, LNFP III, LNFP V, 3′SL, 6′SL, LSTa, LSTb, and LSTc is between 0.5% and 2%, and the relative percentage of LSTb to the sum of the masses 2′FL, 3FL, LDFT, LNFP I, LNFP II, LNFP III, LNFP V, 3′SL, 6′SL, LSTa, LSTb, and LSTc is between 0.5% and 2%, and the relative percentage of LSTc to the sum of the masses 2′FL, 3FL, LDFT, LNFP I, LNFP II, LNFP III, LNFP V, 3′SL, 6′SL, LSTa, LSTb, and LSTc is between 4% and 8%.

In an embodiment, the oligosaccharide mixture contains 2′FL, 3FL, LDFT, 3′SL, 6′SL, LNT and LNnT wherein the relative percentage of 2′FL to the sum of the masses of 2′FL, 3FL, LDFT, 3′SL, 6′SL, LNT and LNnT is between 37% and 46%, the relative percentage of 3FL to the sum of the masses of 2′FL, 3FL, LDFT, 3′SL, 6′SL, LNT and LNnT is between 10% and 12% and the relative percentage of LDFT to the sum of the masses of 2′FL, 3FL, LDFT, 3′SL, 6′SL, LNT and LNnT is between 4% and 8% and the relative percentage of 3′SL to the sum of the masses of 2′FL, 3FL, LDFT, 3′SL, 6′SL, LNT and LNnT is between 2% and 5% and the relative percentage of 6′SL to the sum of the masses of 2′FL, 3FL, LDFT, 3′SL, 6′SL, LNT and LNnT is between 8% and 10% and the relative percentage of LNT to the sum of the masses of 2′FL, 3FL, LDFT, 3′SL, 6′SL, LNT and LNnT is between 20% and 25% and the relative percentage of LNnT to the sum of the masses of 2′FL, 3FL, LDFT, 3′SL, 6′SL, LNT and LNnT is between 5% and 10%.

In an embodiment, the oligosaccharide mixture contains 2′FL, 3FL, LDFT, 3′SL, 6′SL, LNT and LNnT wherein the relative percentage of 2′FL to the sum of the masses of 2′FL, 3FL, LDFT, 3′SL, 6′SL, LNT and LNnT is between 3% and 6%, the relative percentage of 3FL to the sum of the masses of 2′FL, 3FL, LDFT, 3′SL, 6′SL, LNT and LNnT is between 35% and 46% and the relative percentage of LDFT to the sum of the masses of 2′FL, 3FL, LDFT, 3′SL, 6′SL, LNT and LNnT is between 0.5% and 2% and the relative percentage of 3′SL to the sum of the masses of 2′FL, 3FL, LDFT, 3′SL, 6′SL, LNT and LNnT is between 2% and 5% and the relative percentage of 6′SL to the sum of the masses of 2′FL, 3FL, LDFT, 3′SL, 6′SL, LNT and LNnT is between 8% and 15% and the relative percentage of LNT to the sum of the masses of 2′FL, 3FL, LDFT, 3′SL, 6′SL, LNT and LNnT is between 25% and 31% and the relative percentage of LNnT to the sum of the masses of 2′FL, 3FL, LDFT, 3′SL, 6′SL, LNT and LNnT is between 5% and 10%.

The oligosaccharide mixtures contains LNT and LNnT wherein the relative percentage of LNT to the sum of the masses of LNT and LNnT is between 70% and 90%, the relative percentage of LNnT to the sum of the masses of LNT and LNnT is between 10% and 30%.

The oligosaccharide mixtures contains LNT and LNnT wherein the relative percentage of LNT to the sum of the masses of LNT and LNnT is between 10% and 30%, the relative percentage of LNnT to the sum of the masses of LNT and LNnT is between 70% and 90%.

Products comprising an oligosaccharide solution, oligosaccharide or oligosaccharide mixture

In some embodiments, an oligosaccharide solution, oligosaccharide or oligosaccharide mixture purified by a process of this specification is incorporated into nutritional formulations (such as food, drink or feed), food supplements, dietary supplement, digestive health functional foods or other consumable products, intended for use with infants, children, adults or seniors. Other applications comprise oligosaccharide solution, oligosaccharide or oligosaccharide mixture purified by a process of this specification incorporated into pharmaceutical ingredient, cosmetic ingredient or medicine. In some embodiments, the oligosaccharide solution, oligosaccharide or oligosaccharide mixture is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the oligosaccharide solution, oligosaccharide or oligosaccharide mixture purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis (e.g., BB12), B. longum and B. infantis (e.g., Bi-26, Bi-07, Bb-02, EVC001-ActiBif)), Streptococcus species (Streptococcus thermophilus (e.g., TH-4) and Saccharomyces boulardii. In some embodiments, an oligosaccharide solution, oligosaccharide or oligosaccharide mixture purified by a process of this specification is orally administered in combination with such microorganism.

Examples of further ingredients for dietary supplements include disaccharides (such as lactose), monosaccharides (such as glucose and galactose), thickeners (such as gum arabic), acidity regulators (such as trisodium citrate, phosphoric acid, sulphuric acid, acetic acid, lactic acid, citric acid, tartric acid, malic acid, succinic acid, fumaric acid or salts thereof), water, skimmed milk, and flavorings.

In some embodiments, the oligosaccharide solution, oligosaccharide or oligosaccharide mixture is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, an oligosaccharide solution, oligosaccharide or oligosaccharide mixture purified by a process in this specification is included in infant formula to provide nutritional benefits similar to those provided by the oligosaccharides in human breast milk. In some embodiments, the oligosaccharide solution, oligosaccharide or oligosaccharide mixture is mixed with one or more ingredients of the infant formula. Examples of infant formula ingredients include nonfat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, Bb, Bit, C and D), minerals (such as potassium citrate, calcium citrate, magnesium chloride, sodium chloride, sodium citrate and calcium phosphate) and possibly HMOs. Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise nonfat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the oligosaccharide solution, oligosaccharide or oligosaccharide mixture's concentration in the infant formula is approximately the same concentration as the oligosaccharide's concentration generally present in human breast milk. In some embodiments, the concentration of each of the single oligosaccharides in the mixture of oligosaccharides in the infant formula is approximately the same concentration as the concentration of that oligosaccharide generally present in human breast milk.

In some embodiments, the oligosaccharide solution, oligosaccharide or oligosaccharide mixture is incorporated into a feed preparation, wherein the feed is chosen from the list comprising pet food, animal milk replacer, veterinary product, post weaning feed, or creep feed.

The oligosaccharide solution, oligosaccharide or oligosaccharide mixture purified by a process of this specification can be added to a pharmaceutically acceptable carriers such as conventional additives, adjuvants, excipients and diluents (water, gelatin, talc, sugars, starch, gum Arabic, vegetable gums, vegetable oils, polyalkylene glycols, flavoring agents, preservatives, stabilizers, emulsifying agents, lubricants, colorants, fillers, wetting agents, etc.). Suitable carriers are described in the most recent edition of Remington's Pharmaceutical Sciences, a standard reference text in the field. When the oligosaccharide solution, oligosaccharide or oligosaccharide mixture purified by a process of this specification is added to the pharmaceutically acceptable carriers, a dosage in the form of, for example, but not limited to tablets, powders, granules, suspensions, emulsions, infusions, capsules, injections, liquids, elixirs, extracts and tincture can be made. To the above formulas, if needed, probiotics, e.g., lacto bacteria, Bifidobacterium species, prebiotics such as fructooligosaccharides and galactooligosaccharides, proteins from casein, soy-bean, whey or skim milk, carbohydrates such as lactose, saccharose, maltodextrin, starch or mixtures thereof, lipids (e.g., palm olein, sunflower oil, safflower oil) and vitamins and minerals essential in a daily diet can also be further added.

Pharmaceutical compositions comprising the oligosaccharide solution, oligosaccharide or oligosaccharide mixture purified by a process of this specification can be manufactured by means of any usual manner known in the art, e.g., described in the most recent edition of Remington's Pharmaceutical Sciences, a standard reference text in the field.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the. It goes without saying that the abovementioned features and the features that are still to be explained below can be used not only in the respectively specified combinations, but also in other combinations or on their own, without departing from the scope of this disclosure.

This disclosure relates to following specific embodiments:
1. Process for purification of an oligosaccharide solution in a batch manner or in a continuous manner from a cultivation or fermentation broth obtained by cell cultivation or microbial fermentation, the cultivation or fermentation broth further comprising biomass, medium components and contaminants, wherein preferably the purity of the oligosaccharide solution in the cultivation or fermentation broth is <80% on total dry solid, and wherein the cultivation or fermentation broth is applied to the following purification steps:
   i) clarifying the cultivation or fermentation broth,
   ii) removing salts and/or medium components from the clarified cultivation or fermentation broth, and
   iii) preferably concentrating the oligosaccharide solution,
wherein a purified oligosaccharide solution at a purity of ≥80% on total dry solid is provided,
characterized in that step ii) of removing salts and/or medium components from the clarified cultivation or fermentation broth comprises an ion exchanger treatment comprising cationic ion exchanger treatment for the removal of positively charged material and that does not comprise an anionic ion exchange treatment for the removal of negatively charged material.
2. Process according to embodiment 1, wherein step iii) comes before step ii).
3. Process according to any one of embodiment 1 or 2, wherein the purified oligosaccharide solution has an ash content of ≤10% on total dry solid, preferably ≤9% on total dry solid, more preferably ≤8% on total dry solid, even more preferably ≤7% on total dry solid, even more preferably ≤6% on total dry solid, even more preferably ≤5% on total dry solid, even more preferably ≤4% on total dry solid, even more preferably ≤3% on total dry solid, even more preferably ≤2% on total dry solid, even more preferably ≤1% on total dry solid, most preferably ≤0.5% on total dry solid.
4. Process according to any one of embodiment 1 to 3, wherein the oligosaccharide solution is purified from a cultivation or fermentation broth obtained by cell cultivation, using at least one cell, preferably a recombinant cell grown in a chemically defined medium, wherein biomass separated in step i) is optionally recycled to the cultivation.
5. Process according to any one of embodiment 1 to 4, wherein the oligosaccharide solution is purified from a fermentation broth obtained by microbial fermentation using at least one micro-organism, preferably bacteria or yeast, more preferably a recombinant micro-organism grown in a chemically defined medium, wherein biomass separated in step i) is optionally recycled to the microbial fermentation.
6. Process according to any one of embodiment 1 to 5, wherein the cell cultivation is using a recombinant cell and comprises at least one cell that has been genetically modified to produce oligosaccharide, preferably the at least one cell has been genetically modified to produce at least two different oligosaccharides.
7. Process according to any one of embodiment 1 to 6, wherein the cell cultivation is a microbial fermentation using a recombinant micro-organism and comprises at least one micro-organism that has been genetically modified to produce oligosaccharide, preferably the at least one micro-organism has been genetically modified to produce at least two different oligosaccharides.
8. Process according to any one of embodiment 1 to 7, wherein the oligosaccharide solution in the cultivation or fermentation broth is obtained by cell cultivation or microbial fermentation using at least one genetically modified cell capable of producing the oligosaccharide solution, preferably from an internalized carbohydrate precursor.
9. Process according to embodiment 1 to 8, wherein the cell cultivation or microbial fermentation is cultured in a minimal salt medium with a carbon source on which the at least one cell or micro-organism grows.
10. Process according to embodiment 9, wherein the minimal salt medium contains sulphate, phosphate, chloride, ammonium, calcium ion, magnesium ion, sodium, potassium ion, iron ion, copper ion, zinc ion, manganese ion, cobalt ion, and/or selenium ion.
11. Process according to any one of embodiment 9 or 10, wherein the carbon source comprises one or more of glucose, fructose, mannose, sucrose, maltose, corn steep liquor, lactose, galactose, high fructose syrup, starch, cellulose, hemi-cellulose, malto-oligosaccharides, trehalose, glycerol, acetate, citrate, lactate and pyruvate.
12. Process according to any one of embodiment 1 to 11, wherein the purity of the oligosaccharide solution in the cultivation or fermentation broth is <70%, <60%, <50%, <40%, <30%, <20%, <10% on total dry solid, before the purification and/or the purity of the purified oligosaccharide solution is >80%, preferably of >85%, more preferably >90%, even more preferably >95%, most preferably >97% on total dry solid after the purification.
13. Process according to any one of embodiment 1 to 12, wherein the yield of purification of the oligosaccharide solution is >60%, preferably >65%, more preferably >70%, most preferably >75%.
14. Process according to any one of embodiment 1 to 13, wherein the oligosaccharide solution comprises at least 2 different oligosaccharides, preferably at least 3 different oligosaccharides, more preferably at least 4 different oligosaccharides, even more preferably at least 5 different oligosaccharides, most preferably at least 6 different oligosaccharides.
15. Process according to embodiment 14, wherein the oligosaccharide solution comprises at least 2 different oligosaccharides that differ in degree of polymerization, preferably the oligosaccharide solution comprises at least 3 different oligosaccharides that differ in degree of polymerization, more preferably the oligosaccharide solution comprises at least 4 different oligosaccharides that differ in degree of polymerization.
16. Process according to any one of embodiment 14 or 15, wherein the oligosaccharide solution comprises at least one neutral and at least one charged oligosaccharide.
17. Process according to any one of embodiment 1 to 16, wherein the step i) of clarifying the cultivation or fermentation broth comprises one or more of clarification, clearing, filtration, microfiltration, centrifugation, decantation and ultrafiltration, preferably the step i) further comprising use of a filter aid and/or flocculant; preferably the filtration aid is an adsorbing agent, more preferably active carbon.
18. Process according to any one of embodiment 1 to 17, wherein the step ii) of removing salts and/or medium components from the clarified cultivation or fermentation broth further comprises at least one or more of nanofiltration, dialysis, electrodialysis, use of activated charcoal or carbon, use of solvents, use of alcohols, and use of aqueous alcohol mixtures, use of charcoal, tangential flow high-performance filtration, tangential flow ultrafiltration, affinity chromatography, cation exchange, simulated moving bed chromatography, hydrophobic interaction chromatography, gel filtration, ligand exchange chromatography, column chromatography, cation exchange adsorbent resin, and use of cation exchange resin.
19. Process according to any one of embodiment 1 to 18, wherein the step iii) of concentrating comprises one or more of nanofiltration, diafiltration, reverse osmosis, evaporation, wiped film evaporation, and falling film evaporation.
20. Process according to any one of embodiment 1 to 19, wherein the oligosaccharide solution comprises at least one oligosaccharide chosen from the list comprising fucosylated oligosaccharide, sialylated oligosaccharide, Lewis type antigen, N-acetylglucosamine containing neutral oligosaccharide, N-acetyllactosamine containing oligosaccharide, lacto-N-biose containing oligosaccharide, non-fucosylated neutral oligosaccharide, chitosan, chitosan oligosaccharide, heparosan, chondroitin sulphate, glycosaminoglycan oligosaccharide, heparin, heparan sulphate, chondroitin sulphate, dermatan sulphate, hyaluronan or hyaluronic acid and/or keratan sulphate.
21. Process according to any one of embodiment 1 to 20 wherein the oligosaccharide solution comprises a mammalian milk oligosaccharide (MMO), preferably a human milk oligosaccharide (HMO).
22. Process according to any one of embodiment 1 to 21, wherein the oligosaccharide solution comprises a neutral HMO, preferably selected from the group comprising 2′-fucosyl lactose, 3-fucosyl lactose, 2′,3-difucosyllactose, lacto-N-triose II, lacto-N-tetraose, lacto-N-neotetraose, lacto-N-fucopentaose I, lacto-N neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.
23. Process according to any one of embodiment 1 to 22, wherein the step i) comprises a first step of clarification by microfiltration.
24. Process according to any one of embodiment 1 to 22, wherein the step i) comprises a first step of clarification by centrifugation.
25. Process according to any one of embodiment 1 to 22, wherein the step i) comprises a first step of clarification by flocculation.
26. Process according to any one of embodiment 1 to 22, wherein the step i) comprises a first step of clarification by ultrafiltration.
27. Process according to any one of embodiment 1 to 26, wherein the step i) comprises ultrafiltration.
28. Process according to any one of embodiment 26 or 27, wherein in step i) the ultrafiltration has a molecular weight cut-off equal to or higher than 1 kDa, 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa.
29. Process according to any one of embodiment 1 to 28, wherein step i) comprises two consecutive ultrafiltrations, and wherein the membrane molecular weight cut-off of the first ultrafiltration is higher than that of the second ultrafiltration.
30. Process according to any one of embodiment 1 to 29, wherein step ii) comprises nanofiltration and/or electrodialysis.
31. Process according to embodiment 30, wherein the nanofiltration and/or electrodialysis is performed twice.
32. Process according to embodiment 31, wherein the nanofiltration and/or electrodialysis steps are performed consecutively.
33. Process according to any one of embodiment 26 to 32, wherein the ultrafiltration permeate of step i) is nanofiltered and/or electrodialysed in step ii).
34. Process according to any one of embodiment 1 to 22, 26 to 33 wherein the step i) is ultrafiltration, the step ii) is nanofiltration and/or electrodialysis treatment combined with a cation exchange treatment.
35. Process according to embodiment to 34, wherein the cation exchange treatment is preceded by ultrafiltration followed by nanofiltration and/or electrodialysis.
36. Process according to any one of embodiment 30 to 35, wherein the molecular weight cut-off of the nanofiltration membrane in step ii) is lower than that of the ultrafiltration membrane in step i).
37. Process according to any one of embodiment 1 to 36, wherein the cationic ion exchanger treatment is a strongly acidic cation exchanger treatment, preferably treatment with a strong cation exchange resin in H+ form, K+ or Na+ form.
38. Process according to any one of embodiment 1 to 37, wherein the pH of the eluent of the cation exchange treatment is controlled to keep the pH between 4 and 7, preferably by means of phosphoric acid, sulphuric acid, acetic acid, lactic acid, citric acid tartric acid, malic acid, succinic acid, fumaric acid.
39. Process according to any one of embodiment 1 to 38 that does not comprise electrodialysis.
40. Process according to any one of embodiment 1 to 38, wherein step ii) comprises electrodialysis.
41. Process according to one of the embodiments 1 to 40, wherein at least one of the purification steps i) to iii) is repeated at least one time during the process.
42. Process according to one of the embodiment 1 to 41, wherein after at least one of the purification steps i) or ii); the oligosaccharide solution is diafiltered and/or concentrated, preferably with a nanofiltration membrane, more preferably with a nanofiltration membrane having a size exclusion limit of less than or equal to 20 A, wherein most preferably the solution is diafiltered until a conductivity of less than or equal to 15 mS/cm, preferably less than or equal to 10 mS/cm, more preferably less than or equal to 5 mS/cm, is reached.
43. Process according to any one of embodiment 1 to 42, wherein the purified oligosaccharide solution has a Brix value of from about 8 to about 75%, preferably the purified oligosaccharide solution has a Brix value of from about 30 to about 65%.
44. Process according to any one of embodiment 1 to 43, wherein the purified oligosaccharide solution is sterile filtered and/or subjected to endotoxin removal, preferably by filtration of the purified oligosaccharide solution through a 3 kDa filter.
45. Process according to any one of embodiment 1 to 44, wherein step i) is preceded by an enzymatic treatment.
46. Process according to embodiment 45, wherein the enzymatic treatment comprises incubation of the broth with one or more enzymes selected from the group comprising: glycosidase, lactase, b-galactosidase, fucosidase, sialidase, maltase, amylase, hexaminidase, glucuronidase, trehalase, and invertase.
47. Process according to any one of embodiment 45 or 46, wherein the enzymatic treatment converts lactose and/or sucrose to monosaccharides.
48. Process of any one of embodiment 1 to 47, wherein the method further comprises decolorization.
49. Process according to any one of embodiment 1 to 48, wherein the purified oligosaccharide solution has an ash content below 10% (on total dry solid), preferably with Lead content lower than 0.1 mg/kg dry solid, Arsenic content lower than 0.2 mg/kg dry solid, Cadmium content lower than 0.1 mg/kg dry solid and/or Mercury content lower than 0.5 mg/kg dry solid.
50. Process according to any one of embodiment 1 to 49, wherein the purified oligosaccharide solution has a protein content below 100 mg per kg dry solid, DNA content below 10 ng per gram dry solid and/or endotoxin content below 10000 EU per gram dry solid, preferably the purified oligosaccharide solution is free of DNA, proteins, and/or recombinant genetic material.
51. Process according to any one of embodiment 1 to 50, wherein the cell cultivation or microbial fermentation comprises at least one cell, wherein the at least one cell is a cell of a bacterium, a fungus, a yeast, a plant, animal, or protozoan cell, preferably the cell is a cell of a micro-organism, wherein the micro-organism is a bacterium, preferably an Escherichia coli strain, more preferably an Escherichia coli strain that is a K-12 strain, even more preferably the Escherichia coli K-12 strain is E. coli MG1655.
52. Process according to any one of embodiment 1 to 50, wherein the cell cultivation or microbial fermentation comprises at least one cell, wherein the at least one cell is a yeast cell.
53. Process of embodiment 52, wherein the yeast is selected from the group comprising: Saccharomyces, Candida, Hansenula, Kluyveromyces, Pichia, Schizosaccharomyces, Schwanniomyces, Torulaspora, Yarrowia, and Zygosaccharomyces; preferably selected from the group comprising: Saccharomyces cerevisiae, Hansenula polymorpha, Kluyveromyces lactis, Kluyveromyces marxianus, Pichia pastoris, Pichia methanolica, Pichia stipites, Candida boidinii, Schizosaccharomyces pombe, Schwanniomyces occidentalis, Torulaspora delbrueckii, Yarrowia lipolytica, Zygosaccharomyces rouxii, and Zygosaccharomyces bailii.
54. Process according to any one of embodiment 1 to 53, wherein the cell cultivation or microbial fermentation comprises at least one cell, wherein the at least one cell is the cell of a micro-organism, wherein the micro-organism is an E. coli or yeast of lactose permease positive phenotype wherein the lactose permease is coded by the gene LacY or LAC12, respectively.
55. Process according to any one of embodiment 1 to 54, wherein the purified oligosaccharide solution is further concentrated to a syrup of at least 40% dry matter, the purified oligosaccharide solution is crystalized or the purified oligosaccharide solution is dried to a powder.
56. Process according to any one of embodiment 1 to 55, wherein step iii) comprises using vacuum evaporation or reverse osmosis or nanofiltration a) to an oligosaccharide concentration of >100 g/L, preferably >200 g/L, more preferably >300 g/L, more preferably >400 g/L, more preferably >500 g/L, more preferably >600 g/L, most preferably between 300 g/L and 650 g/L; and/or b) at a temperature of <60° C., preferably <50° C., more preferably 20° C. to 50° C., even more preferably 30° C. to 45° C., during vacuum evaporation or reverse osmosis; and/or c) at a temperature of <80° C., preferably <50° C., more preferably 20° C. to 50° C.
57. Process according to any one of embodiment 1 to 55, wherein the purified oligosaccharide solution comprises one oligosaccharide and is concentrated to a concentration of >1.5 M and cooled to a temperature <25° C., more preferable <8° C., to obtain crystalline material of the oligosaccharide.
58. Process according to any one of embodiment 1 to 55, wherein the purified oligosaccharide solution is dried.
59. Process according to embodiment 58, wherein the step of drying comprises any one or more of spray drying, lyophilization, evaporation, precipitation, spray freeze drying, freeze spray drying, band drying, belt drying, vacuum band drying, vacuum belt drying, drum drying, vacuum drum drying, roller drying, vacuum roller drying and other types of drying.
60. Process according to embodiment 59, wherein the purified oligosaccharide solution is spray-dried.
61. Process according to embodiment 59, wherein the drying is spray-drying or freeze-drying the purified oligosaccharide solution and preferably wherein the pH of the solution is lower than 5.0.
62. Process according to any one of embodiment 58 to 61, characterized in that the purified solution is spray-dried, particularly spray-dried at an oligosaccharide solution concentration of 20-60 (w/v), preferably 30-50 (w/v), more preferably 35-45 (w/v), a nozzle temperature of 110-150° C., preferably 120-140° C., more preferably 125-135° C. and/or an outlet temperature of 60-80° C., preferably 65-70° C.
63. The purified oligosaccharide solution, oligosaccharide or oligosaccharide mixture obtained according to the process according to any of embodiment 1 to 62.
64. Oligosaccharide producible with the process according to any one of the embodiment 1 to 62, wherein the oligosaccharide solution is preferably spray-dried, lyophilized or crystallized.
65. Oligosaccharide mixture producible with the process according to any one of embodiment 1 to 62, wherein the oligosaccharide solution is preferably spray-dried, lyophilized or concentrated to a syrup of at least 40% dry matter.
66. Oligosaccharide solution produced through cultivation or fermentation wherein the oligosaccharide solution is purified without anion exchange and wherein the oligosaccharide solution contains less than 10% ash.
67. Spray dried oligosaccharide or oligosaccharide mixture produced through cultivation or fermentation wherein the oligosaccharide or oligosaccharide mixture is purified without anion exchange and wherein the spray dried oligosaccharide or oligosaccharide mixture contains less than 10% ash.
68. Dried powder obtained according to any one of the process of embodiment 58 to 62, wherein the dried powder contains ≤15%-wt. of water, preferably ≤10%-wt. of water, more preferably ≤7%-wt. of water, most preferably ≤5%-wt. of water.
69. Spray dried powder obtained according to any one of the process of embodiment 58 to 62, wherein the powder has a mean particle size of 50 to 250 μm, determined by laser diffraction; preferably the powder has a mean particle size of 95 to 120 μm, more preferably the powder has a mean particle size of 110 to 120 μm.
70. Dried powder obtainable according to the process according to any one of embodiment 58 to 62, wherein the powder exhibits:
   a loose bulk density of from about 500 to 700 g/L,
   a 100× tapped bulk density of from about 600 to about 850 g/L,
   a 625× tapped bulk density of from about 600 to about 900 g/L, and/or
   a 1250× tapped bulk density of from about 650 to about 900 g/L.
71. Dried powder obtained according to 70, wherein the powder exhibits:
   a loose bulk density of from about 600 to 700 g/L,
   a 100× tapped bulk density of from about 750 to about 850 g/L,
   a 625× tapped bulk density of from about 750 to about 850 g/L, and/or
   a 1250× tapped bulk density of from about 850 to about 900 g/L.
72. Dried powder obtained according to 70, wherein the powder exhibits:
   a loose bulk density of from about 500 to 600 g/L,
   a 100× tapped bulk density of from about 600 to about 700 g/L,
   a 625× tapped bulk density of from about 700 to about 800 g/L, and/or
   a 1250× tapped bulk density of from about 750 to about 800 g/L.
73. Dried powder according to any one of embodiment 64, 65, 67 to 72 wherein the powder when redissolved in water at a concentration of 10% (mass on volume) provides a solution with a pH between 4 and 7, preferably with a pH between 4 and 6.
74. Oligosaccharide according to any one of embodiment 63, 64 or 67 to 73, wherein the oligosaccharide is a mammalian milk oligosaccharide, preferably a human milk oligosaccharide (HMO).
75. Oligosaccharide mixture according to any one of embodiment 64, 65 or 67 to 73, wherein the mixture comprises a mammalian milk oligosaccharide, preferably a human milk oligosaccharide (HMO).
76. HMO according to any one of embodiment 74 or 75, wherein the HMO is a neutral HMO selected from the group comprising 2′-fucosyl lactose, 3-fucosyl lactose, 2′,3-difucosyllactose, lacto-N-triose II, lacto-N-tetraose, lacto-N-neotetraose, lacto-N-fucopentaose I, lacto-N neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.
77. Oligosaccharide or oligosaccharide mixture according to any one of embodiments 63 to 70, wherein the oligosaccharide or oligosaccharide mixture a) has a conductivity of less than 1 mS/cm at a 300 g/l solution; b) is free of recombinant DNA material, optionally free of any DNA; and/or c) is free of proteins derived from the recombinant micro-organism, optionally free of any proteins.
78. Oligosaccharide or oligosaccharide mixture according to any one of embodiment 63 to 77 for use in medicine, preferably for use in prophylaxis or therapy of a gastrointestinal disorder.
79. Use of the purified of oligosaccharides obtained according to the process of any one of embodiment 1 to 62 in a food or feed preparation, in a dietary supplement, in a cosmetic ingredient or in a pharmaceutical ingredient.
80. Use of the purified oligosaccharide solution according to any one of embodiment 63 to 77 in a food or feed preparation, in a dietary supplement, in a cosmetic ingredient or in a pharmaceutical ingredient.
81. Use according to any one of embodiment 79 or 80, wherein the food is a human food, preferably infant food and/or an infant formula or an infant supplement.
82. Use of an HMO according to any one of embodiment 74 to 76 as additive in food, preferably as additive in human food and/or pet food, more preferably as additive in human baby food.
83. Use according to any one of embodiment 79 or 80 wherein the feed is a pet food, animal milk replacer, veterinary product, post weaning feed, or creep feed.

This disclosure will be described in more detail in the examples.

### EXAMPLES

### Example 1. Materials and Methods

Media and Cultivation

The Luria Broth (LB) medium comprised 1% tryptone peptone (Difco, Erembodegem, Belgium), 0.5% yeast extract (Difco) and 0.5% sodium chloride (VWR. Leuven, Belgium). The minimal medium used in the cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄·7H₂O, 30 g/L sucrose or 30 g/L glycerol, 1 ml/L vitamin solution, 100 μl/L molybdate solution, and 1 mL/L selenium solution. As specified in the respective examples, 0.30 g/L sialic acid, 20 g/L lactose, 20 g/L LacNAc, 20 g/L LNnT, 20 g/L LNT and/or 20 g/L LNB were additionally added to the medium as precursor(s). The minimal medium was set to a pH of 7 with 1M KOH. Vitamin solution comprised 3.6 g/L FeCl₂·4H₂O, 5 g/L CaCl₂·2H₂O, 1.3 g/L MnCl₂·2H₂O, 0.38 g/L CuCl₂·2H₂O, 0.5 g/L CoCl₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L SeO₂.

The minimal medium for fermentations contained 6.75 g/L NH₄Cl, 1.25 g/L (NH₄)2SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 20 g/L lactose, 20 g/L LacNAc, 20 g/L LNnT, 20 g/L LNT and/or 20 g/L LNB were additionally added to the medium as precursor(s).

Complex medium was sterilized by autoclaving (121° C., 21 min) and minimal medium by filtration (0.22 μm Sartorius).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 or 30L L bioreactor was then inoculated (250 mL inoculum in 2 L batch medium or 1L in 17L batch medium); the process was controlled by MFCS control software (Sartorius Stedim Biotech, Melsungen, Germany). Culturing conditions were set to 37° C., and maximal stirring; pressure gas flow rates were dependent on the strain and bioreactor. The pH was controlled at 6.8 using 0.5 M H₂S0₄ and 20% NH₄OH. The exhaust gas was cooled. 10% solution of silicone antifoaming agent was added when foaming raised during the fermentation.

Strains and Mutations

Escherichia coli K12 MG1655 [λ-, F-, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an OD600 nm of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, re-purified from an agarose gel, and suspended in elution buffer (5 mM Tris, pH 8.0). Selected mutants were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock ins are checked with control primers.

In one example for GDP-fucose and fucosylated oligosaccharide production, the mutant strain was derived from E. coli K12 MG1655 comprising knock-outs of the E. coli wcaJ and thyA genes and genomic knock-ins of constitutive expression constructs containing a sucrose transporter like e.g., CscB originating from E. coli W (UniProt ID E0IXR1), a fructose kinase like e.g., frk originating from Zymomonas mobilis (ZmFrk) (UniProt ID Q03417), a sucrose phosphorylase like e.g., BaSP originating from Bifidobacterium adolescentis (UniProt ID A0ZZH6), additionally comprising expression plasmids with constitutive expression constructs for an alpha-1,2-fucosyltransferase like e.g., HpFutC from H. pylori (GenBank No. AAD29863.1) and/or an alpha-1,3-fucosyltransferase like e.g., HpFucT from H. pylori (UniProt ID O30511) and with a constitutive expression construct for the E. coli thyA (UniProt ID P0A884) as selective marker. The constitutive expression constructs of the fucosyltransferase genes can also be present in the mutant E. coli strain via genomic knock-ins. GDP-fucose production can further be optimized in the mutant E. coli strain by genomic knock-outs of the E. coli genes comprising glgC, agp, pfkA, pfkB, pgi, arcA, iclR, pgi and lon as described in WO 2016075243 and WO 2012007481. GDP-fucose production can additionally be optimized comprising genomic knock-ins of constitutive expression constructs for a mannose-6-phosphate isomerases like e.g., manA from E. coli (UniProt ID P00946), a phosphomannomutase like e.g., manB from E. coli (UniProt ID P24175), a mannose-1-phosphate guanylyltransferase like e.g., manC from E. coli (UniProt ID P24174), a GDP-mannose 4,6-dehydratase like e.g., gmd from E. coli (UniProt ID P0AC88) and a GDP-L-fucose synthase like e.g., fcl from E. coli (UniProt ID P32055). GDP-fucose production can also be obtained by genomic knock-outs of the E. coli fucK and fucI genes together with genomic knock-ins of constitutive expression constructs containing fucose permease like e.g., fucP from E. coli (UniProt ID P11551) and a bifunctional enzyme with fucose kinase/fucose-1-phosphate guanylyltransferase activity like e.g., fkp from Bacteroides fragilis (UniProt ID SUV40286.1). If the mutant strain producing GDP-fucose was intended to make fucosylated lactose structures, the strain was additionally modified with genomic knock-outs of the E. coli LacZ, LacY and LacA genes and with a genomic knock-in of a constitutive expression construct for a lactose permease like e.g., the E. coli LacY (UniProt ID P02920).

In an example to produce lacto-N-triose (LN3, GlcNAc-b1,3-Gal-b1,4-Glc), the mutant strain was derived from E. coli K12 MG1655 and modified with a knock-out of the E. coli lacZ, lacY, lacA and nagB genes and with genomic knock-ins of constitutive transcriptional units for a lactose permease like e.g., the E. coli LacY (UniProt ID P02920) and a galactoside beta-1,3-N-acetylglucosaminyltransferase like e.g., lgtA (UniProt ID Q9JXQ6) from N. meningitidis.

In an example for production of LN3 derived oligosaccharides like lacto-N-tetraose (LNT, Gal-b1,3-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,3-galactosyltransferase like e.g., wbgO from E. coli O55:H7.

In an example for production of LN3 derived oligosaccharides like lacto-N-neotetraose (LNnT, Gal-b1,4-GlcNAc-b1,3-Gal-b1,4-Glc), the mutant LN3 producing strain was further modified with a constitutive transcriptional unit delivered to the strain either via genomic knock-in or from an expression plasmid for an N-acetylglucosamine beta-1,4-galactosyltransferase like e.g., lgtB from Neisseria meningitidis.

Optical Density

Cell density of the cultures was frequently monitored by measuring optical density at 600 nm (Implen Nanophotometer NP80, Westburg, Belgium or with a Spark 10M microplate reader, Tecan, Switzerland).

Analytical Analysis

Standards such as but not limited to sucrose, lactose, N-acetyllactosamine (LacNAc, Gal-b1,4-GlcNAc), lacto-N-biose (LNB, Gal-b1,3-GlcNAc), fucosylated LacNAc (2′FLacNAc, 3-FLacNAc), sialylated LacNAc, (3′ SLacNAc, 6′ SLacNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1 ×100 mm; 130 Å; 1.7 μm) column with an Acquity UPLC BEH Amide VanGuard column, 130 Å, 2.1×5 mm. The column temperature was 50° C. The mobile phase comprised a ¼ water and ¾ acetonitrile solution to which 0.2% triethylamine was added. The method was isocratic with a flow of 0.130 mL/min. The ELS detector had a drift tube temperature of 50° C. and the N2 gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was ° C. The mobile phase comprised a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase comprised a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionization (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

For identification of the single oligosaccharides in the mixture of oligosaccharides produced as described herein, the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the saccharide methods such as, e.g., acid-catalyzed hydrolysis, HPLC (high performance liquid chromatography) or GLC (gas-liquid chromatography) (after conversion to alditol acetates) may be used. To determine the glycosidic linkages, the saccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the oligosaccharide sequence, a partial depolymerization is carried out using an acid or enzymes to determine the structures. To identify the anomeric configuration, the oligosaccharide is subjected to enzymatic analysis, e.g., it is contacted with an enzyme that is specific for a particular type of linkage, e.g., beta-galactosidase, or alpha-glucosidase, etc., and NMR may be used to analyze the products.

Ash Content

The ash content is a measure of the total amount of minerals present within a food or ingredients such as oligosaccharides, whereas the mineral content is a measure of the amount of specific inorganic components present within a food, such as Ca, Na, K, Mg, phosphate, sulphate and Cl. Determination of the ash and mineral content of foods or oligosaccharides is important for a number of reasons: Nutritional labeling. The concentration and type of minerals present must often be stipulated on the label of a food or ingredient such as oligosaccharides. The quality of many foods depends on the concentration and type of minerals they contain, including their taste, appearance, texture and stability. Microbiological stability. High mineral contents are sometimes used to retard the growth of certain microorganisms. Nutrition. Some minerals are essential to a healthy diet (e.g., calcium, phosphorous, potassium and sodium) whereas others can be toxic (e.g., lead, mercury, cadmium and aluminum). Processing. It is often important to know the mineral content of foods/products during processing because this affects the physicochemical properties of foods or ingredient such as oligosaccharides.

Ash is the inorganic residue remaining after the water and organic matter have been removed by heating in the presence of oxidizing agents, which provides a measure of the total amount of minerals within a food. Analytical techniques for providing information about the total mineral content are based on the fact that the minerals (the analyte) can be distinguished from all the other components (the matrix) within a food or ingredient in some measurable way. The most widely used methods are based on the fact that minerals are not destroyed by heating, and that they have a low volatility compared to other food components. The three main types of analytical procedure used to determine the ash content of foods are based on this principle: dry ashing, wet ashing and low temperature plasma dry ashing. The method chosen for a particular analysis depends on the reason for carrying out the analysis, the type of food or ingredient analyzed and the equipment available. Ashing may also be used as the first step in preparing samples for analysis of specific minerals, by atomic spectroscopy or the various traditional methods described below.

For the sample preparation a sample whose composition represents that of the ingredient is selected to ensure that its composition does not change significantly prior to analysis. For instance, a dry oligosaccharide sample is generally hygroscopic and the selected sample should be kept under dry conditions avoiding the absorption of water. Typically, samples of 1-10 g are used in the analysis of ash content. Solid ingredients are finely ground and then carefully mixed to facilitate the choice of a representative sample. Before carrying out an ash analysis, samples that are high in moisture or in solution are generally dried to prevent spattering during ashing. Other possible problems include contamination of samples by minerals in grinders, glassware or crucibles that come into contact with the sample during the analysis. For the same reason, deionized water is used when preparing samples and the same is used in the blank sample.

Dry ashing procedures use a high temperature muffle furnace capable of maintaining temperatures of between 500 and 600° C. Water and other volatile materials are vaporized and organic substances are burned in the presence of the oxygen in air to CO₂, H₂O and N₂. Most minerals are converted to oxides, sulfates, phosphates, chlorides or silicates. Although most minerals have fairly low volatility at these high temperatures, some are volatile and may be partially lost, e.g., iron, lead and mercury, for these minerals ICP-MS analysis of the product is more appropriate for quantification.

The food sample is weighed before and after ashing to determine the concentration of ash present. The ash content can be expressed on dry basis is calculated by dividing the mass of the ashed material, ingredient, or food by the mass of the dry material, ingredient, or food before ashing. Multiplied with 100, this gives the percentage of ash in the material, ingredient, or food. In a similar way the wet ash percentage can be determined for liquid products, wherein the mass of the liquid before and after ashing is used instead of the mass of the dry material, ingredient, or food.

Heavy Metal Determination

A robust general inductively coupled plasma-mass spectrometry (ICP-MS) based method was used for the detection and quantitation for each of the following elements: arsenic (As), selenium (Se), cadmium (Cd), tin (Sn), lead (Pb), silver (Ag), palladium (Pd), platinum (Pt), mercury (Hg), molybdenum (Mo), sodium (Na), potassium (K), Calcium (Ca), Magnesium (Mg), Iron (Fe), zinc (Zn), manganese (Mn), Phosphorus (P), selenium (Se).

Nitric acid (≥65%, Sigma-Aldrich) was used for microwave digestion and standard/sample preparation. All dilutions were done using 18.2 MΩ·cm (Millipore, Bedford, MA, USA) de-ionized water (DIW). About 0.2 g of each oligosaccharide, ingredient, sample were digested in 5 mL of HNO3 using the microwave digestion (CEM, Mars 6) program 15 minutes (min) ramping time and 15 min holding time at 100 W and 50° C. followed by 15 min ramping time and 20 min holding time at 1800 W and 210° C. The samples were cooled after digestion for 30 minutes. 1. The fully digested samples were then diluted to 50 mL with DIW.

Analyses were carried out using a standard Agilent 7800 ICP-MS, which includes the fourth-generation ORS cell system for effective control of polyatomic interferences using helium collision mode (He mode). The ORS controls polyatomic interferences using He to reduce the transmission of all common matrix-based polyatomic interferences. Smaller, faster analyte ions are separated from larger, slower interference-ions using kinetic energy discrimination (KED). All elements, except Se, were measured in He mode with a flow rate of 5 mL/min. Se was measured in High Energy He (HEHe) mode, using a cell gas flow rate of 10 mL/min. The 7800 ICP-MS was configured with the standard sample introduction system consisting of a MicroMist glass concentric nebulizer, quartz spray chamber, quartz torch with 2.5 mm i.d. injector, and nickel interface cones. The ICP-MS operating conditions are: 1550 W RF power, 8 mm sampling depth, 1.16 l/min nebulizing gas, autotuned lens tuning, 5 or 10 ml/min helium gas flow, 5 V KED.

Dry Matter and Moisture Content Quantification

Sartorius MA150 Infrared Moisture Analyzer is used to determine the dry matter content of the oligosaccharides. 0.5 g of oligosaccharide is weighed on an analytical balance and is dried in the infrared moisture analyzer until the weight of the sample is stable. The mass of the dried sample divided by the mass of the sample before drying gives the dry matter content (in percent) of the oligosaccharides or sample including oligosaccharides. In a similar way a liquid sample is weighed, however, the amount of liquid weighed is adapted to the expected amount of dry matter in the liquid, so the mass of the dry matter is properly measurable on an analytical balance.

A moisture analyzer measures the dry matter, but not the water content. Karl Fisher titration is used to determine the amount of water present in a powder, ingredient of food. The KF titration is carried out with a Karl Fischer titrator DL31 from Mettler Toledo using the two-component technique with Hydra-Point Solvent G and Hydra-Point titrant (5 mg H₂O/ml), both purchased from J. T. Baker (Deventer, Holland). The polarizing current for bipotentiometric end-point determination was 20 microA and the stop voltage 100 mV. The end-point criterion was the drift stabilization (15 micro gram H₂O min⁻¹) or maximum titration time (10 min).

The moisture content (MC) of sample was calculated using the following equation:

MC=V_KF_W_eq 100/W_sample; where V_KF is the consumption of titrant in mL, W_eq the titer of titrant in mg H₂O/mL and W_sample the weight of sample in mg.

Biomass Dry Mass Content (Cell Dry Mass)

Cell dry weight was obtained by centrifugation (15 min, 5000 g) of 20 g broth in pre-dried (70° C. overnight) and weighted falcons. The pellets were subsequently washed once with 20 ml physiological solution (9 g/l NaCl) and dried at 70° C. to a constant weight. The final weight was corrected for the added sodium chloride to the sample.

Protein Quantification

For protein quantification a method is used that is compatible with reducing agents, such as reducing sugars or oligosaccharides with a reducing end. To this end, a Bradford assay (Thermo Scientific, Pierce) was used with a linear range between 1 and 1500 μg/ml. The assay was calibrated with a standard curve of BSA. The protein content of dried oligosaccharide products was quantified by dissolving a pre-weighed quantify in 18.2 MΩ·cm (Millipore, Bedford, MA, USA) de-ionized water (DIW) up to a quantity of 50% (m/v). The amount of protein is measured at 595 nm and converted to concentration with the calibration curve based on BSA.

DNA Quantification

Production host specific DNA residue is quantified by RT-qPCR, for which specific primers on the host are designed so that residual DNA of the production host in amplified. The RT-qPCR was performed according to the standard protocol of a kit obtained from Sigma and was based on SYBR Green detection.

Total DNA is measured by means of a Threshold assay (Molecular Devices), based on an immunoassay allowing to measure as low as 2 pg of DNA in a sample in solution. Double stranded DNA is measured by means of SpectraMax® Quant™ AccuBlue™ Pico dsDNA Assay Kit (Molecular Devices) having a linear range between 5 pg and 3 ng of dsDNA.

Endotoxin Measurement

Endotoxin in the liquid was measured by means of a LAL test.

Laser Diffraction

The powder particle size can be assessed by laser diffraction. The system detects scattered and diffracted light by an array of concentrically arranged sensor elements. The software-algorithm is then approximating the particle counts by calculating the z-values of the light intensity values, which arrive at the different sensor elements. The analysis can be executed using a SALD-7500 Aggregate Sizer (Shimadzu Corporation, Kyoto, Japan) quantitative laser diffraction system (qLD).

A small amount (spatula tip) of each sample can be dispersed in 2 ml isooctane and homogenized by ultrasonication for five minutes. The dispersion will then be transferred into a batch cell filled with isooctane and analyzed in manual mode.

Data acquisition settings can be as follows: Signal Averaging Count per Measurement: 128, Signal Accumulation Count: 3, and Interval: 2 seconds.

Prior to measurement, the system can be blanked with isooctane. Each sample dispersion will be measured 3 times and the mean values and the standard deviation will be reported. Data can be evaluated using software WING SALD II version V3.1. When the refractive index of the sample is unknown, the refractive index of sugar (disaccharide) particles (1.530) can be used for determination of size distribution profiles. Size values for mean and median diameter are reported. The mean particle sizes for all samples are very similar due to the spray dryer settings used. In addition, the particle size distribution will show the presence of one main size population for all of the samples.

### Example 2. Production of an Oligosaccharide Mixture Comprising 2′FL, 3-FL and DiFL with a Modified E. coli Host in Fed-Batch Fermentations

An E. coli K12 strain modified for GDP-fucose production, as described in the art or as described in Example 1, was sequentially transformed with a first plasmid expressing a constitutive transcriptional unit for the H. pylori alpha-1,2-fucosyltransferase and a second compatible plasmid expressing a constitutive transcriptional unit for the H. pylori alpha-1,3-fucosyltransferase. This modified mutant strain was evaluated in a batch and in a fed-batch fermentation process. Fed-batch fermentations at bioreactor scale (5 and 30L) were performed as described in Example 1. In these examples, sucrose was used as a carbon source and lactose was added in the batch medium as a precursor. Regular broth samples were taken and the production of 2′FL, 3-FL and DiFL was measured using UPLC as described in Example 1. The experiment demonstrated that broth samples taken at the end of batch phase comprised an oligosaccharide mixture of 2′FL and 3-FL together with unmodified lactose, whereas broth samples taken at the end of the fed-batch phase comprised an oligosaccharide mixture of 2′FL, 3-FL and DiFL. As the ratios of lactose, 2′FL, 3-FL and DiFL changed over time during fed-batch, they could be manipulated during the fermentation process by discontinuation of the fermentation process at a desired time in fed-batch phase.

The resulting broth is clarified as described in Example 14. In a separate example the cells were lysed to increase the release of oligosaccharides as described in Example 13 and further clarified as described in Example 14.

### Example 3. Production of an Oligosaccharide Mixture Comprising LN3, Sialylated LN3, LNT, LSTa and 3′SL in Fermentation Broth of Mutant E. coli Strains when Evaluated in a Fed-Batch Fermentation Process with Glycerol as Carbon Source, Sialic Acid and Lactose as Precursors

An E. coli strain modified to produce LNT, as described in the art or as described in Example 1, was further modified with a genomic knock-out of the E. coli lacZ gene and transformed with an expression plasmid containing constitutive expression cassettes for the NeuA gene from P. multocida coding for N-acylneuraminate cytidylyltransferase and the α-2,3-sialyltransferase gene from P. multocida. This strain produces a mixture of oligosaccharides comprising LN3, 3′-sialylated LN3 (Neu5Ac-a2,3-GlcNAc-b1,3-Gal-b1,4-Glc), LNT, 3′SL and LSTa (Neu5Ac-a2,3-Gal-b1,3-GlcNAc-b1,3-Gal-b1,4-Glc) and was grown in a batch and in a fed-batch fermentation process in a 5L and 30L bioreactor. Fed-batch fermentations at bioreactor scale were performed as described in Example 1. In these examples, glycerol was used as a carbon source and lactose was added in the batch medium as precursor. During fed-batch, also sialic acid was added via an additional feed. Regular broth samples were taken, and sugars produced were measured as described in Example 1. UPLC analysis shows that fermentation broth of the selected strain taken after the batch phase contains lactose, LN3 and LNT, whereas fermentation broth of the selected strain taken after the fed-batch phase comprises an oligosaccharide mixture comprising LN3, 3′-sialylated LN3 (Neu5Ac-a2,3-GlcNAc-b1,3-Gal-b1,4-Glc), LNT, LSTa and 3′SL.

The resulting broth is clarified as described in Example 14. In a separate example the cells were lysed to increase the release of oligosaccharides as described in Example 13 and further clarified as described in Example 14.

### Example 4. Production of an Oligosaccharide Mixture Comprising LN3, Sialylated LN3, LNT, LSTa and 3′SL in Fermentation Broth of Mutant E. coli Strains when Evaluated in a Fed-Batch Fermentation Process with Sucrose and Lactose

An E. coli strain modified to produce sialic acid as described in WO 2018122225 was further modified with a genomic knock-in of constitutive transcriptional units for the galactoside beta-1,3-N-acetylglucosaminyltransferase gene (LgtA) from N. meningitidis and for the N-acetylglucosamine beta-1,3-galactosyltransferase gene (WbgO) from E. coli O55: to allow production of LNT. In a next step, the novel strain was further modified with a genomic knock-out of the E. coli lacZ gene and transformed with an expression plasmid having constitutive transcriptional units for the NeuA gene from P. multocida coding for N-acylneuraminate cytidylyltransferase and the α-2,3-sialyltransferase gene from P. multocida. The novel strain produces an oligosaccharide mixture comprising LN3, 3′-sialylated LN3 (Neu5Ac-a2,3-GlcNAc-b1,3-Gal-b1,4-Glc), LNT, 3′SL and LSTa when evaluated in a growth experiment according to the culture conditions provided in Example 1, in which the culture medium contains sucrose as carbon source and lactose as precursor.

This mutant was selected for further evaluation in a fed-batch fermentation process in a 5L and 30L bioreactor. Fed-batch fermentations at bioreactor scale were performed as described in Example 1. In these examples, sucrose was used as a carbon source and lactose was added in the batch medium as precursor. Regular broth samples are taken, and sugars produced are measured as described in Example 1. UPLC analysis shows that fermentation broth of the selected strain taken after the batch phase contains lactose, LN3, 3′SL, and LNT, whereas fermentation broth of the selected strain taken after the fed-batch phase comprises an oligosaccharide mixture comprising LN3, 3′-sialylated LN3 (Neu5Ac-a2,3-GlcNAc-b1,3-Gal-b1,4-Glc), LNT, LSTa and 3′SL.

The resulting broth is clarified as described in Example 14. In a separate example the cells were lysed to increase the release of oligosaccharides as described in Example 13 and further clarified as described in Example 14.

### Example 5. Production of an Oligosaccharide Mixture Comprising LN3, Sialylated LN3, LNnT, Para-Lacto-N-Neohexaose, Di-Sialylated LNnT, LSTc and 6′SL in Fermentation Broth of Mutant E. coli Strains when Evaluated in a Fed-Batch Fermentation Process with Glycerol, Sialic Acid and Lactose

An E. coli strain modified to produce LNnT, as described in the art or as described in Example 1, was further modified with a genomic knock-out of the E. coli lacZ gene and transformed with an expression plasmid containing constitutive expression cassettes for the NeuA gene from P. multocida coding for N-acylneuraminate cytidylyltransferase and one selected α-2,6-sialyltransferase gene from P. damselae The strains produce a mixture of oligosaccharides comprising 6′SL, LN3, 6′-sialylated LN3 (Neu5Ac-a2,6-(GlcNAc-b1,3)-Gal-b1,4-Glc), LNnT and LSTc (Neu5Ac-a2,6-Gal-b1,4-GlcNAc-b1,3-Gal-b1,4-Glc).

This mutant strain was cultivated in a batch and fed-batch fermentation process in a 5L and 30L bioreactor. Fed-batch fermentations at bioreactor scale were performed as described in Example 1. In these examples, glycerol is used as a carbon source and lactose was added in the batch medium as precursor. During fed-batch, also sialic acid was added via an additional feed. Regular broth samples were taken, and sugars produced were measured as described in Example 1. UPLC analysis shows that fermentation broth of the selected strain taken after the batch phase contains lactose, LN3, and LNnT, whereas fermentation broth of the selected strain taken after the fed-batch phase comprises an oligosaccharide mixture comprising LN3, 6′-sialylated LN3 (Neu5Ac-a2,6-(GlcNAc-b1,3)-Gal-b1,4-Glc), LNnT, LSTc and 6′SL. At end of fed-batch, the mixture also comprises para-lacto-N-neohexaose (pLNnH) and di-sialylated LNnT, two structures that were not detected in growth experiment assays due to limited detection levels and overall smaller production levels.

The resulting broth is clarified as described in Example 14. In a separate example the cells were lysed to increase the release of oligosaccharides as described in Example 13 and further clarified as described in Example 14.

### Example 6. Production of an Oligosaccharide Mixture Comprising LN3, Sialylated LN3, LNnT, Para-Lacto-N-Neohexaose, Di-Sialylated LNnT, LSTc and 6′SL in Fermentation Broth of Mutant E. coli Strains when Evaluated in a Fed-Batch Fermentation Process with Sucrose and Lactose

An E. coli strain modified to produce sialic acid as described in WO 2018122225 was further modified with a genomic knock-in of constitutive transcriptional units for the LgtA gene from N. meningitidis and for the LgtB gene from N. meningitidis to allow production of LNnT. In a next step, the novel strain was further modified with a genomic knock-out of the E. coli lacZ gene and transformed with an expression plasmid having constitutive transcriptional units for the NeuA gene from P. multocida coding for N-acylneuraminate cytidylyltransferase and the α-2,6-sialyltransferase gene from Photobacterium sp. JT-ISH-224. This strain produces a mixture of oligosaccharides comprising LN3, 6′-sialylated LN3 (Neu5Ac-a2,6-(GlcNAc-b1,3)-Gal-b1,4-Glc), 6′SL, LNnT and LSTc.

This strain was grown in a batch and fed-batch fermentation process in a 5L and bioreactor. Fed-batch fermentations at bioreactor scale were performed as described in Example 1. In these examples, sucrose was used as a carbon source and lactose was added in the batch medium as precursor. Regular broth samples were taken, and sugars produced were measured as described in Example 1. UPLC analysis shows that fermentation broth of the selected strain taken after the batch phase contains lactose, LN3, 6′SL, and LNnT, whereas fermentation broth of the selected strain taken after the fed-batch phase comprises an oligosaccharide mixture comprising LN3, 6′-sialylated LN3 (Neu5Ac-a-2,6-(GlcNAc-b-1,3)-Gal-b-1,4-Glc), LNnT, LSTc and 6′SL. At end of fed-batch, the mixture also comprises para-lacto-N-neohexaose and di-sialylated LNnT, two structures that were not detected in growth experiment assays due to limited detection levels and overall smaller production levels.

The resulting broth is clarified as described in Example 14. In a separate example the cells were lysed to increase the release of oligosaccharides as described in example 13 and further clarified as described in Example 14.

### Example 7. Production of an Oligosaccharide Mixture Comprising LNT, LNnT and Poly-Galactosylated Structures in a Modified E. coli Host when Evaluated in Fed-Batch Fermentations

The mutant strain for LNnT, as described in the art or as described in Example 1, is modified with constitutive transcriptional unit of N-acetylglucosamine beta-1,4-galactosyltransferase gene (LgtB) from N. meningitidis in one or more copies. To enhance UDP-galactose production the genes ushA and galT are knocked out. The mutant E. coli strains is further modified with a genomic knock-in of a constitutive transcriptional unit for the UDP-glucose-4-epimerase gene (galE) gene from E. coli, the phosphoglucosamine mutase (glmM) gene from E. coli and the N-acetylglucosamine-1-phosphate uridyltransferase/glucosamine-1-phosphate acetyltransferase (glmU) gene from E. coli. The mutant strain is further mutated for growth on sucrose via genomic knock-ins of constitutive transcriptional units containing a sucrose transporter (CscB) gene from E. coli W, a fructose kinase gene (Frk) originating and a sucrose phosphorylase originating from B. adolescentis. This strain is further modified with genomic knock-ins of constitutive transcriptional units for the WbgO gene from E. coli O55:H7.

The final mutant strain produces a mixture of Lacto-N-triose II (LN3), Lacto-N-neotetraose (LNnT), Lacto-N-tetraose (LNT), para-Lacto-N-neopentaose, para-Lacto-N-pentaose, para-Lacto-N-neohexaose, para-Lacto-N-hexaose, beta-(1,3)Galactosyl-para-Lacto-N-neopentaose and beta-(1,4)Galactosyl-para-Lacto-N-pentaose.

This mutant strain is evaluated in a batch and fed-batch fermentation process in a 5L and 30L bioreactor as described in Example 1. In this example sucrose is used as a carbon source and lactose is added in the batch medium as precursor. Regular broth samples are taken, and sugars produced are measured as described in Example 1. UPLC analysis shows that fermentation broth of the selected strain taken at regular timepoints in fed-batch phase contains an oligosaccharide mixture comprising Lacto-N-triose II (LN3), Lacto-N-neotetraose (LNnT), Lacto-N-tetraose (LNT), para-Lacto-N-neopentaose, para-Lacto-N-pentaose, para-Lacto-N-neohexaose, para-Lacto-N-hexaose, beta-(1,3)Galactosyl-para-Lacto-N-neopentaose and beta-(1,4)Galactosyl-para-Lacto-N-pentaose.

The resulting broth is clarified as described in Example 14. In a separate example the cells were lysed to increase the release of oligosaccharides as described in Example 13 and further clarified as described in Example 14.

### Example 8. Production of an Oligosaccharide Mixture Comprising 2′FL, 3-FL, DiFL, LN3, LNT and LNFP-I with a Modified E. coli Host

An E. coli strain modified for GDP-fucose as described in the examples above was further modified to express the glmS*54 gene from E. coli, the LgtA gene from N. meningitidis, the WbgO gene from E. coli O55:H7, the a-1,2-fucosyltransferase gene from H. pylori, and the a-1,3-fucosyltransferase gene (HpFucT). The novel strain produces an oligosaccharide mixture comprising 2′FL, 3-FL, DiFL, LN3, LNT and LNFP-I (Fuc-a1,2-Gal-b1,3-GlcNAc-b1,3-Gal-b1,4-Glc) in a growth experiment according to the culture conditions provided in Example 1, in which the culture medium contains sucrose as carbon source and lactose as precursor. This mutant strain is evaluated in a batch and fed-batch fermentation process in a 5L and 30L bioreactor as described in Example 1. In this example sucrose is used as a carbon source and lactose is added in the batch medium as precursor. Regular broth samples are taken, and sugars produced are measured as described in Example 1. UPLC analysis shows that fermentation broth of the selected strain taken at regular timepoints in fed-batch phase contains an oligosaccharide mixture comprising 2′FL, 3-FL, DiFL, LN3, LNT and LNFP-I (Fuc-a1,2-Gal-b1,3-GlcNAc-b1,3-Gal-b1,4-Glc).

The resulting broth is clarified as described in Example 14. In a separate example the cells were lysed to increase the release of oligosaccharides as described in Example 13 and further clarified as described in Example 14.

### Example 9. Production of an Oligosaccharide Mixture Comprising Fucosylated and Sialylated Oligosaccharide Structures with a Modified E. coli Host

An E. coli strain adapted for sialic acid production as described in WO 2018122225 was further modified with a genomic knock-out of the E. coli weal gene to increase the intracellular pool of GDP-fucose and genomic knock-ins of constitutive expression cassettes for the LgtA gene from N. meningitidis and the WbgO gene from E. coli O55:H7. In a next step, the novel strain was transformed with two compatible expression plasmids wherein a first plasmid pMF_2 contained (a) constitutive expression unit(s) for two fucosyltransferase genes, H. pylori alpha-1,2-fucosyltransferase gene (HpFutC) and the H. pylori alpha-1,3-fucosyltransferase gene (HpFucT), and wherein a second plasmid pMS_2 contained constitutive expression units for two sialyltransferase genes, alpha-2,3-sialyltransferase from P. multocida and alpha-2,6-sialyltransferase (PdST6) from Photobacterium damselae, and the NeuA gene from P. multocida coding for N-acylneuraminate cytidylyltransferase. This strain produces an oligosaccharide mixture comprising fucosylated and sialylated lactose, LNB, fucosylated and sialylated LNB, LN3, sialylated LN3, LNT and fucosylated and sialylated LNT structures in whole broth samples. The strain was grown in an experiment according to the culture conditions provided in Example 1 in which the cultivation contains sucrose as carbon source and lactose as precursor.

This mutant strain is evaluated in a batch and fed-batch fermentation process in a 5L and 30L bioreactor as described in Example 1. In this example sucrose is used as a carbon source and lactose is added in the batch medium as precursor. Regular broth samples are taken, and sugars produced are measured as described in Example 1. UPLC analysis shows that fermentation broth of the selected strain taken at regular timepoints in fed-batch phase contains an oligosaccharide mixture comprising 2′FL, 3-FL, DiFL, 3′SL, 6′SL, di-SL, 3'S-2′FL, 3'S-3-FL, 6'S-2′FL, 6'S-3-FL, LNB, 2′FLNB, 4-FLNB, Di-FLNB, 3′ SLNB, 6′SLNB, LN3, 3'S-LN3, 6'S-LN3, LNT, LNFP-I, LSTa.

The resulting broth is clarified as described in example 14. In a separate example the cells were lysed to increase the release of oligosaccharides as described in Example 13 and further clarified as described in Example 14.

### Example 10. The Production of 2′Fucosyllactose in Fed Batch Fermentation

An E coli strain producing 2′-fucosyllactose as described in WO 2013087884A1 and further modified as described in WO 2021122708, or as described in Example 1, was used in a fed batch fermentation as described in Example 1. The fermentation medium contained 120 g/l of lactose and 100 g/l of sucrose in the batch medium and a 60% sucrose solution was fed to the bioreactor until the lactose concentration in the supernatant was lower than 5 g/l.

The medium composition described in example 1 was adapted wherein no ammonium chloride or ammonium sulphate is added to the medium, the sodium is added as sodium sulphate, hence reducing the amount of salts in the medium.

The final titer reached in the fermentation was 150 g/l.

The resulting broth is clarified as described in example 14. In a separate example the cells were lysed to increase the release of oligosaccharides as described in example 13 and further clarified as described in Example 14.

### Example 11. The Production of 6′Sialyllactose and 3′Sialyllactose in a Fed Batch Fermentation

An E coli strain producing 6′sialyllactose or 3′sialyllactose as described in WO 2018122225 was used in a fed batch fermentation as described in example 1. The fermentation medium contained 100 g/l of lactose and 60 g/l of sucrose and was fed with a 60% sucrose solution until the lactose concentration in the supernatant was lower than 5 g/l. The final titer reached in the fermentation was 100 g/l of either 6′ SL or 3′ SL.

The resulting broth is clarified as described in Example 14. In a separate example the cells were lysed to increase the release of oligosaccharides as described in Example 13 and further clarified as described in Example 14.

### Example 12. Composition Determination of the Cultivation or Fermentation Broth

For the cultivation or fermentation broths obtained in Examples 2-9 the composition was determined by measuring the Cell dry mass of the broth, the ash content of the supernatant and the broth, the oligosaccharide content of the supernatant and the broth and the total dry solids in the broth in accordance to the methods described in table 1. For all samples the total oligosaccharide content was below 80% on total dry solids. The oligosaccharide mixture purity in the broth ranged from 30% to 77%.

### Example 13. Cell Lysis

In many of the above described mutant strains the product is readily excreted from the cell. Larger molecules however tend to be released more difficult during the cultivation or fermentation process. Therefore, an additional step is optionally introduced to release the product from the cell. The broth from the fermentation processes of examples 2-8 is used in a cell lysis experiment.

A soft release of the product was established by heating for 1 h the broth to a temperature between 60° C. and 80° C. The higher the temperature, the more release was obtained, but color formation increased. The product release was most optimal at a pH below 6.5 and above 3. The least monosaccharide formation was found at a pH of above 3.9. The release of the product is quantified by the measured of the total oligosaccharide pool (cfr methods example 1) in the broth before and after treatment. When observing an increase in oligosaccharide concentration, the product is released from the cells.

To disrupt the cell integrity even more other methods are also commonly used, these are methods such as, freeze thawing and/or shear stress through sonication, mixing, a homogenizer and/or French press.

### Example 14. Broth Clarification

The broth originating from the cultivation or fermentation and, as the case may be, lysis step described in examples 2-13 are further clarified through microfiltration. For filtration several types of microfiltration membranes have been used to clarify the cultivation or fermentation broth with a pore size ranging between 0.1 to 10 μm (ceramic, PES, PVDF membranes). The membrane types were first used as dead-end filtration and further optimization was performed in cross flow filtration. The cross-flow microfiltration was followed by diafiltration to increase product yield after this purification step. The membranes are capable of separating large suspended solids such as colloids, particulates, fat, bacteria, yeasts, fungi, cells, while allowing sugars, proteins, salts, and low molecular weight molecules pass through the membrane.

The particle concentration in the filtrate was measured with a spectrophotometer through at light adsorption at 600 nm. This method allows the validation of particle removal and filtration optimization.

Alternative to microfiltration membranes, ultrafiltration membranes are used. Ultrafiltration membranes with a cut-off between 1000 Da and 10 kDa were tested (microdyne Nadir (3 kDa PES), Synder (3 kDa, PES), Synder Filtration MT (5 kDa, PES) and Synder Filtration ST (10 kDa, PES)). Alternative membranes with larger cut-offs will also work for broth clarification. The membranes were used in cross flow mode, and diafiltrations were applied similar to the microfiltration operation described above to increase product yield. The filtration efficiency is evaluated based on the particle concentration of the filtrate. Apart from cells and cell debris, membranes below 10 kDa efficiently remove DNA, protein and endotox, which were measured with the methods described in example 1. Higher cut-off membranes between 10 and 500 kDa remove cell mass efficiently, but do not retain smaller molecular weight products as efficiently, therefore requiring an additional Ultrafiltration step with a molecular weight cut-off below 10 kDa. A final recovery through ultrafiltration for broth clarification of Above 95% was obtained.

To enhance broth clarification through centrifugation, flocculants/coagulants have been used. Generally, Gypsum, Alum, calcium hydroxide, polyaluminum chloride, Aluminum chlorohydrate, are used as good flocculation agents. These flocculants were applied at a pH>7 and at temperatures between 4° C. and 20° C., more preferably between 4° C. and 10° C. pH<7 released toxic cations that are removed further through cation exchange. Alternative flocculants tested are based on polyacrylamide or biopolymer (chitosan), Floquant (SNF inc), Superfloc (Kemira) or hyperfloc (Hychem inc), Tramfloc. These flocculants were used in different concentrations: 0.05, 0.1 and 0.2 v/v % after diluting the broth 1:1 with RO-water, they were directly added to the broth and gently mixed for 10 minutes at room temperature. pH was kept at neutral conditions, between pH 6 and 7. At higher pH some degradation of the flocculant occurs, leading to compounds that are removed by means of ion exchange.

To test flocculation efficiency centrifugation was performed at 4000 g and the pellet strength and supernatant turbidity was evaluated after different centrifugation times. The oligosaccharide yield was measured by measuring the oligosaccharide supernatant concentration and the total supernatant volume. The pellet was washed several times to increase the release of oligosaccharides. A final oligosaccharide recovery between 90 and 98% was obtained.

### Example 15. Ultrafiltration

Ultrafiltration was performed on a Colossus apparatus (Convergence Industry, The Netherlands) controlled by a PC running Convergence Inspector software. Temperature, pressures and conductivity of both retentate and filtrate were measured inline, pH was measured offline with a calibrated pH probe (Hanna Instruments). The membrane to further remove DNA, protein and endotoxin was a 10 kDa membrane based on PES (Synder), used in crossflow. After filtration, the DNA, protein and endotoxin content was measured in the filtrate. The protein content was below 100 mg per kg dry solid, the DNA content below 10 ng per gram dry solid and the endotoxin was below 10000 EU per gram dry solid. No DNA from the production hosts could be detected in the filtrate.

Although in this example a polysulfon based membrane was used, other membrane materials will perform equally, these membrane materials can be a ceramic or made of a synthetic or natural polymer, e.g., polypropylene, cellulose acetate or polylactic acid from suppliers such as Synder, Tami, TriSep, Microdyn Nadir, GE.

### Example 16. Ion and Mono- and Disaccharide Removal Through Nanofiltration

Tangential flow nanofiltration was performed on a Colossus apparatus (Convergence Industry, The Netherlands) controlled by a PC running Convergence Inspector software. Temperature, pressures and conductivity of both retentate and filtrate were measured inline, pH was measured offline with a calibrated pH probe (Hanna Instruments). Clarified liquid treated with ultrafiltration from example 15 was further subjected to nanofiltration and sequential diafiltrations. To this end a polyamide base membrane with a cut off between 300 and 500 Da was used (TriSep XN-45 (TriSep Corporation, USA)) at 40° C. The diafiltrations were done with deionized water with a total volume of 5 times the volume of the oligosaccharide concentrate. This step reduced the disaccharide fraction on dry solid below 5% and reduced the total ash content of the liquid with 50%. The oligosaccharide concentration was increased to about 200 g/l.

### Example 17. Ion Removal Through Electrodialysis

The ED equipment used is a PCCell ED 64004 lab-scale ED stack, fitted with 5 cell pairs of the PC SA and PC SK standard ion-exchange membranes. The initial diluate and concentrate both comprised 1.5L of the feed stream obtained after the clarification and ultrafiltration in examples 14 and 15. The liquids obtained in these examples contained oligosaccharides and cations and anions with an ash content above 10% on dry solid. The desalination was done against a concentration gradient. Both streams are recirculated while a constant voltage of 7.5V is applied and the current and conductivity are monitored. Samples are taken at the beginning and end and periodically during the experiment. Water transport across the membranes is monitored by measuring the volume of all streams at the end of the experiment. To ensure efficient transfer of the current to the stack, an electrolyte solution of 60 g/L NaNO3 is recirculated at the electrodes.

The ED experiment was maintained until a stabilization of the current and conductivity was noticed. This indicates the point where desalination becomes slow and more inefficient. The conductivity decreases from 3.79 mS/cm in the feed to 0.88 mS/cm at the end of the experiment, indicating an overall desalination of 77%. The multivalent anions were removed up to 90%. The final oligosaccharide recovery was between 90 and 99%. The ash content on dry solid after electrodialysis was about 3% on dry solid.

### Example 18. Cation Ion Exchange

To remove unwanted ions cation exchange was performed. In the proton form this acidifies the liquid significantly, in the sodium, potassium, calcium, magnesium or other alkali metal form this creates an alkaline solution. Generally, oligosaccharides are considered to be unstable in such solutions, however good yields and stability have been found in the process when applying a single cation or sequential cation exchange step.

The clarified broths originating from examples 2 to 12 all were subjected after ultrafiltration as described in example 15 to a strong acid cation exchange resin containing column (1L of Amberlite IR120) in the proton form at a temperature of 10° C. The pH of the eluent of this cation exchange step was controlled by means of sodium or potassium hydroxide to keep the pH between 4 and 7. The oligosaccharide recovery was between 95 and 98% with little to no formation of monosaccharides, the Lead content was lower than 0.1 mg/kg dry solid, Arsenic: lower than 0.2 mg/kg dry solid, Cadmium lower than 0.1 mg/kg dry solid and Mercury was lower than 0.5 mg/kg dry solid.

The clarified broths originating from examples 3, 4, 5, 6, 9 and 11 all were subjected, after ultrafiltration as described in example 15, to a strong acid cation exchange resin containing column (1L of Amberlite FPC 22H) in the proton form at a temperature of 10° C. and then subjected to a strong cation exchange resin contain column (1L of Amberlite IR120) in the sodium form. The pH of the eluent of this cation exchange step was controlled by means of phosphoric acid, sulphuric acid, acetic acid, lactic acid, citric acid to keep the pH between 4 and 7. The oligosaccharide recovery was between 95 and 98% with little to no formation of monosaccharides, the Lead content was typically lower than 0.1 mg/kg dry solid, more typically lower than 0.05 mg/kg even more typically below 0.02 mg/kg, Arsenic content was typically lower than 0.2 mg/kg dry solid, more typically lower than 0.1 mg/kg, even more typically lower than 0.05 mg/kg, Cadmium content was typically lower than 0.1 mg/kg dry solid more typically lower than 0.05 mg/kg even more typically below 0.02 mg/kg, and Mercury was typically lower than 0.5 mg/kg dry solid, more typically lower than 0.2 mg/kg even more typically below 0.1 mg/kg.

### Example 19. Concentration Through Nanofiltration

Nanofiltration was carried out with an NF-2540 membrane (DOW) with a cut off of 200 Da to concentrate the de-ionized solutions after ion exchange, electrodialysis or nanofiltration up to 25 Brix. During the filtration process a pressure across the membrane in the range of 20-25 bar was used and a process temperature of 45° C. The solution was continuous recirculated over the membrane for concentration, leading to a dry matter content of the concentrate up to 25% Brix.

To remove some of the monosaccharides formed during the cation exchange step, typically a nanofiltration step of 300 to 500 Da is used at a membrane pressure of 20 to 25 bar and at a temperature above 30° C. The membrane allows concentrating the oligosaccharide solution to about 15 to 20% Brix.

### Example 20. Color Removal

To achieve decolorization, several samples throughout the process were subjected to activated charcoal treatment with Norit SX PLUS activated charcoal (0.5% m/v). Color removal was measured with a spectrophotometer at 420 nm. In all samples the color intensity at 420 nm was reduced 50 to 100 fold.

### Example 21. Spray Drying of Oligosaccharide Mixture

A mixture of oligosaccharides at different concentrations was spray dried with pilot spray dry equipment. The equipment had an evaporation capacity of 25 kg/h.

For spray drying the liquid was heated to a temperature between 50 and 100° C., to lower the viscosity. The pH of the liquid was set to a pH of 4 to 6. More preferably the pH is set to 4 to 5 and temperatures are kept between 50 and 70° C.

The oligosaccharide concentration in the feed is between 20% and 80% brix. These concentrations were obtained by rotary evaporation or wiped film evaporation. The concentrated liquids were fed to the spray dryer at a rate between 50 and 90%. The higher the percentage brix, the faster the feed rate.

The used inlet temperature ranged between 120 and 280° C. The outlet temperature ranged between 100° C. and 180° C. The atomizer wheel rotation speed was set between 10,000 and 28,000 rpm. In one specific test. the inlet temperature was set at 184° C., outlet temperature was set at 110° C., and atomizer rate was set at 21,500 rpm.

The obtained powder had a white to off white color and the pH after redissolving water at a concentration of 10%, the pH was between 4 and 6. The purity of the oligosaccharide mixture was above 80% of oligosaccharides on dry solid. The spray dried oligosaccharide mixtures had about 3 to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 5% (on total dry solid), the Lead content was typically lower than 0.1 mg/kg dry solid, more typically lower than 0.05 mg/kg even more typically below 0.02 mg/kg, Arsenic content was typically lower than 0.2 mg/kg dry solid, more typically lower than mg/kg, even more typically lower than 0.05 mg/kg, Cadmium content was typically lower than 0.1 mg/kg dry solid more typically lower than 0.05 mg/kg even more typically below 0.02 mg/kg, and Mercury was typically lower than 0.5 mg/kg dry solid, more typically lower than 0.2 mg/kg even more typically below 0.1 mg/kg.

### Example 22. Stepwise Purification of a Fucosylated Oligosaccharide Mixture

The broth of example 8 was clarified by first applying microfiltration with a 0.45 μm pore sized membrane, removing biomass at 60° C. and a pH of 4 to 5. The filtrate of the microfiltration step was in a second step subjected to ultrafiltration in which a PES membrane of kDa was used, removing protein, endotoxin and DNA. The resulting filtrate was further concentrated by nanofiltration, partially removing salts and disaccharides from the liquid with a polyamide membrane of 300 to 500 Da at 40° C. In the nanofiltration step the oligosaccharide mixture was concentrated to a concentration of about 200 g/l or 20 Brix. The resulting concentrate was further decolored by means of activated charcoal and cations were removed by a cation exchange step resulting in an ash content below 5% on dry mass. This liquid was set to a pH between 5 and 7 and concentrated by means of evaporation to about 50 brix. The final solution was spray dried with an inlet temperature of 160° C., outlet temperature of 75° C., an airflow of 600 L/h and a feed rate of 8 ml/min on a Procept spray dryer. The obtained powder had a white to off white color and the pH after redissolving water at a concentration of 10%, the pH was between 4 and 6. The purity of the oligosaccharide mixture was above 80% of oligosaccharides on dry solid. The spray dried oligosaccharide mixtures had about 3 to 10% of water content, the protein content was below 100 mg per kg dry solid, the DNA content below ng per gram dry solid and the endotoxin was below 10000 EU per gram dry solid. No DNA from the production hosts could be detected in the filtrate. The ash content after treatment was below 5% (on total dry solid), the Lead content was lower than 0.1 mg/kg dry solid, Arsenic: lower than 0.2 mg/kg dry solid, Cadmium lower than 0.1 mg/kg dry solid and Mercury was lower than 0.5 mg/kg dry solid. The oligosaccharides present in the obtained powder are 2′FL, 3-FL, DiFL, LN3, LNT and LNFP-I.

### Example 23. Stepwise Purification of a Sialylated Oligosaccharide Mixture

The broth of example 8 was clarified by first applying microfiltration with a 0.45 μm pore sized membrane, removing biomass at 60° C. and a pH of 4 to 5. The filtrate of the microfiltration step was in a second step subjected to ultrafiltration in which a PES membrane of kDa was used, removing protein, endotoxin and DNA. The resulting filtrate was further concentrated and deionized with electrodialysis resulting into a liquid solution with a conductivity of about 0.9 mS/cm. After the electrodialysis step the retentate is further treated in a nanofiltration step concentrating the oligosaccharide mixture to a concentration of about 200 g/l or 20 Brix. The resulting concentrate was further decolored by means of activated charcoal and treated with a cation exchange step in an ash content below 8% on dry mass. This liquid was set to a pH between 5 and 7 and concentrated by means of evaporation to about 50 brix. The final solution was spray dried with an inlet temperature of 160° C., outlet temperature of 75° C., an airflow of 600 L/h and a feed rate of 8 ml/min on a Procept spray dryer. The obtained powder had a white to off white color and the pH after redissolving water at a concentration of 10%, the pH was between 4 and 6. The purity of the oligosaccharide mixture was above 80% of oligosaccharides on dry solid. The spray dried oligosaccharide mixtures had about 3 to 10% of water content, the protein content was below 100 mg per kg dry solid, the DNA content below 10 ng per gram dry solid and the endotoxin was below 10000 EU per gram dry solid. No DNA from the production hosts could be detected in the filtrate. The ash content after treatment was below 5% (on total dry solid), the Lead content was lower than 0.02 mg/kg dry solid, Arsenic: lower than 0.05 mg/kg dry solid, Cadmium lower than 0.05 mg/kg dry solid and Mercury was lower than 0.1 mg/kg dry solid.

### Example 24. Examples of Stepwise Implementations of this Disclosure

Each of the steps described hereunder must be understood as described herein.

Purification of an oligosaccharide mixture obtained from a cultivation or fermentation process, comprising the following steps 1) broth clarification, 2) ultrafiltration, 3) nanofiltration, 4) cation exchange, 5) concentration, and 6) spray drying.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, and 6) spray drying.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, and 6) spray drying.

Purification of an oligosaccharide mixture obtained from a cultivation or fermentation process, comprising the following steps 1) broth clarification through ultrafiltration, 2) nanofiltration, 3) cation exchange, 4) concentration, and 5) spray drying.

Purification of an oligosaccharide mixture obtained from a cultivation or fermentation process, comprising the following steps 1) broth clarification, 2) ultrafiltration, 3) electrodialysis, 4) cation exchange, 5) concentration, and 6) spray drying.

Purification of an oligosaccharide mixture obtained from a cultivation or microfiltration, 2) ultrafiltration, 3) electrodialysis, 4) cation exchange, 5) concentration, and 6) spray drying.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, and 6) spray drying.

Purification of an oligosaccharide mixture obtained from a cultivation or fermentation process, comprising the following steps 1) broth clarification through ultrafiltration, 2) electrodialysis, 3) cation exchange, 4) concentration, and 5) spray drying.

Purification of an oligosaccharide mixture obtained from a cultivation or fermentation process, comprising the following steps 1) broth clarification, 2) ultrafiltration, 3) nanofiltration, 4) cation exchange, 5) concentration, 6) Activated Charcoal treatment, and 7) spray drying.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) spray drying.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) spray drying.

Purification of an oligosaccharide mixture obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) spray drying.

Purification of an oligosaccharide mixture obtained from a cultivation or fermentation process, comprising the following steps 1) broth clarification, 2) ultrafiltration, 3) electrodialysis, 4) cation exchange, 5) concentration, 6) Activated Charcoal treatment, and 7) spray drying.

Purification of an oligosaccharide mixture obtained from a cultivation or microfiltration, 2) ultrafiltration, 3) electrodialysis, 4) cation exchange, 5) concentration, 6) Activated Charcoal treatment, and 7) spray drying.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) spray drying.

Purification of an oligosaccharide mixture obtained from a cultivation or fermentation process, comprising the following steps 1) broth clarification through ultrafiltration, 4) cation exchange, 5) concentration, 6) Activated Charcoal treatment and 7) spray drying.

Purification of an oligosaccharide mixture obtained from a cultivation or fermentation process, comprising the following steps 1) broth clarification, 2) ultrafiltration, 3) cation exchange, 4) concentration, and 5) spray drying.

Purification of an oligosaccharide mixture obtained from a cultivation or fermentation process, comprising the following steps 1) broth clarification through microfiltration, 2) ultrafiltration, 3) cation exchange, 4) concentration, and 5) spray drying.

Purification of an oligosaccharide mixture obtained from a cultivation or fermentation process, comprising the following steps 1) broth clarification through centrifugation, 2) ultrafiltration, 3) cation exchange, 4) concentration, and 5) spray drying.

Purification of an oligosaccharide mixture obtained from a cultivation or fermentation process, comprising the following steps 1) broth clarification through ultrafiltration, 2) cation exchange, 3) concentration, and 4) spray drying.

Purification of an oligosaccharide mixture obtained from a cultivation or fermentation process, comprising the following steps 1) broth clarification, 2) ultrafiltration, 3) cation exchange, 4) concentration, 5) Activated Charcoal treatment, and 6) spray drying.

Purification of an oligosaccharide mixture obtained from a cultivation or fermentation process, comprising the following steps 1) broth clarification through microfiltration, 2) ultrafiltration, 3) cation exchange, 4) concentration, 5) Activated Charcoal treatment, and 6) spray drying.

Purification of an oligosaccharide mixture obtained from a cultivation or centrifugation, 2) ultrafiltration, 3) cation exchange, 4) concentration, 5) Activated Charcoal treatment, and 6) spray drying.

Purification of an oligosaccharide mixture obtained from a cultivation or fermentation process, comprising the following steps 1) broth clarification through ultrafiltration, 2) cation exchange, 3) concentration, 4) Activated Charcoal treatment, and 5) spray drying.

Purification of an oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) spray drying.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) spray drying.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) spray drying.

Purification of an oligosaccharide mixture obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) spray drying.

Purification of an oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) spray drying.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) spray drying.

Purification of an oligosaccharide mixture obtained from a cultivation or centrifugation, 2) ultrafiltration, 3) electrodialysis, 4) cation exchange, 5) concentration, monosaccharide and disaccharide removal through nanofiltration, and 6) spray drying.

Purification of an oligosaccharide mixture obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) spray drying.

Purification of an oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) spray drying.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) spray drying.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) spray drying.

Purification of an oligosaccharide mixture obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) spray drying.

Purification of an oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) spray drying.

Purification of an oligosaccharide mixture obtained from a cultivation or microfiltration, 2) ultrafiltration, 3) electrodialysis, 4) cation exchange, 5) concentration, monosaccharide and disaccharide removal through nanofiltration, 6) Activated Charcoal treatment, and 7) spray drying.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) spray drying.

Purification of an oligosaccharide mixture obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) spray drying.

Purification of an oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, and 5) spray drying.

Purification of an oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) spray drying.

Purification of an oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) spray drying.

Purification of an oligosaccharide mixture obtained from a cultivation or fermentation process, comprising the following steps 1) broth clarification through ultrafiltration, 2) cation exchange, 3) concentration, monosaccharide and disaccharide removal through nanofiltration, and 4) spray drying.

Purification of an oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) spray drying.

Purification of an oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) spray drying.

Purification of an oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) spray drying.

Purification of an oligosaccharide mixture obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) spray drying.

Purification of an oligosaccharide mixture obtained from a cultivation or fermentation process, comprising the following steps 1) broth clarification, 2) ultrafiltration, 3) nanofiltration, 4) cation exchange, 5) concentration, and 6) lyophilization.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, and 6) lyophilization.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, and 6) lyophilization.

Purification of an oligosaccharide mixture obtained from a cultivation or fermentation process, comprising the following steps 1) broth clarification through ultrafiltration, 2) nanofiltration, 3) cation exchange, 4) concentration, and 5) lyophilization.

Purification of an oligosaccharide mixture obtained from a cultivation or fermentation process, comprising the following steps 1) broth clarification, 2) ultrafiltration, 3) electrodialysis, 4) cation exchange, 5) concentration, and 6) lyophilization.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, and 6) lyophilization.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, and 6) lyophilization.

Purification of an oligosaccharide mixture obtained from a cultivation or fermentation process, comprising the following steps 1) broth clarification through ultrafiltration, 2) electrodialysis, 3) cation exchange, 4) concentration, and 5) lyophilization.

Purification of an oligosaccharide mixture obtained from a cultivation or fermentation process, comprising the following steps 1) broth clarification, 2) ultrafiltration, 3) nanofiltration, 4) cation exchange, 5) concentration, 6) Activated Charcoal treatment, and 7) lyophilization.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, 6) Activated Charcoal treatment 7) lyophilization.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) lyophilization.

Purification of an oligosaccharide mixture obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) lyophilization.

Purification of an oligosaccharide mixture obtained from a cultivation or fermentation process, comprising the following steps 1) broth clarification, 2) ultrafiltration, 3) electrodialysis, 4) cation exchange, 5) concentration, 6) Activated Charcoal treatment, and 7) lyophilization.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) lyophilization.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) lyophilization.

Purification of an oligosaccharide mixture obtained from a cultivation or fermentation process, comprising the following steps 1) broth clarification through ultrafiltration, 4) cation exchange, 5) concentration, 6) Activated Charcoal treatment, and 7) lyophilization.

Purification of an oligosaccharide mixture obtained from a cultivation or fermentation process, comprising the following steps 1) broth clarification, 2) ultrafiltration, 3) cation exchange, 4) concentration, and 5) lyophilization.

Purification of an oligosaccharide mixture obtained from a cultivation or fermentation process, comprising the following steps 1) broth clarification through microfiltration, 2) ultrafiltration, 3) cation exchange, 4) concentration, and 5) lyophilization.

Purification of an oligosaccharide mixture obtained from a cultivation or fermentation process, comprising the following steps 1) broth clarification through centrifugation, 2) ultrafiltration, 3) cation exchange, 4) concentration, and 5) lyophilization.

Purification of an oligosaccharide mixture obtained from a cultivation or fermentation process, comprising the following steps 1) broth clarification through ultrafiltration, 2) cation exchange, 3) concentration, and 4) lyophilization.

Purification of an oligosaccharide mixture obtained from a cultivation or fermentation process, comprising the following steps 1) broth clarification, 2) ultrafiltration, 3) cation exchange, 4) concentration, 5) Activated Charcoal treatment and 6) lyophilization.

Purification of an oligosaccharide mixture obtained from a cultivation or fermentation process, comprising the following steps 1) broth clarification through microfiltration, 2) ultrafiltration, 3) cation exchange, 4) concentration, 5) Activated Charcoal treatment and 6) lyophilization.

Purification of an oligosaccharide mixture obtained from a cultivation or fermentation process, comprising the following steps 1) broth clarification through centrifugation, 2) ultrafiltration, 3) cation exchange, 4) concentration, 5) Activated Charcoal treatment, and 6) lyophilization.

Purification of an oligosaccharide mixture obtained from a cultivation or fermentation process, comprising the following steps 1) broth clarification through ultrafiltration, 2) cation exchange, 3) concentration, 4) Activated Charcoal treatment, and 5) lyophilization.

Purification of an oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) lyophilization.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) lyophilization.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) lyophilization.

Purification of an oligosaccharide mixture obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) lyophilization.

Purification of an oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) lyophilization.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) lyophilization.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) lyophilization.

Purification of an oligosaccharide mixture obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) lyophilization.

Purification of an oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) lyophilization.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) lyophilization.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) lyophilization.

Purification of an oligosaccharide mixture obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) lyophilization.

Purification of an oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) lyophilization.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) lyophilization.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) lyophilization.

Purification of an oligosaccharide mixture obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) lyophilization.

Purification of an oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, and 5) lyophilization.

Purification of an oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) lyophilization.

Purification of an oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) lyophilization.

Purification of an oligosaccharide mixture obtained from a cultivation or fermentation process, comprising the following steps 1) broth clarification through ultrafiltration, 2) cation exchange, 3) concentration, monosaccharide and disaccharide removal through nanofiltration, and 4) lyophilization.

Purification of an oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) lyophilization.

Purification of an oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) lyophilization.

Purification of an oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) lyophilization.

Purification of an oligosaccharide mixture obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) lyophilization.

Purification of an oligosaccharide mixture obtained from a cultivation or fermentation process, comprising the following steps 1) broth clarification, 2) ultrafiltration, 3) nanofiltration, 4) cation exchange, 5) concentration, and 6) concentrating to a syrup of at least 40% dry matter.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, and 6) concentrating to a syrup of at least 40% dry matter.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, and 6) concentrating to a syrup of at least 40% dry matter.

Purification of an oligosaccharide mixture obtained from a cultivation or fermentation process, comprising the following steps 1) broth clarification through ultrafiltration, 2) nanofiltration, 3) cation exchange, 4) concentration, and 5) concentrating to a syrup of at least 40% dry matter.

Purification of an oligosaccharide mixture obtained from a cultivation or fermentation process, comprising the following steps 1) broth clarification, 2) ultrafiltration, 3) electrodialysis, 4) cation exchange, 5) concentration, and 6) concentrating to a syrup of at least 40% dry matter.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, and 6) concentrating to a syrup of at least 40% dry matter.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, and 6) concentrating to a syrup of at least 40% dry matter.

Purification of an oligosaccharide mixture obtained from a cultivation or fermentation process, comprising the following steps 1) broth clarification through ultrafiltration, 2) electrodialysis, 3) cation exchange, 4) concentration, and 5) concentrating to a syrup of at least 40% dry matter.

Purification of an oligosaccharide mixture obtained from a cultivation or fermentation process, comprising the following steps 1) broth clarification, 2) ultrafiltration, 3) nanofiltration, 4) cation exchange, 5) concentration, 6) Activated Charcoal treatment, and 7) concentrating to a syrup of at least 40% dry matter.

Purification of an oligosaccharide mixture obtained from a cultivation or microfiltration, 2) ultrafiltration, 3) nanofiltration, 4) cation exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification, 2) ultrafiltration, 3) electrodialysis, 4) cation exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification through ultrafiltration, 4) cation exchange, 5) concentration, 6) Activated Charcoal treatment, and 7) concentrating to a syrup of at least 40% dry matter.

Purification of an oligosaccharide mixture obtained from a cultivation or fermentation process, comprising the following steps 1) broth clarification, 2) ultrafiltration, 3) cation exchange, 4) concentration, and 5) concentrating to a syrup of at least 40% dry matter.

Purification of an oligosaccharide mixture obtained from a cultivation or microfiltration, 2) ultrafiltration, 3) cation exchange, 4) concentration, and 5) concentrating to a syrup of at least 40% dry matter.

Purification of an oligosaccharide mixture obtained from a cultivation or fermentation process, comprising the following steps 1) broth clarification through centrifugation, 2) ultrafiltration, 3) cation exchange, 4) concentration, and 5) concentrating to a syrup of at least 40% dry matter.

Purification of an oligosaccharide mixture obtained from a cultivation or fermentation process, comprising the following steps 1) broth clarification through ultrafiltration, 2) cation exchange, 3) concentration, and 4) concentrating to a syrup of at least 40% dry matter.

Purification of an oligosaccharide mixture obtained from a cultivation or fermentation process, comprising the following steps 1) broth clarification, 2) ultrafiltration, 3) cation exchange, 4) concentration, 5) Activated Charcoal treatment, and 6) concentrating to a syrup of at least 40% dry matter.

Purification of an oligosaccharide mixture obtained from a cultivation or fermentation process, comprising the following steps 1) broth clarification through microfiltration, 2) ultrafiltration, 3) cation exchange, 4) concentration, 5) Activated Charcoal treatment, and 6) concentrating to a syrup of at least 40% dry matter.

Purification of an oligosaccharide mixture obtained from a cultivation or fermentation process, comprising the following steps 1) broth clarification through centrifugation, 2) ultrafiltration, 3) cation exchange, 4) concentration, 5) Activated Charcoal treatment, and 6) concentrating to a syrup of at least 40% dry matter.

Purification of an oligosaccharide mixture obtained from a cultivation or fermentation process, comprising the following steps 1) broth clarification through ultrafiltration, 2) cation exchange, 3) concentration, 4) Activated Charcoal treatment, and 5) concentrating to a syrup of at least 40% dry matter.

Purification of an oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) concentrating to a syrup of at least 40% dry matter.

Purification of an oligosaccharide mixture obtained from a cultivation or microfiltration, 2) ultrafiltration, 3) nanofiltration, 4) cation exchange, 5) concentration, monosaccharide and disaccharide removal through nanofiltration, and 6) concentrating to a syrup of at least 40% dry matter.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) concentrating to a syrup of at least 40% dry matter.

Purification of an oligosaccharide mixture obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) concentrating to a syrup of at least 40% dry matter.

Purification of an oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) concentrating to a syrup of at least 40% dry matter.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) concentrating to a syrup of at least 40% dry matter.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) concentrating to a syrup of at least 40% dry matter.

Purification of an oligosaccharide mixture obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) concentrating to a syrup of at least 40% dry matter.

Purification of an oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) concentrating to a syrup of at least 40% dry matter.

Purification of an oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) concentrating to a syrup of at least 40% dry matter.

Purification of an oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) concentrating to a syrup of at least 40% dry matter.

Purification of an oligosaccharide mixture obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) concentrating to a syrup of at least 40% dry matter.

Purification of an oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) concentrating to a syrup of at least 40% dry matter.

Purification of an oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) concentrating to a syrup of at least 40% dry matter.

Purification of an oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) concentrating to a syrup of at least 40% dry matter.

Purification of an oligosaccharide mixture obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) concentrating to a syrup of at least 40% dry matter.

Purification of an oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, and 5) concentrating to a syrup of at least 40% dry matter.

Purification of an oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) concentrating to a syrup of at least 40% dry matter.

Purification of an oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) concentrating to a syrup of at least 40% dry matter.

Purification of an oligosaccharide mixture obtained from a cultivation or fermentation process, comprising the following steps 1) broth clarification through ultrafiltration, 2) cation exchange, 3) concentration, monosaccharide and disaccharide removal through nanofiltration, and 4) concentrating to a syrup of at least 40% dry matter.

Purification of an oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) concentrating to a syrup of at least 40% dry matter.

Purification of an oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) concentrating to a syrup of at least 40% dry matter.

Purification of an oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) concentrating to a syrup of at least 40% dry matter.

Purification of an oligosaccharide mixture obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) concentrating to a syrup of at least 40% dry matter.

Purification of a single oligosaccharide obtained from a cultivation or fermentation process, comprising the following steps 1) broth clarification, 2) ultrafiltration, 3) nanofiltration, 4) cation exchange, 5) concentration, and 6) crystallization.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, and 6) crystallization.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, and 6) crystallization.

Purification of a single oligosaccharide obtained from a cultivation or fermentation process, comprising the following steps 1) broth clarification through ultrafiltration, 2) nanofiltration, 3) cation exchange, 4) concentration, and 5) crystallization.

Purification of a single oligosaccharide obtained from a cultivation or fermentation process, comprising the following steps 1) broth clarification, 2) ultrafiltration, 3) electrodialysis, 4) cation exchange, 5) concentration, and 6) crystallization.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, and 6) crystallization.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, and 6) crystallization.

Purification of a single oligosaccharide obtained from a cultivation or fermentation process, comprising the following steps 1) broth clarification through ultrafiltration, 2) electrodialysis, 3) cation exchange, 4) concentration, and 5) crystallization.

Purification of a single oligosaccharide obtained from a cultivation or fermentation process, comprising the following steps 1) broth clarification, 2) ultrafiltration, 3) nanofiltration, 4) cation exchange, 5) concentration, 6) Activated Charcoal treatment, and 7) crystallization.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) crystallization.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) crystallization.

Purification of a single oligosaccharide obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) crystallization.

Purification of a single oligosaccharide obtained from a cultivation or fermentation process, comprising the following steps 1) broth clarification, 2) ultrafiltration, 3) electrodialysis, 4) cation exchange, 5) concentration, 6) Activated Charcoal treatment, and 7) crystallization.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) crystallization.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) crystallization.

Purification of a single oligosaccharide obtained from a cultivation or fermentation process, comprising the following steps 1) broth clarification through ultrafiltration, 4) cation exchange, 5) concentration, 6) Activated Charcoal treatment, and 7) crystallization.

Purification of a single oligosaccharide obtained from a cultivation or fermentation process, comprising the following steps 1) broth clarification, 2) ultrafiltration, 3) cation exchange, 4) concentration, and 5) crystallization.

Purification of a single oligosaccharide obtained from a cultivation or fermentation process, comprising the following steps 1) broth clarification through microfiltration, 2) ultrafiltration, 3) cation exchange, 4) concentration, and 5) crystallization.

Purification of a single oligosaccharide obtained from a cultivation or fermentation process, comprising the following steps 1) broth clarification through centrifugation, 2) ultrafiltration, 3) cation exchange, 4) concentration, and 5) crystallization.

Purification of a single oligosaccharide obtained from a cultivation or fermentation process, comprising the following steps 1) broth clarification through ultrafiltration, 2) cation exchange, 3) concentration, and 4) crystallization.

Purification of a single oligosaccharide obtained from a cultivation or fermentation process, comprising the following steps 1) broth clarification, 2) ultrafiltration, 3) cation exchange, 4) concentration, 5) Activated Charcoal treatment, and 6) crystallization.

Purification of a single oligosaccharide obtained from a cultivation or fermentation process, comprising the following steps 1) broth clarification through microfiltration, 2) ultrafiltration, 3) cation exchange, 4) concentration, 5) Activated Charcoal treatment, and 6) crystallization.

Purification of a single oligosaccharide obtained from a cultivation or fermentation process, comprising the following steps 1) broth clarification through centrifugation, 2) ultrafiltration, 3) cation exchange, 4) concentration, 5) Activated Charcoal treatment, and 6) crystallization.

Purification of a single oligosaccharide obtained from a cultivation or fermentation process, comprising the following steps 1) broth clarification through ultrafiltration, 2) cation exchange, 3) concentration, 4) Activated Charcoal treatment, and 5) crystallization.

Purification of a single oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) crystallization.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) crystallization.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) crystallization.

Purification of a single oligosaccharide obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) crystallization.

Purification of a single oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) crystallization.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) crystallization.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) crystallization.

Purification of a single oligosaccharide obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) crystallization.

Purification of a single oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) crystallization.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) crystallization.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) crystallization.

Purification of a single oligosaccharide obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) crystallization.

Purification of a single oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) crystallization.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) crystallization.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) crystallization.

Purification of a single oligosaccharide obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) crystallization.

Purification of a single oligosaccharide obtained from a cultivation or fermentation process, comprising the following steps 1) broth clarification, 2) ultrafiltration, 3) cation exchange, 4) concentration, monosaccharide and disaccharide removal through nanofiltration, and 5) crystallization.

Purification of a single oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) crystallization.

Purification of a single oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) crystallization.

Purification of a single oligosaccharide obtained from a cultivation or fermentation process, comprising the following steps 1) broth clarification through ultrafiltration, 2) cation exchange, 3) concentration, monosaccharide and disaccharide removal through nanofiltration, and 4) crystallization.

Purification of a single oligosaccharide obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) crystallization.

Purification of a single oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) crystallization.

Purification of a single oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) crystallization.

Purification of a single oligosaccharide obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) crystallization.

Purification of a single oligosaccharide obtained from a cultivation or fermentation process, comprising the following steps 1) broth clarification, 2) ultrafiltration, 3) nanofiltration, 4) cation exchange, 5) concentration, and 6) concentrating to a syrup of at least 40% dry matter.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, and 6) concentrating to a syrup of at least 40% dry matter.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, and 6) concentrating to a syrup of at least 40% dry matter.

Purification of a single oligosaccharide obtained from a cultivation or fermentation process, comprising the following steps 1) broth clarification through ultrafiltration, 2) nanofiltration, 3) cation exchange, 4) concentration, and 5) concentrating to a syrup of at least 40% dry matter.

Purification of a single oligosaccharide obtained from a cultivation or fermentation process, comprising the following steps 1) broth clarification, 2) ultrafiltration, 3) electrodialysis, 4) cation exchange, 5) concentration, and 6) concentrating to a syrup of at least 40% dry matter.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, and 6) concentrating to a syrup of at least 40% dry matter.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, and 6) concentrating to a syrup of at least 40% dry matter.

Purification of a single oligosaccharide obtained from a cultivation or fermentation process, comprising the following steps 1) broth clarification through ultrafiltration, 2) electrodialysis, 3) cation exchange, 4) concentration, and 5) concentrating to a syrup of at least 40% dry matter.

Purification of a single oligosaccharide obtained from a cultivation or fermentation process, comprising the following steps 1) broth clarification, 2) ultrafiltration, 3) nanofiltration, 4) cation exchange, 5) concentration, 6) Activated Charcoal treatment, and 7) concentrating to a syrup of at least 40% dry matter.

Purification of a single oligosaccharide obtained from a cultivation or microfiltration, 2) ultrafiltration, 3) nanofiltration, 4) cation exchange, 5) concentration, 6) Activated Charcoal treatment, and 7) concentrating to a syrup of at least 40% dry matter.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) concentrating to a syrup of at least 40% dry matter.

Purification of a single oligosaccharide obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) concentrating to a syrup of at least 40% dry matter.

Purification of a single oligosaccharide obtained from a cultivation or fermentation process, comprising the following steps 1) broth clarification, 2) ultrafiltration, 3) electrodialysis, 4) cation exchange, 5) concentration, 6) Activated Charcoal treatment, and 7) concentrating to a syrup of at least 40% dry matter.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) concentrating to a syrup of at least 40% dry matter.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) concentrating to a syrup of at least 40% dry matter.

Purification of a single oligosaccharide obtained from a cultivation or fermentation process, comprising the following steps 1) broth clarification through ultrafiltration, 4) cation exchange, 5) concentration, 6) Activated Charcoal treatment, and 7) concentrating to a syrup of at least 40% dry matter.

Purification of a single oligosaccharide obtained from a cultivation or fermentation process, comprising the following steps 1) broth clarification, 2) ultrafiltration, 3) cation exchange, 4) concentration, and 5) concentrating to a syrup of at least 40% dry matter.

Purification of a single oligosaccharide obtained from a cultivation or microfiltration, 2) ultrafiltration, 3) cation exchange, 4) concentration, and 5) concentrating to a syrup of at least 40% dry matter.

Purification of a single oligosaccharide obtained from a cultivation or fermentation process, comprising the following steps 1) broth clarification through centrifugation, 2) ultrafiltration, 3) cation exchange, 4) concentration, and 5) concentrating to a syrup of at least 40% dry matter.

Purification of a single oligosaccharide obtained from a cultivation or fermentation process, comprising the following steps 1) broth clarification through ultrafiltration, 2) cation exchange, 3) concentration, and 4) concentrating to a syrup of at least 40% dry matter.

Purification of a single oligosaccharide obtained from a cultivation or fermentation process, comprising the following steps 1) broth clarification, 2) ultrafiltration, 3) cation exchange, 4) concentration, 5) Activated Charcoal treatment, and 6) concentrating to a syrup of at least 40% dry matter.

Purification of a single oligosaccharide obtained from a cultivation or fermentation process, comprising the following steps 1) broth clarification through microfiltration, 2) ultrafiltration, 3) cation exchange, 4) concentration, 5) Activated Charcoal treatment, and 6) concentrating to a syrup of at least 40% dry matter.

Purification of a single oligosaccharide obtained from a cultivation or fermentation process, comprising the following steps 1) broth clarification through centrifugation, 2) ultrafiltration, 3) cation exchange, 4) concentration, 5) Activated Charcoal treatment, and 6) concentrating to a syrup of at least 40% dry matter.

Purification of a single oligosaccharide obtained from a cultivation or fermentation process, comprising the following steps 1) broth clarification through ultrafiltration, 2) cation exchange, 3) concentration, 4) Activated Charcoal treatment, and 5) concentrating to a syrup of at least 40% dry matter.

Purification of a single oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) concentrating to a syrup of at least 40% dry matter.

Purification of a single oligosaccharide obtained from a cultivation or microfiltration, 2) ultrafiltration, 3) nanofiltration, 4) cation exchange, 5) concentration, monosaccharide and disaccharide removal through nanofiltration, and 6) concentrating to a syrup of at least 40% dry matter.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) concentrating to a syrup of at least 40% dry matter.

Purification of a single oligosaccharide obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) concentrating to a syrup of at least 40% dry matter.

Purification of a single oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) concentrating to a syrup of at least 40% dry matter.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) concentrating to a syrup of at least 40% dry matter.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) concentrating to a syrup of at least 40% dry matter.

Purification of a single oligosaccharide obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) concentrating to a syrup of at least 40% dry matter.

Purification of a single oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) concentrating to a syrup of at least 40% dry matter.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) concentrating to a syrup of at least 40% dry matter.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) concentrating to a syrup of at least 40% dry matter.

Purification of a single oligosaccharide obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) concentrating to a syrup of at least 40% dry matter.

Purification of a single oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) concentrating to a syrup of at least 40% dry matter.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) concentrating to a syrup of at least 40% dry matter.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) concentrating to a syrup of at least 40% dry matter.

Purification of a single oligosaccharide obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) concentrating to a syrup of at least 40% dry matter.

Purification of a single oligosaccharide obtained from a cultivation or fermentation process, comprising the following steps 1) broth clarification, 2) ultrafiltration, 3) cation exchange, 4) concentration, monosaccharide and disaccharide removal through nanofiltration, and 5) concentrating to a syrup of at least 40% dry matter.

Purification of a single oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) concentrating to a syrup of at least 40% dry matter.

Purification of a single oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) concentrating to a syrup of at least 40% dry matter.

Purification of a single oligosaccharide obtained from a cultivation or fermentation process, comprising the following steps 1) broth clarification through ultrafiltration, 2) cation exchange, 3) concentration, monosaccharide and disaccharide removal through nanofiltration, and 4) concentrating to a syrup of at least 40% dry matter.

Purification of a single oligosaccharide obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) concentrating to a syrup of at least 40% dry matter.

Purification of a single oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) concentrating to a syrup of at least 40% dry matter.

Purification of a single oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) concentrating to a syrup of at least 40% dry matter.

Purification of a single oligosaccharide obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) concentrating to a syrup of at least 40% dry matter.

Purification of a single oligosaccharide obtained from a cultivation or fermentation process, comprising the following steps 1) broth clarification, 2) ultrafiltration, 3) nanofiltration, 4) cation exchange, 5) concentration, and 6) lyophilization.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, and 6) lyophilization.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, and 6) lyophilization.

Purification of a single oligosaccharide obtained from a cultivation or fermentation process, comprising the following steps 1) broth clarification through ultrafiltration, 2) nanofiltration, 3) cation exchange, 4) concentration, and 5) lyophilization.

Purification of a single oligosaccharide obtained from a cultivation or fermentation process, comprising the following steps 1) broth clarification, 2) ultrafiltration, 3) electrodialysis, 4) cation exchange, 5) concentration, and 6) lyophilization.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, and 6) lyophilization.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, and 6) lyophilization.

Purification of a single oligosaccharide obtained from a cultivation or fermentation process, comprising the following steps 1) broth clarification through ultrafiltration, 2) electrodialysis, 3) cation exchange, 4) concentration, and 5) lyophilization.

Purification of a single oligosaccharide obtained from a cultivation or fermentation process, comprising the following steps 1) broth clarification, 2) ultrafiltration, 3) nanofiltration, 4) cation exchange, 5) concentration, 6) Activated Charcoal treatment, and 7) lyophilization.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) lyophilization.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) lyophilization.

Purification of a single oligosaccharide obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) lyophilization.

Purification of a single oligosaccharide obtained from a cultivation or fermentation process, comprising the following steps 1) broth clarification, 2) ultrafiltration, 3) electrodialysis, 4) cation exchange, 5) concentration, 6) Activated Charcoal treatment, and 7) lyophilization.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) lyophilization.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) lyophilization.

Purification of a single oligosaccharide obtained from a cultivation or fermentation process, comprising the following steps 1) broth clarification through ultrafiltration, 4) cation exchange, 5) concentration, 6) Activated Charcoal treatment, and 7) lyophilization.

Purification of a single oligosaccharide obtained from a cultivation or fermentation process, comprising the following steps 1) broth clarification, 2) ultrafiltration, 3) cation exchange, 4) concentration, and 5) lyophilization.

Purification of a single oligosaccharide obtained from a cultivation or fermentation process, comprising the following steps 1) broth clarification through microfiltration, 2) ultrafiltration, 3) cation exchange, 4) concentration, and 5) lyophilization.

Purification of a single oligosaccharide obtained from a cultivation or fermentation process, comprising the following steps 1) broth clarification through centrifugation, 2) ultrafiltration, 3) cation exchange, 4) concentration, and 5) lyophilization.

Purification of a single oligosaccharide obtained from a cultivation or fermentation process, comprising the following steps 1) broth clarification through ultrafiltration, 2) cation exchange, 3) concentration, and 4) lyophilization.

Purification of a single oligosaccharide obtained from a cultivation or fermentation process, comprising the following steps 1) broth clarification, 2) ultrafiltration, 3) cation exchange, 4) concentration, 5) Activated Charcoal treatment, and 6) lyophilization.

Purification of a single oligosaccharide obtained from a cultivation or fermentation process, comprising the following steps 1) broth clarification through microfiltration, 2) ultrafiltration, 3) cation exchange, 4) concentration, 5) Activated Charcoal treatment, and 6) lyophilization.

Purification of a single oligosaccharide obtained from a cultivation or fermentation process, comprising the following steps 1) broth clarification through centrifugation, 2) ultrafiltration, 3) cation exchange, 4) concentration, 5) Activated Charcoal treatment, and 6) lyophilization.

Purification of a single oligosaccharide obtained from a cultivation or fermentation process, comprising the following steps 1) broth clarification through ultrafiltration, 2) cation exchange, 3) concentration, 4) Activated Charcoal treatment, and 5) lyophilization.

Purification of a single oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) lyophilization.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) lyophilization.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) lyophilization.

Purification of a single oligosaccharide obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) lyophilization.

Purification of a single oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) lyophilization.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) lyophilization.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) lyophilization.

Purification of a single oligosaccharide obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) lyophilization.

Purification of a single oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) lyophilization.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) lyophilization.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) lyophilization.

Purification of a single oligosaccharide obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) lyophilization.

Purification of a single oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) lyophilization.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) lyophilization.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) lyophilization.

Purification of a single oligosaccharide obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) lyophilization.

Purification of a single oligosaccharide obtained from a cultivation or fermentation process, comprising the following steps 1) broth clarification, 2) ultrafiltration, 3) cation exchange, 4) concentration, monosaccharide and disaccharide removal through nanofiltration, and 5) lyophilization.

Purification of a single oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) lyophilization.

Purification of a single oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) lyophilization.

Purification of a single oligosaccharide obtained from a cultivation or fermentation process, comprising the following steps 1) broth clarification through ultrafiltration, 2) cation exchange, 3) concentration, monosaccharide and disaccharide removal through nanofiltration, and 4) lyophilization.

Purification of a single oligosaccharide obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) lyophilization.

Purification of a single oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) lyophilization.

Purification of a single oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) lyophilization.

Purification of a single oligosaccharide obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) lyophilization.

Purification of a single oligosaccharide obtained from a cultivation or fermentation process, comprising the following steps 1) broth clarification, 2) ultrafiltration, 3) nanofiltration, 4) cation exchange, 5) concentration, and 6) spray drying.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, and 6) spray drying.

Purification of a single oligosaccharide obtained from a cultivation or centrifugation, 2) ultrafiltration, 3) nanofiltration, 4) cation exchange, 5) concentration, and 6) spray drying.

Purification of a single oligosaccharide obtained from a cultivation or fermentation process, comprising the following steps 1) broth clarification through ultrafiltration, 2) nanofiltration, 3) cation exchange, 4) concentration, and 5) spray drying.

Purification of a single oligosaccharide obtained from a cultivation or fermentation process, comprising the following steps 1) broth clarification, 2) ultrafiltration, 3) electrodialysis, 4) cation exchange, 5) concentration, and 6) spray drying.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, and 6) spray drying.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, and 6) spray drying.

Purification of a single oligosaccharide obtained from a cultivation or fermentation process, comprising the following steps 1) broth clarification through ultrafiltration, 2) electrodialysis, 3) cation exchange, 4) concentration, and 5) spray drying.

Purification of a single oligosaccharide obtained from a cultivation or fermentation process, comprising the following steps 1) broth clarification, 2) ultrafiltration, 3) nanofiltration, 4) cation exchange, 5) concentration, 6) Activated Charcoal treatment, and 7) spray drying.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) spray drying.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) spray drying.

Purification of a single oligosaccharide obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) spray drying.

Purification of a single oligosaccharide obtained from a cultivation or fermentation process, comprising the following steps 1) broth clarification, 2) ultrafiltration, 3) electrodialysis, 4) cation exchange, 5) concentration, 6) Activated Charcoal treatment, and 7) spray drying.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) spray drying.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) spray drying.

Purification of a single oligosaccharide obtained from a cultivation or fermentation process, comprising the following steps 1) broth clarification through ultrafiltration, 4) cation exchange, 5) concentration, 6) Activated Charcoal treatment, and 7) spray drying.

Purification of a single oligosaccharide obtained from a cultivation or fermentation process, comprising the following steps 1) broth clarification, 2) ultrafiltration, 3) cation exchange, 4) concentration, and 5) spray drying.

Purification of a single oligosaccharide obtained from a cultivation or fermentation process, comprising the following steps 1) broth clarification through microfiltration, 2) ultrafiltration, 3) cation exchange, 4) concentration, and 5) spray drying.

Purification of a single oligosaccharide obtained from a cultivation or fermentation process, comprising the following steps 1) broth clarification through centrifugation, 2) ultrafiltration, 3) cation exchange, 4) concentration, and 5) spray drying.

Purification of a single oligosaccharide obtained from a cultivation or fermentation process, comprising the following steps 1) broth clarification through ultrafiltration, 2) cation exchange, 3) concentration, and 4) spray drying.

Purification of a single oligosaccharide obtained from a cultivation or fermentation process, comprising the following steps 1) broth clarification, 2) ultrafiltration, 3) cation exchange, 4) concentration, 5) Activated Charcoal treatment, and 6) spray drying.

Purification of a single oligosaccharide obtained from a cultivation or fermentation process, comprising the following steps 1) broth clarification through microfiltration, 2) ultrafiltration, 3) cation exchange, 4) concentration, 5) Activated Charcoal treatment, and 6) spray drying.

Purification of a single oligosaccharide obtained from a cultivation or fermentation process, comprising the following steps 1) broth clarification through centrifugation, 2) ultrafiltration, 3) cation exchange, 4) concentration, 5) Activated Charcoal treatment, and 6) spray drying.

Purification of a single oligosaccharide obtained from a cultivation or fermentation process, comprising the following steps 1) broth clarification through ultrafiltration, 2) cation exchange, 3) concentration, 4) Activated Charcoal treatment, and 5) spray drying.

Purification of a single oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) spray drying.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) spray drying.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) spray drying.

Purification of a single oligosaccharide obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) spray drying.

Purification of a single oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) spray drying.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) spray drying.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) spray drying.

Purification of a single oligosaccharide obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) spray drying.

Purification of a single oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a single oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a single oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a single oligosaccharide obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) spray drying.

Purification of a single oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a single oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a single oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a single oligosaccharide obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) spray drying.

Purification of a single oligosaccharide obtained from a cultivation or fermentation process, comprising the following steps 1) broth clarification, 2) ultrafiltration, 3) cation exchange, 4) concentration, monosaccharide and disaccharide removal through nanofiltration, and 5) spray drying.

Purification of a single oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) spray drying.

Purification of a single oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) spray drying.

Purification of a single oligosaccharide obtained from a cultivation or fermentation process, comprising the following steps 1) broth clarification through ultrafiltration, 2) cation exchange, 3) concentration, monosaccharide and disaccharide removal through nanofiltration, and 4) spray drying.

Purification of a single oligosaccharide obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) spray drying.

Purification of a single oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) spray drying.

Purification of a single oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) spray drying.

Purification of a single oligosaccharide obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) spray drying.

Purification of a neutral oligosaccharide mixture obtained from a cultivation or fermentation process, comprising the following steps 1) broth clarification, 2) ultrafiltration, 3) nanofiltration, 4) cation exchange, 5) concentration, and 6) spray drying.

Purification of a neutral oligosaccharide mixture obtained from a cultivation or microfiltration, 2) ultrafiltration, 3) nanofiltration, 4) cation exchange, 5) concentration, and 6) spray drying.

Purification of a neutral oligosaccharide mixture obtained from a cultivation or fermentation process, comprising the following steps 1) broth clarification through centrifugation, 2) ultrafiltration, 3) nanofiltration, 4) cation exchange, 5) concentration, and 6) spray drying.

Purification of a neutral oligosaccharide mixture obtained from a cultivation or fermentation process, comprising the following steps 1) broth clarification through ultrafiltration, 2) nanofiltration, 3) cation exchange, 4) concentration, and 5) spray drying.

Purification of a neutral oligosaccharide mixture obtained from a cultivation or fermentation process, comprising the following steps 1) broth clarification, 2) ultrafiltration, 3) electrodialysis, 4) cation exchange, 5) concentration, and 6) spray drying.

Purification of a neutral oligosaccharide mixture obtained from a cultivation or fermentation process, comprising the following steps 1) broth clarification through microfiltration, 2) ultrafiltration, 3) electrodialysis, 4) cation exchange, 5) concentration, and 6) spray drying.

Purification of a neutral oligosaccharide mixture obtained from a cultivation or fermentation process, comprising the following steps 1) broth clarification through centrifugation, 2) ultrafiltration, 3) electrodialysis, 4) cation exchange, 5) concentration, and 6) spray drying.

Purification of a neutral oligosaccharide mixture obtained from a cultivation or fermentation process, comprising the following steps 1) broth clarification through ultrafiltration, 2) electrodialysis, 3) cation exchange, 4) concentration, and 5) spray drying.

Purification of a neutral oligosaccharide mixture obtained from a cultivation or fermentation process, comprising the following steps 1) broth clarification, 2) ultrafiltration, 3) nanofiltration, 4) cation exchange, 5) concentration, 6) Activated Charcoal treatment, and 7) spray drying.

Purification of a neutral oligosaccharide mixture obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) spray drying.

Purification of a neutral oligosaccharide mixture obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) spray drying.

Purification of a neutral oligosaccharide mixture obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) spray drying.

Purification of a neutral oligosaccharide mixture obtained from a cultivation or fermentation process, comprising the following steps 1) broth clarification, 2) ultrafiltration, 3) electrodialysis, 4) cation exchange, 5) concentration, 6) Activated Charcoal treatment, and 7) spray drying.

Purification of a neutral oligosaccharide mixture obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) spray drying.

Purification of a neutral oligosaccharide mixture obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) spray drying.

Purification of a neutral oligosaccharide mixture obtained from a cultivation or fermentation process, comprising the following steps 1) broth clarification through ultrafiltration, 4) cation exchange, 5) concentration, 6) Activated Charcoal treatment, and 7) spray drying.

Purification of a neutral oligosaccharide mixture obtained from a cultivation or fermentation process, comprising the following steps 1) broth clarification, 2) ultrafiltration, 3) cation exchange, 4) concentration, and 5) spray drying.

Purification of a neutral oligosaccharide mixture obtained from a cultivation or

Purification of a neutral oligosaccharide mixture obtained from a cultivation or fermentation process, comprising the following steps 1) broth clarification through centrifugation, 2) ultrafiltration, 3) cation exchange, 4) concentration, and 5) spray drying.

Purification of a neutral oligosaccharide mixture obtained from a cultivation or fermentation process, comprising the following steps 1) broth clarification through ultrafiltration, 2) cation exchange, 3) concentration, and 4) spray drying.

Purification of a neutral oligosaccharide mixture obtained from a cultivation or fermentation process, comprising the following steps 1) broth clarification, 2) ultrafiltration, 3) cation exchange, 4) concentration, 5) Activated Charcoal treatment, and 6) spray drying.

Purification of a neutral oligosaccharide mixture obtained from a cultivation or fermentation process, comprising the following steps 1) broth clarification through microfiltration, 2) ultrafiltration, 3) cation exchange, 4) concentration, 5) Activated Charcoal treatment, and 6) spray drying.

Purification of a neutral oligosaccharide mixture obtained from a cultivation or fermentation process, comprising the following steps 1) broth clarification through centrifugation, 2) ultrafiltration, 3) cation exchange, 4) concentration, 5) Activated Charcoal treatment, and 6) spray drying.

Purification of a neutral oligosaccharide mixture obtained from a cultivation or fermentation process, comprising the following steps 1) broth clarification through ultrafiltration, 2) cation exchange, 3) concentration, 4) Activated Charcoal treatment, and 5) spray drying.

Purification of a neutral oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) spray drying.

Purification of a neutral oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) spray drying.

Purification of a neutral oligosaccharide mixture obtained from a cultivation or centrifugation, 2) ultrafiltration, 3) nanofiltration, 4) cation exchange, 5) concentration, monosaccharide and disaccharide removal through nanofiltration, and 6) spray drying.

Purification of a neutral oligosaccharide mixture obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) spray drying.

Purification of a neutral oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) spray drying.

Purification of a neutral oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) spray drying.

Purification of a neutral oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) spray drying.

Purification of a neutral oligosaccharide mixture obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) spray drying.

Purification of a neutral oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a neutral oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a neutral oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a neutral oligosaccharide mixture obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) spray drying.

Purification of a neutral oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a neutral oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a neutral oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a neutral oligosaccharide mixture obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) spray drying.

Purification of a neutral oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, and 5) spray drying.

Purification of a neutral oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) spray drying.

Purification of a neutral oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) spray drying.

Purification of a neutral oligosaccharide mixture obtained from a cultivation or fermentation process, comprising the following steps 1) broth clarification through ultrafiltration, 2) cation exchange, 3) concentration, monosaccharide and disaccharide removal through nanofiltration, and 4) spray drying.

Purification of a neutral oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) spray drying.

Purification of a neutral oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) spray drying.

Purification of a neutral oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) spray drying.

Purification of a neutral oligosaccharide mixture obtained from a cultivation or fermentation process, comprising the following steps 1) broth clarification through ultrafiltration, 2) cation exchange, 3) concentration, monosaccharide and disaccharide removal through nonfiltration, 4) Activated Charcoal treatment, and 5) spray drying.

Purification of a neutral and acidic oligosaccharide mixture obtained from a cultivation or fermentation process, comprising the following steps 1) broth clarification, 2) ultrafiltration, 3) nanofiltration, 4) cation exchange, 5) concentration, and 6) spray drying.

Purification of a neutral and acid oligosaccharide mixture obtained from a cultivation or fermentation process, comprising the following steps 1) broth clarification through microfiltration, 2) ultrafiltration, 3) nanofiltration, 4) cation exchange, 5) concentration, and 6) spray drying.

Purification of a neutral and acid oligosaccharide mixture obtained from a cultivation or fermentation process, comprising the following steps 1) broth clarification through centrifugation, 2) ultrafiltration, 3) nanofiltration, 4) cation exchange, 5) concentration, and 6) spray drying.

Purification of a neutral and acid oligosaccharide mixture obtained from a cultivation or fermentation process, comprising the following steps 1) broth clarification through ultrafiltration, 2) nanofiltration, 3) cation exchange, 4) concentration, and 5) spray drying.

Purification of a neutral and acid oligosaccharide mixture obtained from a cultivation or fermentation process, comprising the following steps 1) broth clarification, 2) ultrafiltration, 3) electrodialysis, 4) cation exchange, 5) concentration, and 6) spray drying.

Purification of a neutral and acid oligosaccharide mixture obtained from a cultivation or fermentation process, comprising the following steps 1) broth clarification through microfiltration, 2) ultrafiltration, 3) electrodialysis, 4) cation exchange, 5) concentration, and 6) spray drying.

Purification of a neutral and acid oligosaccharide mixture obtained from a cultivation or fermentation process, comprising the following steps 1) broth clarification through centrifugation, 2) ultrafiltration, 3) electrodialysis, 4) cation exchange, 5) concentration, and 6) spray drying.

Purification of a neutral and acid oligosaccharide mixture obtained from a cultivation or fermentation process, comprising the following steps 1) broth clarification through ultrafiltration, 2) electrodialysis, 3) cation exchange, 4) concentration, and 5) spray drying.

Purification of a neutral and acid oligosaccharide mixture obtained from a cultivation or fermentation process, comprising the following steps 1) broth clarification, 2) ultrafiltration, 3) nanofiltration, 4) cation exchange, 5) concentration, 6) Activated Charcoal treatment, and 7) spray drying.

Purification of a neutral and acid oligosaccharide mixture obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) spray drying.

Purification of a neutral and acid oligosaccharide mixture obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) spray drying.

Purification of a neutral and acid oligosaccharide mixture obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) spray drying.

Purification of a neutral and acid oligosaccharide mixture obtained from a cultivation or fermentation process, comprising the following steps 1) broth clarification, 2) ultrafiltration, 3) electrodialysis, 4) cation exchange, 5) concentration, 6) Activated Charcoal treatment, and 7) spray drying.

Purification of a neutral and acid oligosaccharide mixture obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) spray drying.

Purification of a neutral and acid oligosaccharide mixture obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) spray drying.

Purification of a neutral and acid oligosaccharide mixture obtained from a cultivation or fermentation process, comprising the following steps 1) broth clarification through ultrafiltration, 4) cation exchange, 5) concentration, 6) Activated Charcoal treatment. and 7) spray drying.

Purification of a neutral and acid oligosaccharide mixture obtained from a cultivation or fermentation process, comprising the following steps 1) broth clarification, 2) ultrafiltration, 3) cation exchange, 4) concentration, and 5) spray drying.

Purification of a neutral and acid oligosaccharide mixture obtained from a cultivation or fermentation process, comprising the following steps 1) broth clarification through microfiltration, 2) ultrafiltration, 3) cation exchange, 4) concentration, and 5) spray drying.

Purification of a neutral and acid oligosaccharide mixture obtained from a cultivation or fermentation process, comprising the following steps 1) broth clarification through centrifugation, 2) ultrafiltration, 3) cation exchange, 4) concentration, and 5) spray drying.

Purification of a neutral and acid oligosaccharide mixture obtained from a cultivation or fermentation process, comprising the following steps 1) broth clarification through ultrafiltration, 2) cation exchange, 3) concentration, and 4) spray drying.

Purification of a neutral and acid oligosaccharide mixture obtained from a cultivation or fermentation process, comprising the following steps 1) broth clarification, 2) ultrafiltration, 3) cation exchange, 4) concentration, 5) Activated Charcoal treatment, and 6) spray drying.

Purification of a neutral and acid oligosaccharide mixture obtained from a cultivation or fermentation process, comprising the following steps 1) broth clarification through microfiltration, 2) ultrafiltration, 3) cation exchange, 4) concentration, 5) Activated Charcoal treatment. and 6) spray drying.

Purification of a neutral and acid oligosaccharide mixture obtained from a cultivation or fermentation process, comprising the following steps 1) broth clarification through centrifugation, 2) ultrafiltration, 3) cation exchange, 4) concentration, 5) Activated Charcoal treatment, and 6) spray drying.

Purification of a neutral and acid oligosaccharide mixture obtained from a cultivation or fermentation process, comprising the following steps 1) broth clarification through ultrafiltration, 2) cation exchange, 3) concentration, 4) Activated Charcoal treatment, and 5) spray drying.

Purification of a neutral and acid oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) spray drying.

Purification of a neutral and acid oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) spray drying.

Purification of a neutral and acid oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) spray drying.

Purification of a neutral and acid oligosaccharide mixture obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) spray drying.

Purification of a neutral and acid oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) spray drying.

Purification of a neutral and acid oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) spray drying.

Purification of a neutral and acid oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) spray drying.

Purification of a neutral and acid oligosaccharide mixture obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) spray drying.

Purification of a neutral and acid oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a neutral and acid oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a neutral and acid oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a neutral and acid oligosaccharide mixture obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) spray drying.

Purification of a neutral and acid oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a neutral and acid oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a neutral and acid oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a neutral and acid oligosaccharide mixture obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) spray drying.

Purification of a neutral and acid oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, and 5) spray drying.

Purification of a neutral and acid oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) spray drying.

Purification of a neutral and acid oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) spray drying.

Purification of a neutral and acid oligosaccharide mixture obtained from a cultivation or fermentation process, comprising the following steps 1) broth clarification through ultrafiltration, 2) cation exchange, 3) concentration, monosaccharide and disaccharide removal through nanofiltration, and 4) spray drying.

Purification of a neutral and acid oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) spray drying.

Purification of a neutral and acid oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) spray drying.

Purification of a neutral and acid oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) spray drying.

Purification of a neutral and acid oligosaccharide mixture obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) spray drying.

Purification of an acidic oligosaccharide mixture obtained from a cultivation or fermentation process, comprising the following steps 1) broth clarification, 2) ultrafiltration, 3) nanofiltration, 4) cation exchange, 5) concentration, and 6) spray drying.

Purification of an acidic oligosaccharide mixture obtained from a cultivation or fermentation process, comprising the following steps 1) broth clarification through microfiltration, 2) ultrafiltration, 3) nanofiltration, 4) cation exchange, 5) concentration, and 6) spray drying.

Purification of an acidic oligosaccharide mixture obtained from a cultivation or fermentation process, comprising the following steps 1) broth clarification through centrifugation, 2) ultrafiltration, 3) nanofiltration, 4) cation exchange, 5) concentration, and 6) spray drying.

Purification of an acidic oligosaccharide mixture obtained from a cultivation or fermentation process, comprising the following steps 1) broth clarification through ultrafiltration, 2) nanofiltration, 3) cation exchange, 4) concentration, and 5) spray drying.

Purification of an acidic oligosaccharide mixture obtained from a cultivation or fermentation process, comprising the following steps 1) broth clarification, 2) ultrafiltration, 3) electrodialysis, 4) cation exchange, 5) concentration, and 6) spray drying.

Purification of an acidic oligosaccharide mixture obtained from a cultivation or fermentation process, comprising the following steps 1) broth clarification through microfiltration, 2) ultrafiltration, 3) electrodialysis, 4) cation exchange, 5) concentration, and 6) spray drying.

Purification of an acidic oligosaccharide mixture obtained from a cultivation or fermentation process, comprising the following steps 1) broth clarification through centrifugation, 2) ultrafiltration, 3) electrodialysis, 4) cation exchange, 5) concentration, and 6) spray drying.

Purification of an acidic oligosaccharide mixture obtained from a cultivation or fermentation process, comprising the following steps 1) broth clarification through ultrafiltration, 2) electrodialysis, 3) cation exchange, 4) concentration, and 5) spray drying.

Purification of an acidic oligosaccharide mixture obtained from a cultivation or fermentation process, comprising the following steps 1) broth clarification, 2) ultrafiltration, 3) nanofiltration, 4) cation exchange, 5) concentration, 6) Activated Charcoal treatment, and 7) spray drying.

Purification of an acidic oligosaccharide mixture obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) spray drying.

Purification of an acidic oligosaccharide mixture obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) spray drying.

Purification of an acidic oligosaccharide mixture obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) spray drying.

Purification of an acidic oligosaccharide mixture obtained from a cultivation or fermentation process, comprising the following steps 1) broth clarification, 2) ultrafiltration, 3) electrodialysis, 4) cation exchange, 5) concentration, 6) Activated Charcoal treatment, and 7) spray drying.

Purification of an acidic oligosaccharide mixture obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) spray drying.

Purification of an acidic oligosaccharide mixture obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) spray drying.

Purification of an acidic oligosaccharide mixture obtained from a cultivation or fermentation process, comprising the following steps 1) broth clarification through ultrafiltration, 4) cation exchange, 5) concentration, 6) Activated Charcoal treatment 7) spray drying.

Purification of an acidic oligosaccharide mixture obtained from a cultivation or fermentation process, comprising the following steps 1) broth clarification, 2) ultrafiltration, 3) cation exchange, 4) concentration, and 5) spray drying.

Purification of an acidic oligosaccharide mixture obtained from a cultivation or fermentation process, comprising the following steps 1) broth clarification through microfiltration, 2) ultrafiltration, 3) cation exchange, 4) concentration, and 5) spray drying.

Purification of an acidic oligosaccharide mixture obtained from a cultivation or fermentation process, comprising the following steps 1) broth clarification through centrifugation, 2) ultrafiltration, 3) cation exchange, 4) concentration, and 5) spray drying.

Purification of an acidic oligosaccharide mixture obtained from a cultivation or fermentation process, comprising the following steps 1) broth clarification through ultrafiltration, 2) cation exchange, 3) concentration, and 4) spray drying.

Purification of an acidic oligosaccharide mixture obtained from a cultivation or fermentation process, comprising the following steps 1) broth clarification, 2) ultrafiltration, 3) cation exchange, 4) concentration, 5) Activated Charcoal treatment, and 6) spray drying.

Purification of an acidic oligosaccharide mixture obtained from a cultivation or fermentation process, comprising the following steps 1) broth clarification through microfiltration, 2) ultrafiltration, 3) cation exchange, 4) concentration, 5) Activated Charcoal treatment, and 6) spray drying.

Purification of an acidic oligosaccharide mixture obtained from a cultivation or fermentation process, comprising the following steps 1) broth clarification through centrifugation, 2) ultrafiltration, 3) cation exchange, 4) concentration, 5) Activated Charcoal treatment, and 6) spray drying.

Purification of an acidic oligosaccharide mixture obtained from a cultivation or fermentation process, comprising the following steps 1) broth clarification through ultrafiltration, 2) cation exchange, 3) concentration, 4) Activated Charcoal treatment, and 5) spray drying.

Purification of an acidic oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) spray drying.

Purification of an acidic oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) spray drying.

Purification of an acidic oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) spray drying.

Purification of an acidic oligosaccharide mixture obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) spray drying.

Purification of an acidic oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) spray drying.

Purification of an acidic oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) spray drying.

Purification of an acidic oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) spray drying.

Purification of an acidic oligosaccharide mixture obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) spray drying.

Purification of an acidic oligosaccharide mixture obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) spray drying.

Purification of an acidic oligosaccharide mixture obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) spray drying.

Purification of an acidic oligosaccharide mixture obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) spray drying.

Purification of an acidic oligosaccharide mixture obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) spray drying.

Purification of an acidic oligosaccharide mixture obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) spray drying.

Purification of an acidic oligosaccharide mixture obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) spray drying.

Purification of an acidic oligosaccharide mixture obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) spray drying.

Purification of an acidic oligosaccharide mixture obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) spray drying.

Purification of an acidic oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, and 5) spray drying.

Purification of an acidic oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) spray drying.

Purification of an acidic oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) spray drying.

Purification of an acidic oligosaccharide mixture obtained from a cultivation or fermentation process, comprising the following steps 1) broth clarification through ultrafiltration, 2) cation exchange, 3) concentration, monosaccharide and disaccharide removal through nanofiltration, and 4) spray drying.

Purification of an acidic oligosaccharide mixture obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) spray drying.

Purification of an acidic oligosaccharide mixture obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) spray drying.

Purification of an acidic oligosaccharide mixture obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) spray drying.

Purification of an acidic oligosaccharide mixture obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) spray drying.

Purification of a single acidic oligosaccharide obtained from a cultivation or fermentation process, comprising the following steps 1) broth clarification, 2) ultrafiltration, 3) nanofiltration, 4) cation exchange, 5) concentration, and 6) crystallization.

Purification of a single acidic oligosaccharide obtained from a cultivation or fermentation process, comprising the following steps 1) broth clarification through microfiltration, 2) ultrafiltration, 3) nanofiltration, 4) cation exchange, 5) concentration, and 6) crystallization.

Purification of a single acidic oligosaccharide obtained from a cultivation or fermentation process, comprising the following steps 1) broth clarification through centrifugation, 2) ultrafiltration, 3) nanofiltration, 4) cation exchange, 5) concentration, and 6) crystallization.

Purification of a single acidic oligosaccharide obtained from a cultivation or fermentation process, comprising the following steps 1) broth clarification through ultrafiltration, 2) nanofiltration, 3) cation exchange, 4) concentration, and 5) crystallization.

Purification of a single acidic oligosaccharide obtained from a cultivation or fermentation process, comprising the following steps 1) broth clarification, 2) ultrafiltration, 3) electrodialysis, 4) cation exchange, 5) concentration, and 6) crystallization.

Purification of a single acidic oligosaccharide obtained from a cultivation or fermentation process, comprising the following steps 1) broth clarification through microfiltration, 2) ultrafiltration, 3) electrodialysis, 4) cation exchange, 5) concentration, and 6) crystallization.

Purification of a single acidic oligosaccharide obtained from a cultivation or fermentation process, comprising the following steps 1) broth clarification through centrifugation, 2) ultrafiltration, 3) electrodialysis, 4) cation exchange, 5) concentration, and 6) crystallization.

Purification of a single acidic oligosaccharide obtained from a cultivation or fermentation process, comprising the following steps 1) broth clarification through ultrafiltration, 2) electrodialysis, 3) cation exchange, 4) concentration, and 5) crystallization.

Purification of a single acidic oligosaccharide obtained from a cultivation or fermentation process, comprising the following steps 1) broth clarification, 2) ultrafiltration, 3) nanofiltration, 4) cation exchange, 5) concentration, 6) Activated Charcoal treatment, and 7) crystallization.

Purification of a single acidic oligosaccharide obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) crystallization.

Purification of a single acidic oligosaccharide obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) crystallization.

Purification of a single acidic oligosaccharide obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) crystallization.

Purification of a single acidic oligosaccharide obtained from a cultivation or fermentation process, comprising the following steps 1) broth clarification, 2) ultrafiltration, 3) electrodialysis, 4) cation exchange, 5) concentration, 6) Activated Charcoal treatment, and 7) crystallization.

Purification of a single acidic oligosaccharide obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) crystallization.

Purification of a single acidic oligosaccharide obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) crystallization.

Purification of a single acidic oligosaccharide obtained from a cultivation or fermentation process, comprising the following steps 1) broth clarification through ultrafiltration, 4) cation exchange, 5) concentration, 6) Activated Charcoal treatment, and 7) crystallization.

Purification of a single acidic oligosaccharide obtained from a cultivation or fermentation process, comprising the following steps 1) broth clarification, 2) ultrafiltration, 3) cation exchange, 4) concentration, and 5) crystallization.

Purification of a single acidic oligosaccharide obtained from a cultivation or fermentation process, comprising the following steps 1) broth clarification through microfiltration, 2) ultrafiltration, 3) cation exchange, 4) concentration, and 5) crystallization.

Purification of a single acidic oligosaccharide obtained from a cultivation or fermentation process, comprising the following steps 1) broth clarification through centrifugation, 2) ultrafiltration, 3) cation exchange, 4) concentration, and 5) crystallization.

Purification of a single acidic oligosaccharide obtained from a cultivation or fermentation process, comprising the following steps 1) broth clarification through ultrafiltration, 2) cation exchange, 3) concentration, and 4) crystallization.

Purification of a single acidic oligosaccharide obtained from a cultivation or fermentation process, comprising the following steps 1) broth clarification, 2) ultrafiltration, 3) cation exchange, 4) concentration, 5) Activated Charcoal treatment, and 6) crystallization.

Purification of a single acidic oligosaccharide obtained from a cultivation or fermentation process, comprising the following steps 1) broth clarification through microfiltration, 2) ultrafiltration, 3) cation exchange, 4) concentration, 5) Activated Charcoal treatment, and 6) crystallization.

Purification of a single acidic oligosaccharide obtained from a cultivation or fermentation process, comprising the following steps 1) broth clarification through centrifugation, 2) ultrafiltration, 3) cation exchange, 4) concentration, 5) Activated Charcoal treatment, and 6) crystallization.

Purification of a single acidic oligosaccharide obtained from a cultivation or fermentation process, comprising the following steps 1) broth clarification through ultrafiltration, 2) cation exchange, 3) concentration, 4) Activated Charcoal treatment, and 5) crystallization.

Purification of a single acidic oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) crystallization.

Purification of a single acidic oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) crystallization.

Purification of a single acidic oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, and 6) crystallization.

Purification of a single acidic oligosaccharide obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) crystallization.

Purification of a single acidic oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) crystallization.

Purification of a single acidic oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) crystallization.

Purification of a single acidic oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) crystallization.

Purification of a single acidic oligosaccharide obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) crystallization.

Purification of a single acidic oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) crystallization.

Purification of a single acidic oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) crystallization.

Purification of a single acidic oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) crystallization.

Purification of a single acidic oligosaccharide obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) crystallization.

Purification of a single acidic oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) crystallization.

Purification of a single acidic oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) crystallization.

Purification of a single acidic oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) crystallization.

Purification of a single acidic oligosaccharide obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) crystallization.

Purification of a single acidic oligosaccharide obtained from a cultivation or fermentation process, comprising the following steps 1) broth clarification, 2) ultrafiltration, 3) cation exchange, 4) concentration, monosaccharide and disaccharide removal through nanofiltration, and 5) crystallization.

Purification of a single acidic oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) crystallization.

Purification of a single acidic oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) crystallization.

Purification of a single acidic oligosaccharide obtained from a cultivation or fermentation process, comprising the following steps 1) broth clarification through ultrafiltration, 2) cation exchange, 3) concentration, monosaccharide and disaccharide removal through nanofiltration, and 4) crystallization.

Purification of a single acidic oligosaccharide obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) crystallization.

Purification of a single acidic oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment, and 6) crystallization.

Purification of a single acidic oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) crystallization.

Purification of a single acidic oligosaccharide obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) crystallization.

Purification of a single acidic oligosaccharide obtained from a cultivation or fermentation process, comprising the following steps 1) broth clarification, 2) ultrafiltration, 3) nanofiltration, 4) cation exchange, 5) concentration, and 6) spray drying.

Purification of a single acidic oligosaccharide obtained from a cultivation or microfiltration, 2) ultrafiltration, 3) nanofiltration, 4) cation exchange, 5) concentration, and 6) spray drying.

Purification of a single acidic oligosaccharide obtained from a cultivation or fermentation process, comprising the following steps 1) broth clarification through centrifugation, 2) ultrafiltration, 3) nanofiltration, 4) cation exchange, 5) concentration, and 6) spray drying.

Purification of a single acidic oligosaccharide obtained from a cultivation or fermentation process, comprising the following steps 1) broth clarification through ultrafiltration, 2) nanofiltration, 3) cation exchange, 4) concentration, 5) spray drying.

Purification of a single acidic oligosaccharide obtained from a cultivation or fermentation process, comprising the following steps 1) broth clarification, 2) ultrafiltration, 3) electrodialysis, 4) cation exchange, 5) concentration, and 6) spray drying.

Purification of a single acidic oligosaccharide obtained from a cultivation or fermentation process, comprising the following steps 1) broth clarification through microfiltration, 2) ultrafiltration, 3) electrodialysis, 4) cation exchange, 5) concentration, and 6) spray drying.

Purification of a single acidic oligosaccharide obtained from a cultivation or fermentation process, comprising the following steps 1) broth clarification through centrifugation, 2) ultrafiltration, 3) electrodialysis, 4) cation exchange, 5) concentration, and 6) spray drying.

Purification of a single acidic oligosaccharide obtained from a cultivation or fermentation process, comprising the following steps 1) broth clarification through ultrafiltration, 2) electrodialysis, 3) cation exchange, 4) concentration, and 5) spray drying.

Purification of a single acidic oligosaccharide obtained from a cultivation or fermentation process, comprising the following steps 1) broth clarification, 2) ultrafiltration, 3) nanofiltration, 4) cation exchange, 5) concentration, 6) Activated Charcoal treatment, and 7) spray drying.

Purification of a single acidic oligosaccharide obtained from a cultivation or fermentation process, comprising the following steps 1) broth clarification through microfiltration, 2) ultrafiltration, 3) nanofiltration, 4) cation exchange, 5) concentration, 6) Activated Charcoal treatment, and 7) spray drying.

Purification of a single acidic oligosaccharide obtained from a cultivation or fermentation process, comprising the following steps 1) broth clarification through centrifugation, 2) ultrafiltration, 3) nanofiltration, 4) cation exchange, 5) concentration, 6) Activated Charcoal treatment, and 7) spray drying.

Purification of a single acidic oligosaccharide obtained from a cultivation or fermentation process, comprising the following steps 1) broth clarification through ultrafiltration, 2) nanofiltration, 3) cation exchange, 4) cation exchange, 5) concentration, 6) Activated Charcoal treatment, and 7) spray drying.

Purification of a single acidic oligosaccharide obtained from a cultivation or fermentation process, comprising the following steps 1) broth clarification, 2) ultrafiltration, 3) electrodialysis, 4) cation exchange, 5) concentration, 6) Activated Charcoal treatment 7) spray drying.

Purification of a single acidic oligosaccharide obtained from a cultivation or fermentation process, comprising the following steps 1) broth clarification through microfiltration, 2) ultrafiltration, 3) electrodialysis, 4) cation exchange, 5) concentration, 6) Activated Charcoal treatment, and 7) spray drying.

Purification of a single acidic oligosaccharide obtained from a cultivation or fermentation process, comprising the following steps 1) broth clarification through centrifugation, 2) ultrafiltration, 3) electrodialysis, 4) cation exchange, 5) concentration, 6) Activated Charcoal treatment, and 7) spray drying.

Purification of a single acidic oligosaccharide obtained from a cultivation or fermentation process, comprising the following steps 1) broth clarification through ultrafiltration, 4) cation exchange, 5) concentration, 6) Activated Charcoal treatment, and 7) spray drying.

Purification of a single acidic oligosaccharide obtained from a cultivation or fermentation process, comprising the following steps 1) broth clarification, 2) ultrafiltration, 3) cation exchange, 4) concentration, and 5) spray drying.

Purification of a single acidic oligosaccharide obtained from a cultivation or

Purification of a single acidic oligosaccharide obtained from a cultivation or fermentation process, comprising the following steps 1) broth clarification through centrifugation, 2) ultrafiltration, 3) cation exchange, 4) concentration, and 5) spray drying.

Purification of a single acidic oligosaccharide obtained from a cultivation or fermentation process, comprising the following steps 1) broth clarification through ultrafiltration, 2) cation exchange, 3) concentration, and 4) spray drying.

Purification of a single acidic oligosaccharide obtained from a cultivation or fermentation process, comprising the following steps 1) broth clarification, 2) ultrafiltration, 3) cation exchange, 4) concentration, 5) Activated Charcoal treatment, and 6) spray drying.

Purification of a single acidic oligosaccharide obtained from a cultivation or fermentation process, comprising the following steps 1) broth clarification through microfiltration, 2) ultrafiltration, 3) cation exchange, 4) concentration, 5) Activated Charcoal treatment, and 6) spray drying.

Purification of a single acidic oligosaccharide obtained from a cultivation or fermentation process, comprising the following steps 1) broth clarification through centrifugation, 2) ultrafiltration, 3) cation exchange, 4) concentration, 5) Activated Charcoal treatment, and 6) spray drying.

Purification of a single acidic oligosaccharide obtained from a cultivation or fermentation process, comprising the following steps 1) broth clarification through ultrafiltration, 2) cation exchange, 3) concentration, 4) Activated Charcoal treatment, and 5) spray drying.

Purification of a single acidic oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, and 6) spray drying.

Purification of a single acidic oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, and 6) spray drying.

Purification of a single acidic oligosaccharide obtained from a cultivation or centrifugation, 2) ultrafiltration, 3) nanofiltration, 4) cation exchange, 5) concentration, monosaccharide and disaccharide removal through nanofiltration, and 6) spray drying.

Purification of a single acidic oligosaccharide obtained from a cultivation or fermentation process, comprising the following steps 1) broth clarification through ultrafiltration, 2) nanofiltration, 3) cation exchange, 4) concentration, monosaccharide and disaccharide removal through nanofiltration, and 5) spray drying.

Purification of a single acidic oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, and 6) spray drying.

Purification of a single acidic oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, and 6) spray drying.

Purification of a single acidic oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, and 6) spray drying.

Purification of a single acidic oligosaccharide obtained from a cultivation or fermentation process, comprising the following steps 1) broth clarification through ultrafiltration, 2) electrodialysis, 3) cation exchange, 4) concentration, monosaccharide and disaccharide removal through nanofiltration, and 5) spray drying.

Purification of a single acidic oligosaccharide obtained from a cultivation or fermentation process, comprising the following steps 1) broth clarification, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a single acidic oligosaccharide obtained from a cultivation or fermentation process, comprising the following steps 1) broth clarification through microfiltration, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a single acidic oligosaccharide obtained from a cultivation or fermentation process, comprising the following steps 1) broth clarification through centrifugation, 2) ultrafiltration, 3) nanofiltration, 4) cation exchange, 5) concentration, monosaccharide and disaccharide removal through nanofiltration, 6) Activated Charcoal treatment, and 7) spray drying.

Purification of a single acidic oligosaccharide obtained from a cultivation or fermentation process, comprising the following steps 1) broth clarification through ultrafiltration, 2) nanofiltration, 3) cation exchange, 4) cation exchange, 5) concentration, monosaccharide and disaccharide removal through nanofiltration, 6) Activated Charcoal treatment, and 7) spray drying.

Purification of a single acidic oligosaccharide obtained from a cultivation or fermentation process, comprising the following steps 1) broth clarification, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a single acidic oligosaccharide obtained from a cultivation or fermentation process, comprising the following steps 1) broth clarification through microfiltration, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a single acidic oligosaccharide obtained from a cultivation or fermentation process, comprising the following steps 1) broth clarification through centrifugation, 2) ultrafiltration, 3) electrodialysis, 4) cation exchange, 5) concentration, monosaccharide and disaccharide removal through nanofiltration, 6) Activated Charcoal treatment, and 7) spray drying.

Purification of a single acidic oligosaccharide obtained from a cultivation or fermentation process, comprising the following steps 1) broth clarification through ultrafiltration, 4) cation exchange, 5) concentration, monosaccharide and disaccharide removal through nanofiltration, 6) Activated Charcoal treatment, and 7) spray drying.

Purification of a single acidic oligosaccharide obtained from a cultivation or fermentation process, comprising the following steps 1) broth clarification, 2) ultrafiltration, 3) cation exchange, 4) concentration, monosaccharide and disaccharide removal through nanofiltration, and 5) spray drying.

Purification of a single acidic oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, and 5) spray drying.

Purification of a single acidic oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, and 5) spray drying.

Purification of a single acidic oligosaccharide obtained from a cultivation or fermentation process, comprising the following steps 1) broth clarification through ultrafiltration, 2) cation exchange, 3) concentration, monosaccharide and disaccharide removal through nanofiltration, and 4) spray drying.

Purification of a single acidic oligosaccharide obtained from a cultivation or fermentation process, comprising the following steps 1) broth clarification, 2) ultrafiltration, 3) cation exchange, 4) concentration, monosaccharide and disaccharide removal through nanofiltration, 5) Activated Charcoal treatment, and 6) spray drying.

Purification of a single acidic oligosaccharide obtained from a cultivation or fermentation process, comprising the following steps 1) broth clarification through microfiltration, 2) ultrafiltration, 3) cation exchange, 4) concentration, monosaccharide and disaccharide removal through nanofiltration, 5) Activated Charcoal treatment. and 6) spray drying.

Purification of a single acidic oligosaccharide obtained from a cultivation or fermentation process, comprising the following steps 1) broth clarification through centrifugation, 2) ultrafiltration, 3) cation exchange, 4) concentration, monosaccharide and disaccharide removal through nanofiltration, 5) Activated Charcoal treatment, and 6) spray drying.

Purification of a single acidic oligosaccharide obtained from a cultivation or fermentation process, comprising the following steps 1) broth clarification through ultrafiltration, 2) cation exchange, 3) concentration, monosaccharide and disaccharide removal through nanofiltration, 4) Activated Charcoal treatment, and 5) spray drying.

1.-83. (canceled)

84. A process for purifying an oligosaccharide solution in a batch or continuous manner from a cultivation or fermentation broth obtained by cell cultivation or microbial fermentation, wherein the cultivation or fermentation broth further comprises biomass, medium components and contaminants, optionally wherein the oligosaccharide solution's purity in the cultivation or fermentation broth is <80% on total dry solid, and wherein the cultivation or fermentation broth is applied to the following purification steps:
i) clarifying the cultivation or fermentation broth,
ii) removing salts and/or medium components from the clarified cultivation or fermentation broth, and
iii) optionally concentrating the oligosaccharide solution,
wherein a purified oligosaccharide solution at a purity of ≥80% on total dry solid is provided,
wherein step ii) of removing salts and/or medium components from the clarified cultivation or fermentation broth comprises an ion exchanger treatment consisting of cationic ion exchanger treatment for removing positively charged material and which does not comprise an anionic ion exchange treatment for removing negatively charged material.

85. The process of claim 84, wherein step iii) comes before step ii).

86. The process of claim 84, wherein the purified oligosaccharide solution has an ash content of ≤10% on total dry solid.

87. The process of claim 84, wherein the oligosaccharide solution is purified from a cultivation or fermentation broth obtained by cell cultivation, using at least one cell, wherein biomass separated in step i) is optionally recycled to the cultivation.

88. The process of claim 84, wherein the oligosaccharide solution is purified from a fermentation broth obtained by microbial fermentation using at least one micro-organism, wherein biomass separated in step i) is optionally recycled to the microbial fermentation.

89. The process of claim 84, wherein the cell cultivation utilizes a recombinant cell and comprises at least one cell that has been genetically modified to produce oligosaccharide.

90. The process of claim 84, wherein the cell cultivation is a microbial fermentation using a recombinant micro-organism and comprises at least one micro-organism that has been genetically modified to produce oligosaccharide.

91. The process of claim 84, wherein the oligosaccharide solution in the cultivation or fermentation broth is obtained by cell cultivation or microbial fermentation using at least one genetically modified cell capable of producing the oligosaccharide solution.

92. The process of claim 84, wherein the cell cultivation or microbial fermentation is cultured in a minimal salt medium with a carbon source on which the at least one cell or micro-organism grows, optionally
the minimal salt medium contains sulphate, phosphate, chloride, ammonium, calcium ion, magnesium ion, sodium, potassium ion, iron ion, copper ion, zinc ion, manganese ion, cobalt ion, and/or selenium ion, and
the carbon source comprises one or more of glucose, fructose, mannose, sucrose, maltose, corn steep liquor, lactose, galactose, high fructose syrup, starch, cellulose, hemi-cellulose, malto-oligosaccharides, trehalose, glycerol, acetate, citrate, lactate and pyruvate.

93. The process of claim 84, wherein the purity of the oligosaccharide solution in the cultivation or fermentation broth is <70%, <60%, <50%, <40%, <30%, <20%, <10% on total dry solid, before the purification and/or the purity of the purified oligosaccharide solution is >80% on total dry solid after the purification.

94. The process of claim 84, wherein the yield of purification of the oligosaccharide solution is >60%.

95. The process of claim 84, wherein the oligosaccharide solution comprises at least 2 different oligosaccharides.

96. The process of claim 95, wherein the oligosaccharide solution comprises at least 2 different oligosaccharides which differ in degree of polymerization.

97. The process of claim 95, wherein the oligosaccharide solution comprises at least one neutral and at least one charged oligosaccharide.

98. The process of claim 84, wherein the step i) of clarifying the cultivation or fermentation broth comprises one or more of clarification, clearing, filtration, microfiltration, centrifugation, decantation and ultrafiltration.

99. The process of claim 84, wherein the step ii) of removing salts and/or medium components from the clarified cultivation or fermentation broth further comprises at least one or more of nanofiltration, dialysis, electrodialysis, use of activated charcoal or carbon, use of solvents, use of alcohols, and use of aqueous alcohol mixtures, use of charcoal, tangential flow high-performance filtration, tangential flow ultrafiltration, affinity chromatography, cation exchange, simulated moving bed chromatography, hydrophobic interaction chromatography, gel filtration, ligand exchange chromatography, column chromatography, cation exchange adsorbent resin, and use of cation exchange resin.

100. The process of claim 84, wherein the step iii) of concentrating comprises one or more of nanofiltration, diafiltration, reverse osmosis, evaporation, wiped film evaporation, and falling film evaporation.

101. The process of claim 84, wherein the oligosaccharide solution comprises at least one oligosaccharide selected from the group consisting of fucosylated oligosaccharide, sialylated oligosaccharide, Lewis type antigen, N-acetylglucosamine containing neutral oligosaccharide, N-acetyllactosamine containing oligosaccharide, lacto-N-biose containing oligosaccharide, non-fucosylated neutral oligosaccharide, chitosan, chitosan oligosaccharide, heparosan, chondroitin sulphate, glycosaminoglycan oligosaccharide, heparin, heparan sulphate, chondroitin sulphate, dermatan sulphate, hyaluronan acid, r hyaluronic acid, and keratan sulphate.

102. The process of claim 84, wherein the step i) comprises a first step of clarification by microfiltration, centrifugation, flocculation or ultrafiltration.

103. The process of claim 84, wherein the step i) comprises ultrafiltration.

104. The process of claim 84, wherein step i) comprises two consecutive ultrafiltrations, and wherein the membrane molecular weight cut-off of the first ultrafiltration is higher than that of the second ultrafiltration.

105. The process of claim 84, wherein step ii) comprises nanofiltration and/or electrodialysis.

106. The process of claim 23, wherein the ultrafiltration permeate of step i) is nanofiltered and/or electrodialysed in step ii).

107. The process of claim 84, wherein step i) is ultrafiltration, step ii) is nanofiltration and/or electrodialysis treatment combined with a cation exchange treatment.

108. The process of claim 105, wherein the molecular weight cut-off of the nanofiltration membrane in step ii) is lower than that of the ultrafiltration membrane in step i).

109. The process of claim 84, wherein the cationic ion exchanger treatment is a strongly acidic cation exchanger treatment.

110. The process of claim 84, wherein the pH of the eluent of the cation exchange treatment is controlled to keep pH between 4 and 7.

111. The process of claim 84, wherein step ii) comprises electrodialysis.

112. The process of claim 84, wherein at least one of the purification steps i) to iii) is repeated at least one time during the process.

113. The process of claim 84, wherein after at least one of the purification steps i) or ii); the oligosaccharide solution is diafiltered and/or concentrated.

114. The process of claim 84, wherein the purified oligosaccharide solution has a Brix value of from about 8 to about 75%.

115. The process of claim 84, wherein the purified oligosaccharide solution is sterile filtered and/or subjected to endotoxin removal.

116. The process of claim 84, wherein step i) is preceded by an enzymatic treatment, optionally the enzymatic treatment comprises incubation of the broth with one or more enzymes selected from the group consisting of glycosidase, lactase, b-galactosidase, fucosidase, sialidase, maltase, amylase, hexaminidase, glucuronidase, trehalase, and invertase, optionally the enzymatic treatment converts lactose and/or sucrose to monosaccharides.

117. The process of claim 84, wherein the method further comprises decolorization.

118. The process of claim 84, wherein the purified oligosaccharide solution has an ash content below 10% (on total dry solid).

119. The process of claim 84, wherein the purified oligosaccharide solution has a protein content below 100 mg per kg dry solid, DNA content below 10 ng per gram dry solid and/or endotoxin content below 10000 EU per gram dry solid.

120. The process of claim 84, wherein the cell cultivation or microbial fermentation comprises at least one cell, wherein the at least one cell is a cell of a bacterium, a fungus, a yeast, a plant, animal, or protozoan cell.

121. The process of claim 84, wherein the cell cultivation or microbial fermentation comprises at least one cell, wherein the at least one cell is the cell of a micro-organism, wherein the micro-organism is an E. coli or yeast of lactose permease positive phenotype wherein the lactose permease is coded by the gene LacY or LAC12, respectively.

122. The process of claim 84, wherein the purified oligosaccharide solution is further concentrated to a syrup of at least 40% dry matter, the purified oligosaccharide solution is crystallised or the purified oligosaccharide solution is dried to a powder.

123. The process of claim 84, wherein step iii) comprises using vacuum evaporation or reverse osmosis or nanofiltration a) to an oligosaccharide concentration of >100 g/L and/or b) at a temperature of <60° C., during vacuum evaporation or reverse osmosis, and/or c) at a temperature of <80° C.

124. The process of claim 84, wherein the purified oligosaccharide solution comprises one oligosaccharide and is concentrated to a concentration of >1.5 M and cooled to a temperature <25° C. to obtain crystalline material of the oligosaccharide.

125. The process of claim 84, wherein the purified oligosaccharide solution is dried, and optionally the step of drying comprises any one or more of spray drying, lyophilization, evaporation, precipitation, spray freeze drying, freeze spray drying, band drying, belt drying, vacuum band drying, vacuum belt drying, drum drying, vacuum drum drying, roller drying, vacuum roller drying and other types of drying.

126. The process of claim 125, wherein the purified oligosaccharide solution is spray-dried.

127. The process of claim 125, wherein the drying is spray-drying or freeze-drying the purified oligosaccharide solution.

128. The process of claim 125, wherein the purified solution is spray-dried.

129. A purified oligosaccharide solution, oligosaccharide, or oligosaccharide mixture produced by the process of claim 84.

130. An oligosaccharide produced by the process of claim 84, wherein the oligosaccharide solution is spray-dried, lyophilized or crystallized.

131. An oligosaccharide mixture produced by the process of claim 84, wherein the oligosaccharide solution is spray-dried, lyophilized or concentrated to a syrup of at least 40% dry matter.

132. An oligosaccharide solution, spray dried oligosaccharide or oligosaccharide mixture produced through cultivation or fermentation, wherein the oligosaccharide solution is purified without anion exchange and wherein the oligosaccharide solution contains less than 10% ash.

133. The spray dried oligosaccharide or oligosaccharide mixture of claim 132, wherein the oligosaccharide or oligosaccharide mixture is purified without anion exchange and wherein the spray dried oligosaccharide or oligosaccharide mixture contains less than 10% ash.

134. Dried powder produced by the process of claim 125, wherein the dried powder contains ≤15%-wt. of water.

135. Spray dried powder produced by the process of claim 126, wherein the spray dried powder has a mean particle size of 50 to 250 μm as determined by laser diffraction.

136. Dried powder produced by the process of claim 125, wherein the powder exhibits:
a loose bulk density of from about 500 to 700 g/L,
a 100× tapped bulk density of from about 600 to about 850 g/L
a 625× tapped bulk density of from about 600 to about 900 g/L, and/or
a 1250× tapped bulk density of from about 650 to about 900 g/L.

137. The dried powder of claim 136, wherein the powder exhibits:
i)
a loose bulk density of from about 600 to 700 g/L,
a 100× tapped bulk density of from about 750 to about 850 g/L
a 625× tapped bulk density of from about 750 to about 850 g/L, and/or
a 1250× tapped bulk density of from about 850 to about 900 g/L, or
ii)
a loose bulk density of from about 500 to 600 g/L,
a 100× tapped bulk density of from about 600 to about 700 g/L
a 625× tapped bulk density of from about 700 to about 800 g/L, and/or
a 1250× tapped bulk density of from about 750 to about 800 g/L.

138. The dried powder of claim 130, wherein the powder when re-dissolved in water at a concentration of 10% (mass on volume) provides a solution with a pH between 4 and 7.

139. The oligosaccharide or oligosaccharide mixture of claim 129, wherein the oligosaccharide or oligosaccharide mixture a) has a conductivity of less than 1 mS/cm at a 300 g/l solution; b) is free of recombinant DNA material, optionally free of any DNA; and/or c) is free of proteins derived from the recombinant micro-organism, optionally free of any proteins.

140. A medicine comprising: the oligosaccharide or oligosaccharide mixture of claim 129.

141. The method according to claim 84, further comprising:
incorporating the purified oligosaccharide produced by the process into a food or feed preparation, dietary supplement, cosmetic, or pharmaceutical.

142. A method of preparing a food or feed preparation, dietary supplement, cosmetic ingredient, or pharmaceutical ingredient, the method comprising:
utilizing the purified oligosaccharide solution of claim 129 in the preparation.

143. The process of claim 84, wherein the oligosaccharide solution comprises a mammalian milk oligosaccharide (MMO).

144. The process of claim 84, wherein the oligosaccharide solution comprises a neutral human milk oligosaccharide (HMO).

145. The process of claim 84, wherein step ii) does not comprise electrodialysis.

146. The process of claim 84, wherein the purified oligosaccharide solution has an ash content below 10% (on total dry solid) with Lead content lower than 0.1 mg/kg dry solid, Arsenic content lower than 0.2 mg/kg dry solid, Cadmium content lower than 0.1 mg/kg dry solid and/or Mercury content lower than 0.5 mg/kg dry solid.
